(19) United States
(12) Patent Application Publication
(10) Pub. No.: US 2018/0116983 A1
(43) Pub. Date: May 3, 2018

(54) COMPOUNDS FOR TREATING DISORDERS ASSOCIATED WITH BK CHANNEL MODULATION

(71) Applicant: Canbex Therapeutics Limited, London (GB)

(72) Inventors: David Selwood, Hertfordshire (GB); David Baker, London (GB)

(73) Assignee: Canbex Therapeutics Limited, London (GB)

(21) Appl. No.: 15/550,621

(22) PCT Filed: Feb. 12, 2016
(86) PCT No.: PCT/GB2016/050352

§ 371 Date: Aug. 11, 2017

(30) Foreign Application Priority Data
Feb. 13, 2015 (GB) 1502415.1
Sep. 17, 2015 (GB) 1516494.0

(51) Int. Cl. A61K 31/166; A61K 31/277; A61K 31/426; A61K 31/40; A61K 31/4453; A61K 31/4412; A61P 27/06; A61P 3/10; A61P 9/10; A61P 25/18

(52) U.S. Cl. CPC A61K 31/166; A61K 31/277; A61K 31/426; A61K 31/40; A61P 25/18; A61K 31/4412; A61P 27/06; A61P 3/10; A61P 9/10; A61K 31/4453

(57) ABSTRACT

The present invention relates to a compound of formula I, or a pharmaceutically acceptable salt, solvate or prodrug thereof, wherein: Z is OR¹⁶ or NR¹⁷R¹⁸; R¹⁶ is H or alkyl; R¹⁷ is H or alkyl; R¹⁸ is alkyl or cycloalkyl, each of which is optionally substituted by one or more substituents selected from OH, halogen and COOR¹¹; X is a group selected from —C≡C—<CH₂)ₚ—; —C<R⁵)═C(R⁶)—(CH₂)_{q}—; and —C(R⁵)(R⁶)C(R⁷)(R⁸)—(CH₂)₂—; where each of R5, R6, R7 and R8 is independently II or alkyl, and each of p, q and r is independently 1, 2, 3, 4 or 5; Y is a group selected from: CN; COOR²; CONR³R⁴; SO₂NR⁹R¹⁰; NR¹²COR¹³; NR¹⁴SO₂R¹⁵; and a heterocyclic group selected from oxadiazolyl, thiazolyl, iso- thiazolyl, oxazolyl, iso-oxazolyl, pyrazoiyl and it-nidazolyl; where each of R², R³ and R⁴ is independently H or alkyl; or R³ and R⁴ are linked, together with the nitrogen to which they are attached, to form a 5 or 6-membered heterocycloalkyl or heterocycloalkenyl group, said heterocycloalkyl or heterocycloalkenyl group optionally containing one or more further groups selected from O, N, CO and S, and where each of R⁹, R¹⁰, R¹¹, R¹², R¹³, R¹⁴ and R¹⁵ is independently H or alkyl; for use in treating in treating a disorder associated with BK channel modulation.

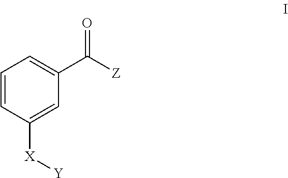

The present invention relates to compounds useful in the treatment of disorders associated with BK channel modulation.

### BACKGROUND TO THE INVENTION

VSN-16 and related compounds were first disclosed in WO 2005/080316 (University College London).

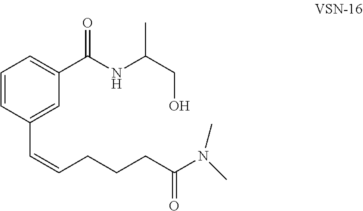 [SMILES: CC(CO)NC(=O)c1cccc(/C=C\CCCC(=O)N(C)C)c1]

VSN16 and its analogues were reported to have activity against multiple sclerosis, muscle spasticity and related muscular disorders (Hoi et at; Br J Pharmacal, 2007 November; 152(5):751-64). Hoi et at reported that VSN-16 relaxed mesenteric arteries in an endothelium-dependent manner. The vasorelaxation was antagonized by high concentrations of the classical cannabinoid antagonists, rimonabant and AM 251, as well as by O-1918, an antagonist at the abnormal-cannabidiol receptor but not at CB1 or CB2 receptors. Based on these results, the authors concluded that an additional cannabinoid receptor (or receptors) different from either the CB1 or the CB2 receptor was most likely responsible for the actions of VSN16.

Subsequent studies by the present applicant have revealed that VSN16R and analogues thereof directly activate K⁺-channels, more specifically, the large Ca²⁺ activated K⁺ channel BK channel, a known regulator of hyper-excitability.

The present invention therefore seeks to provide new therapeutic applications for VSN16 and related analogues based on this additional knowledge on its mechanism of action.

### Statement of Invention

A first aspect of the invention relates to a compound of formula I, or a pharmaceutically acceptable salt, solvate or prodrug thereof,

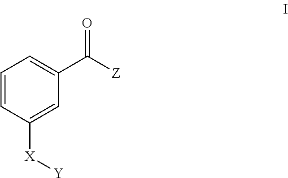

wherein:

Z is OR¹⁶ or NR¹⁷R¹⁸;

R¹⁶ is H or alkyl;

R¹⁷ is H or alkyl;

R¹⁸ is alkyl, aralkyl or cycloalkyl, each of which is optionally substituted by one or more substituents selected from OH, halogen and COOR¹¹;

X is a group selected from
—C≡C—(CH₂)ₚ—;
—C(R⁵)═C(R⁶)—(CH₂)_{q}—; and
—C(R⁵)(R⁶)C(R⁷)(R⁸)—(CH₂)ᵣ—;

where each of R⁵, R⁶, R⁷ and R⁸ is independently H or alkyl, and each of p, q and r is independently 1, 2, 3, 4 or 5;

Y is a group selected from:
CN;
COOR²;
CONR³R⁴;
SO₂NR⁹R¹⁰;
NR¹²COR¹³;
NR¹⁴SO₂R¹⁵; and
a heterocyclic group selected from oxadiazolyl, thiazolyl, iso-thiazolyl, oxazolyl, iso-oxazolyl, pyrazolyl and imidazolyl;

where each of R², R³ and R⁴ is independently H or alkyl; or R³ and R⁴ are linked, together with the nitrogen to which they are attached, to form a 5 or 6-membered heterocycloalkyl or heterocycloalkenyl group, said heterocycloalkyl or heterocycloalkenyl group optionally containing one or more further groups selected from O, N, CO and S, and where each of R⁹, R¹⁰, R¹¹, R¹², R¹³, R¹⁴ and R¹⁵ is independently H or alkyl;

for use in treating in treating a disorder associated with BK channel modulation.

### DETAILED DESCRIPTION

The present applicant has discovered that VSN16R and analogues thereof directly activate K⁺-channels, more specifically, the large Ca²⁺ activated K⁺ channel BK channel, a known regulator of hyper-excitability. Whilst the activity of VSN16R was originally believed to be “cannabinoid-like” (Hol, et al 2007), studies by the present applicant have demonstrated that the relaxing effect of VSN16R on the mesenteric artery can be inhibited by the specific BK channel blocker iberiotoxin. Furthermore, patch clamp analysis suggests that VSN16R's mediated effect on the BK channel is direct, and that blockade can be achieved by a chemically distinct BK blocker, paxilline.

In the light of this knowledge, VSN16R and related compounds have therapeutic applications in a number of indications in which activation of BK channels is reported. These include, for example, glaucoma, tinnitus, Fragile X, arterial hypertension, stroke, ischemic heart disease, psychosis, vascular dysfunction, erectile dysfunction. The compounds also have applications in providing neuroprotection, and in cardioplegia or cardiopulmonary bypass.

BK channels (BKCa channels, Maxi-K channels, large-conductance Ca²⁺-activated K⁺ channels, KCa1.1, KCNMA1, SloI) are expressed in a wide variety of cells including most neurons, muscle, epithelia, and endocrine cells. The pore-forming α-subunit of the BK channels is coded for by the single gene KCNM1, but the diversity of the BK channels is largely due to a number of C-terminal splice variants. The diversity is further increased by the presence of several accessory β-subunits, which modulate the function of the channels and are coded for by the KCNMB1-4 genes (Salkoff L. et al Nat Rev, 2006, 7(12), 921-931; Nourian, Z., M. Li, M. D. Leo, J. H. Jaggar, A. P. Braun and M. A. Hill (2014), “Large conductance Ca²⁺⁻activated K⁺ channel (BKCa) alpha-subunit splice variants in resistance arteries from rat cerebral and skeletal muscle vasculature,” PLoS One 9(6): e98863).

The BK channel complex is composed of 4 α-subunits, each spanning the membrane 7 times, plus 1-4 β-subunits (β1-β4), each spanning the membrane twice with their C and N termini internally. The α-subunits have voltage-sensors in the fourth transmembrane segment and have a classical K⁺ selectivity filter. The reason for the high conductance is two rings each with 8 negative charges located at the inner and outer mouth of the pore as well as a large negatively charged outer pore vestibule accumulating the K⁺ ions (Carvacho, I. et al, Gen Physiol, 2008,131(2), 147-161).

BK channels are unique amongst ion channels in that they are activated by depolarizing membrane potentials as well as by an increase in the intracellular Ca²⁺ concentration binding to a C-terminal site, i.e. they are voltage sensitive and calcium sensitive. This dual regulation allows BK to couple intracellular signalling to membrane potential and significantly modulate physiological responses, such as neuronal signalling and muscle contraction. In addition to this composite regulation pattern, the activity of BK channels can be further modulated by phosphorylation (protein kinases, A, C, G and CaMKII), pH, endogenous messengers (NO, cAMP, cGMP) and drugs. Since the BK channel activity is modulated by these pathways and especially by the intracellular Ca²⁺ concentration as well as by the presence of the β1 subunit, drugs interacting with these mechanisms will indirectly change the BK channel activity.

Many different chemical entities have been found to increase the activity of BK channels. Within these entities, differences in calcium dependency, subunit composition and drug binding sites have been found. Based on their origin and structure the chemical entities can be classified in: (A) endogenous BK channel modulators and structural analogs; (B) naturally occurring BK channel openers and structural analogues; (C) synthetic BK channel openers (see Nardi and Olesen, Current Medicinal Chemistry 2008, 15, 1126-1146).

As used herein “BKCa channel activation” or “BKCa activation” refers to an increase in activity at the BKCa channel relative to baseline activity (i.e. activity in the absence of said moiety). Suitable methods for determining the activity of channels such as the BKCa channel will be familiar to a person skilled in the art. For example, the ability of a particular compound to act as a BKCa channel activator can be determined by a patch clamp experiment (see Examples section for further details). For a purported BKCa channel activator, a statistically significant increase in the number of single channel openings (spikes in the patch clamp trace) is indicative of BKCa channel activity.

Compounds

The present invention relates to compounds of formula I as defined herein, and pharmaceutically acceptable salts, solvates and prodrugs thereof, for use in treating in treating a disorder associated with BK channel modulation.

As used herein, the term “alkyl” includes both saturated straight chain and branched alkyl groups which may be substituted (mono- or poly-) or unsubstituted. Preferably, the alkyl group is a C₁₋₂₀ alkyl group, more preferably a C₁₋₁₅, more preferably still a C₁₋₁₀ alkyl group, more preferably still, a C₁₋₆ alkyl group or a C₁₋₄ alkyl group. Particularly preferred alkyl groups include, for example, methyl, ethyl, propyl, isopropyl, butyl, isobutyl, tert-butyl, pentyl and hexyl. Suitable substituents include, for example, alkyl, hydroxy, halo-, alkoxy-, nitro-, COOH, CO₂-alkyl, alkenyl, CN, NH₂ and CF₃.

As used herein, the term “cycloalkyl” refers to a cyclic alkyl group which may be substituted (mono- or poly-) or unsubstituted. Preferably, the cycloalkyl group is a C₃₋₆-cycloalkyl group. Suitable substituents include, for example, alkyl, hydroxy, halo-, alkoxy-, nitro-, COOH, CO₂-alkyl, alkenyl, CN, NH₂ and CF₃.

As used herein, the term “alkenyl” refers to group containing one or more double bonds, which may be branched or unbranched, and substituted (mono- or poly-) or unsubstituted. Preferably the alkenyl group is a C₂₋₂₀ alkenyl group, more preferably a C₂₋₁₅ alkenyl group, more preferably still a C₂₋₁₀ alkenyl group, or preferably a C₂₋₈ alkenyl group. Suitable substituents include, for example, alkyl, hydroxy, halo-, alkoxy-, nitro-, COOH, CO₂-alkyl, alkenyl, CN, NH₂ and CF₃.

As used herein, the term “aryl” refers to a C₆₋₁₀ aromatic group which may be substituted (mono- or poly-) or unsubstituted. Typical examples include phenyl and naphthyl etc. Suitable substituents include, for example, alkyl, hydroxy, halo-, alkoxy-, nitro-, COOH, CO₂-alkyl, alkenyl, CN, NH₂ and CF₃.

As used herein, the term “aralkyl” includes, but is not limited to, a group having both aryl and alkyl functionalities. By way of example, the term includes groups in which one of the hydrogen atoms of the alkyl group is replaced by an aryl group, e.g. a phenyl group optionally having one or more substituents such as halo, alkyl, alkoxy, hydroxy, and the like. Typical aralkyl groups include benzyl, phenethyl and the like.

As used herein, the term “heterocycle” (also referred to herein as “heterocyclyl” and “heterocyclic”) refers to a substituted (mono- or poly-) or unsubstituted saturated, unsaturated or partially unsaturated cyclic group containing one or more heteroatoms selected from N, O and S, and which optionally further contains one or more CO groups. Suitable substituents include, for example, halo, alkyl, alkoxy, hydroxy, and the like. The term “heterocycle” encompasses both heteroaryl groups and heterocycloalkyl groups as defined below.

As used herein, the term “heteroaryl” refers to a C₂₋₁₂ aromatic, substituted (mono- or poly-) or unsubstituted group, which comprises one or more heteroatoms. Preferably, the heteroaryl group is a C₄₋₁₂ aromatic group comprising one or more heteroatoms selected from N, O and S. Suitable heteroaryl groups include pyrrole, pyrazole, pyrimidine, pyrazine, pyridine, quinoline, thiophene, 1,2,3-triazole, 1,2,4-triazole, thiazole, oxazole, iso-thiazole, iso-oxazole, imidazole, furan and the like. Suitable substituents include, for example, alkyl, hydroxy, halo-, alkoxy-, nitro-, COOH, CO₂-alkyl, alkenyl, CN, NH₂, CF₃ and cyclic groups.

As used herein, the term “heterocycloalkyl” refers to a substituted (mono- or poly-) or unsubstituted cyclic aliphatic group which contains one or more heteroatoms. Preferred heterocycloalkyl groups include piperidinyl, pyrrolidinyl, piperazinyl, thiomorpholinyl and morpholinyl. More preferably, the heterocycloalkyl group is selected from N-piperidinyl, N-pyrrolidinyl, N-piperazinyl, N-thiomorpholinyl and N-morpholinyl.

As used herein, the term “heterocycloalkenyl” refers to a substituted (mono- or poly-) or unsubstituted cyclic group which contains one or more heteroatoms and one or more carbon-carbon double bonds.

In one preferred embodiment, R¹⁸ is alkyl or cycloalkyl, each of which is optionally substituted by one or more substituents selected from OH, halogen and COOR¹¹.

In one preferred embodiment, R¹⁷ is H and R¹⁸ is selected from alkyl and cycloalkyl, each of which is optionally substituted by one or more substituents selected from OH and F.

In one preferred embodiment, Z is OR¹⁸ and R¹⁸ is alkyl.

In one preferred embodiment, Z is NR¹⁷R¹⁸.

In one preferred embodiment, the invention relates to a compound of formula IA, or a pharmaceutically acceptable salt, solvate or prodrug thereof,

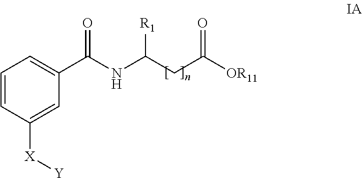

wherein:

n is 0 or 1;

R¹ is selected from H, alkyl and aralkyl, wherein said alkyl and aralkyl groups may be optionally substituted by one or more OH groups;

X is a group selected from
—C≡C—(CH₂)ₚ—;
—C(R⁵)═C(R⁶)—(CH₂)_{q}—; and
—C(R⁵)(R⁶)C(R⁷)(R⁸)—(CH₂)ᵣ—;

where each of R⁵, R⁶, R⁷ and R⁸ is independently H or alkyl, and each of p, q and r is independently 1, 2, 3, 4 or 5;

Y is a group selected from:
CN;
COOR²;
CONR³R⁴;
SO₂NR⁹R¹⁰;
NR¹²COR¹³;
NR¹⁴SO₂R¹⁵; and
a heterocyclic group selected from oxadiazolyl, thiazolyl, iso-thiazolyl, oxazolyl, iso-oxazolyl, pyrazolyl and imidazolyl;

where each of R², R³ and R⁴ is independently H or alkyl; or R³ and R⁴ are linked, together with the nitrogen to which they are attached, to form a 5 or 6-membered heterocycloalkyl or heterocycloalkenyl group, said heterocycloalkyl or heterocycloalkenyl group optionally containing one or more further groups selected from O, N, CO and S, and where each of R⁹, R¹⁰, R₁₁, R¹², R¹³, R¹⁴ and R¹⁵ is independently H or alkyl;

for use in the treatment of a disorder associated with BK channel modulation.

In one preferred embodiment, the compound for use according to the invention is a compound of formula IA, or a pharmaceutically acceptable salt or prodrug thereof,

wherein:

n is 0 or 1;

R¹ is selected from H, alkyl and aralkyl, wherein said alkyl and aralkyl groups may be optionally substituted by one or more OH groups;

X is a group selected from
—C≡C—(CH₂)ₚ—;
—C(R⁵)═C(R⁸)—(CH₂)_{q}—; and
—C(R⁵)(R⁶)C(R⁷)(R⁸)—(CH₂)ᵣ—;

where each of R⁵, R⁶, R⁷ and R⁸ is independently H or alkyl, and each of p, q and r is independently 2, 3, or 4;

Y is a group selected from:
CN;
COOR²;
CONR³R⁴;
SO₂NR⁹R¹⁰; and
a heterocyclic group selected from oxadiazolyl, thiazolyl, iso-thiazolyl, oxazolyl, iso-oxazolyl, pyrazolyl and imidazolyl;

where each of R², R³ and R⁴ is independently H or alkyl; or R³ and R⁴ are linked, together with the nitrogen to which they are attached, to form a 5 or 6-membered heterocycloalkyl group, said heterocycloalkyl group optionally containing one or more further heteroatoms selected from O, N and S, and where each of R⁹ and R¹⁰ is independently H or alkyl.

In one preferred embodiment, R¹ is selected from H, Me, Et, ⁿPr, CH₂-phenyl, CH₂-[4-(OH)-phenyl], CH₂OH, CH(OH)CH₃, CH(CH₃)CH₂CH₃ and CH₂CH(CH₃)₂. More preferably, R¹ is H, CH₂OH, Me, Et or CH₂-phenyl.

In one preferred embodiment, Y is selected from CN, CON(Me)₂, CONHMe, CONHEt, SO₂N(Me)₂, N(Me)COMe, N(Me)SO₂Me, CO-piperidinyl, CO-pyrrolidinyl, oxadiazolyl and thiazolyl. Preferably, Y is thiazol-4-yl.

In one highly preferred embodiment, Y is CON(Me)₂.

In one preferred embodiment, each of p, q and r is independently 2, 3, or 4.

In one preferred embodiment, X is —C≡C—(CH₂)ₚ—, where p is 1, 2, 3, 4, or 5.

In one preferred embodiment, X— is cis —C(R⁵)═C(R⁶)—(CH₂)_{q}— and q is 2, 3 or 4.

In one preferred embodiment, X is —CH═CH—(CH₂)_{q}— and q is 2 or 3.

In one preferred embodiment, X is —C(R⁵)(R⁶)C(R⁷)(R⁸)—(CH₂)ᵣ— and r is 2, 3 or 4.

In one preferred embodiment, X is —CH₂—CH₂—(CH₂)ᵣ— and r is 2 or 3.

In one preferred embodiment, R₁₁ is H.

In another preferred embodiment, R₁₁ is C₁₋₆-alkyl, more preferably, Me or Et, even more preferably, Me.

In one preferred embodiment, the compound for use according to the invention is of formula Ia, or a pharmaceutically acceptable salt thereof,

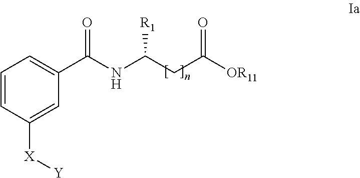

wherein R¹, R¹¹, X, Y and n are as defined above. In one preferred embodiment, R₁₁ is H.

In one preferred embodiment, the compound for use according to the invention is of formula Ib, or a pharmaceutically acceptable salt thereof,

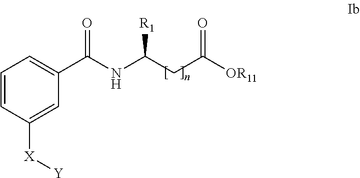

wherein R¹, R¹¹, X, Y and n are as defined above. In one preferred embodiment, R₁₁ is H.

In one preferred embodiment, n is 0.

In one preferred embodiment, n is 1.

In one preferred embodiment, R¹ is Me.

In one preferred embodiment, R¹ is CH₂OH.

In one preferred embodiment, R¹ is CH₂Ph.

In one preferred embodiment, R¹ is H.

In one preferred embodiment, n is 0, R¹ is Me and X is —CH═CH—(CH₂)₃— or —CH₂—CH₂—(CH₂)₃—.

In one preferred embodiment, n is 1 or 2 and R¹ is H.

In one preferred embodiment, n is 1 and R¹ is H.

In one preferred embodiment of the invention, the compound for use according to the invention is selected from the following:

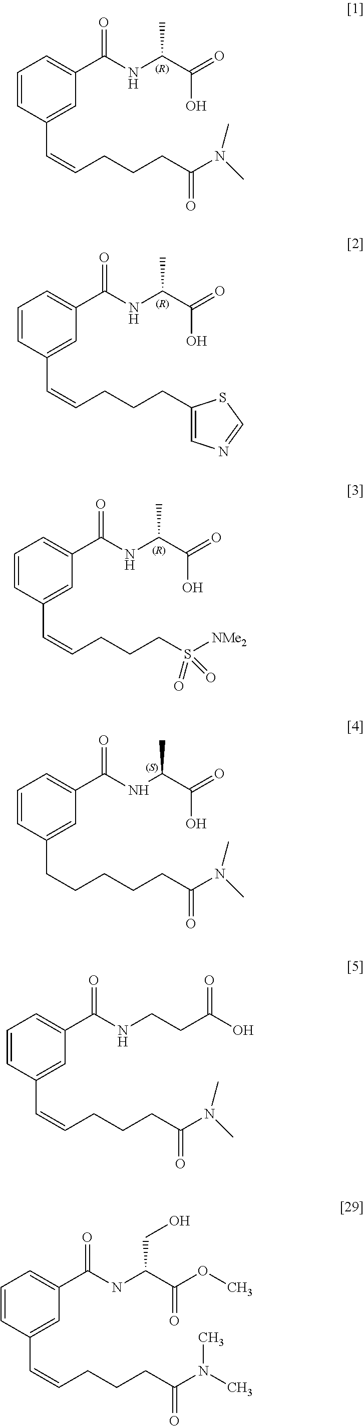

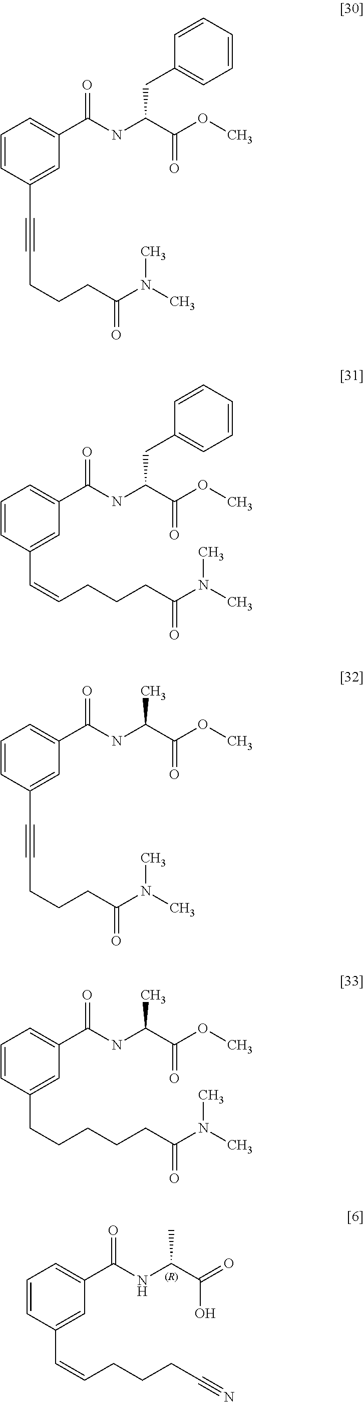

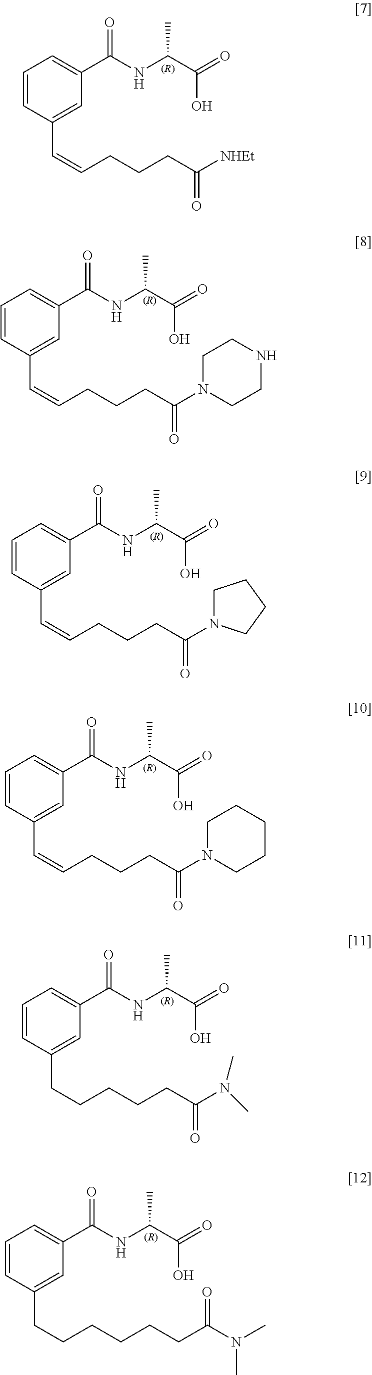

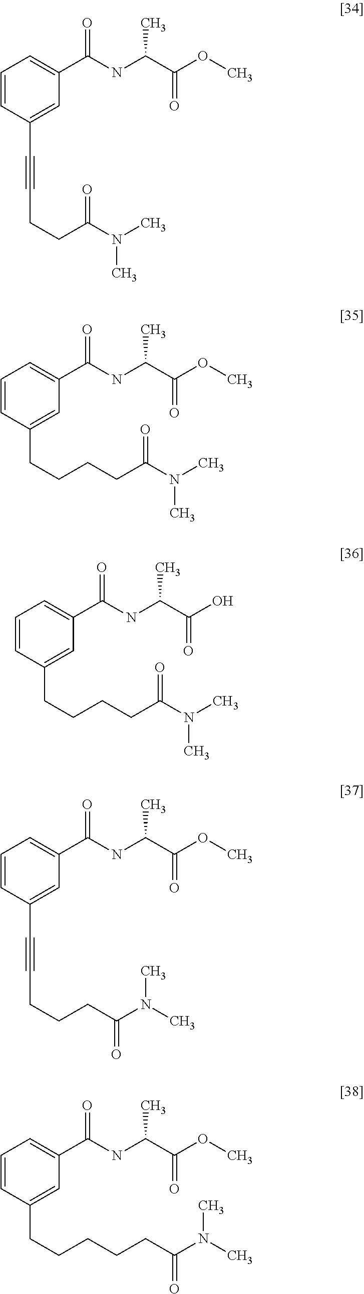 [SMILES: COC(=O)[C@@H](C)NC(=O)c1cccc(C#CCCC(=O)N(C)C)c1 ; COC(=O)[C@@H](C)NC(=O)c1cccc(CCCCC(=O)N(C)C)c1 ; C[C@@H](NC(=O)c1cccc(CCCCC(=O)N(C)C)c1)C(=O)O ; COC(=O)[C@@H](C)NC(=O)c1cccc(C#CCCCC(=O)N(C)C)c1 ; COC(=O)[C@@H](C)NC(=O)c1cccc(CCCCCC(=O)N(C)C)c1]

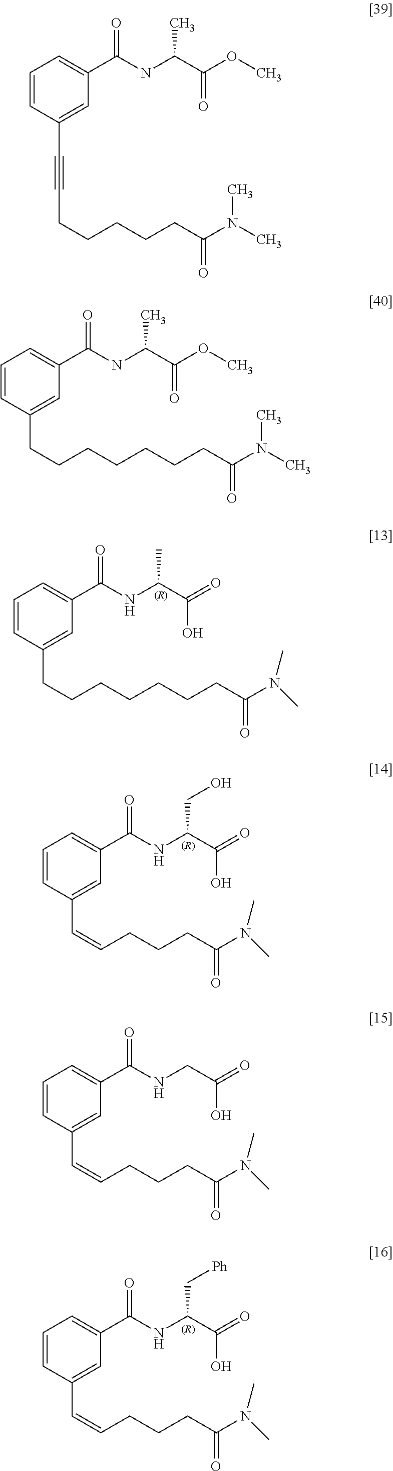

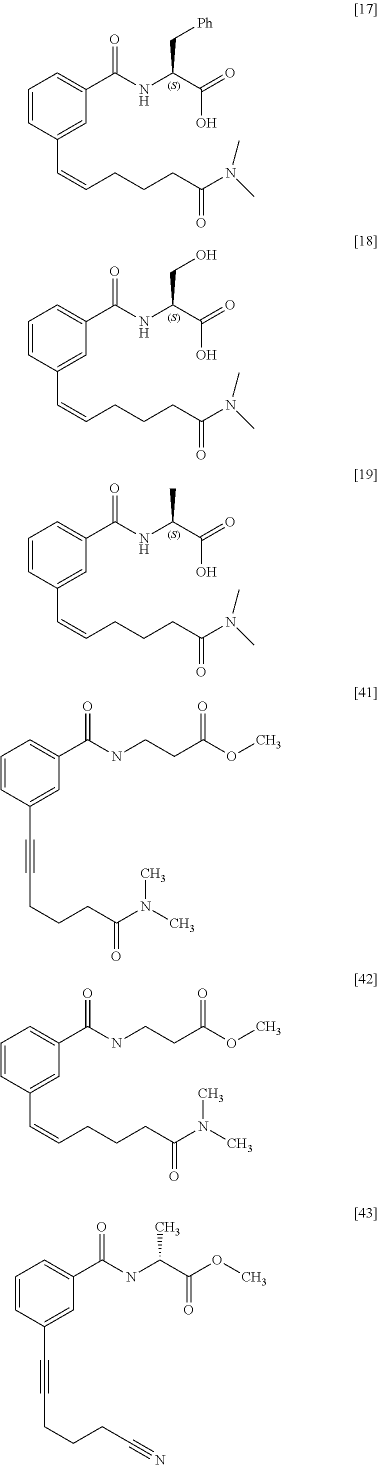

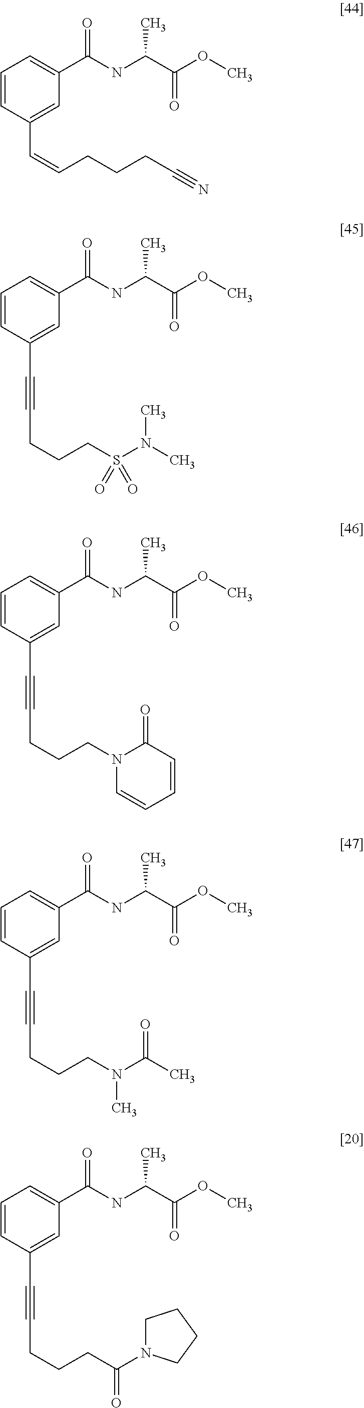 [SMILES: COC(=O)[C@@H](C)NC(=O)c1cccc(/C=C\CCCC#N)c1 ; COC(=O)[C@@H](C)NC(=O)c1cccc(C#CCCCS(=O)(=O)N(C)C)c1 ; COC(=O)[C@@H](C)NC(=O)c1cccc(C#CCCCn2ccccc2=O)c1 ; COC(=O)[C@@H](C)NC(=O)c1cccc(C#CCCCN(C)C(C)=O)c1 ; COC(=O)[C@@H](C)NC(=O)c1cccc(C#CCCCC(=O)N2CCCC2)c1]

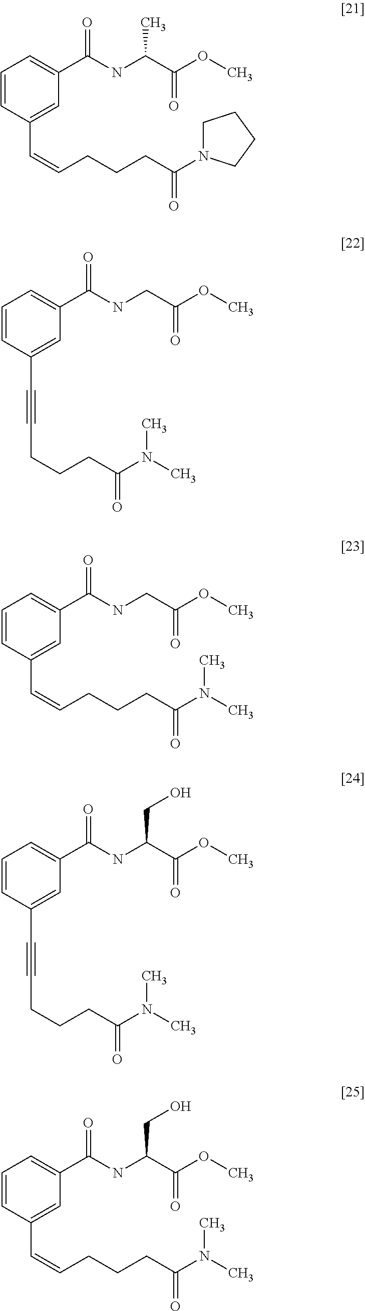 [SMILES: COC(=O)[C@@H](C)NC(=O)c1cccc(/C=C\CCCC(=O)N2CCCC2)c1 ; COC(=O)CNC(=O)c1cccc(C#CCCCC(=O)N(C)C)c1 ; COC(=O)CNC(=O)c1cccc(/C=C\CCCC(=O)N(C)C)c1 ; COC(=O)[C@H](CO)NC(=O)c1cccc(C#CCCCC(=O)N(C)C)c1 ; COC(=O)[C@H](CO)NC(=O)c1cccc(/C=C\CCCC(=O)N(C)C)c1]

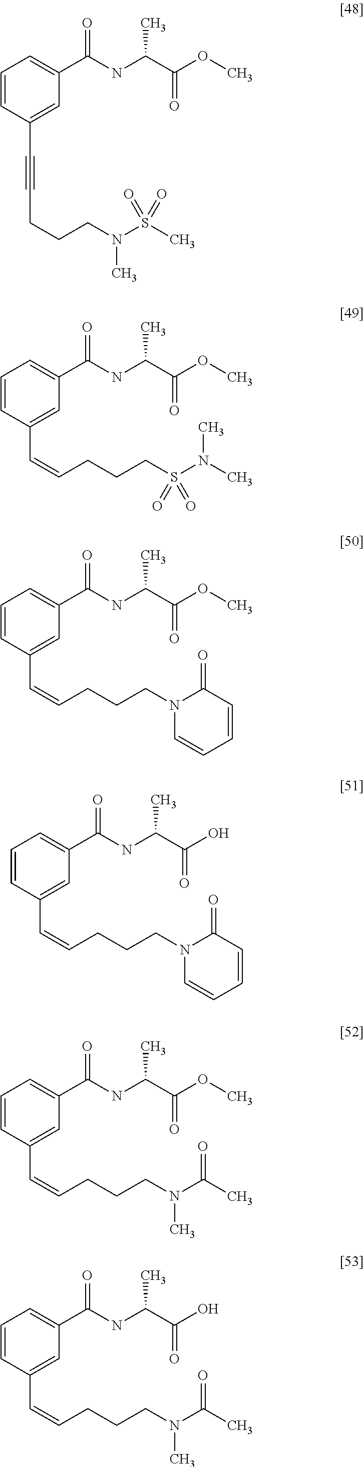 [SMILES: COC(=O)[C@@H](C)NC(=O)c1cccc(C#CCCCN(C)S(C)(=O)=O)c1 ; COC(=O)[C@@H](C)NC(=O)c1cccc(/C=C\CCCS(=O)(=O)N(C)C)c1 ; COC(=O)[C@@H](C)NC(=O)c1cccc(/C=C\CCCn2ccccc2=O)c1 ; C[C@@H](NC(=O)c1cccc(/C=C\CCCn2ccccc2=O)c1)C(=O)O ; COC(=O)[C@@H](C)NC(=O)c1cccc(/C=C\CCCN(C)C(C)=O)c1 ; CC(=O)N(C)CCC/C=C\c1cccc(C(=O)N[C@H](C)C(=O)O)c1]

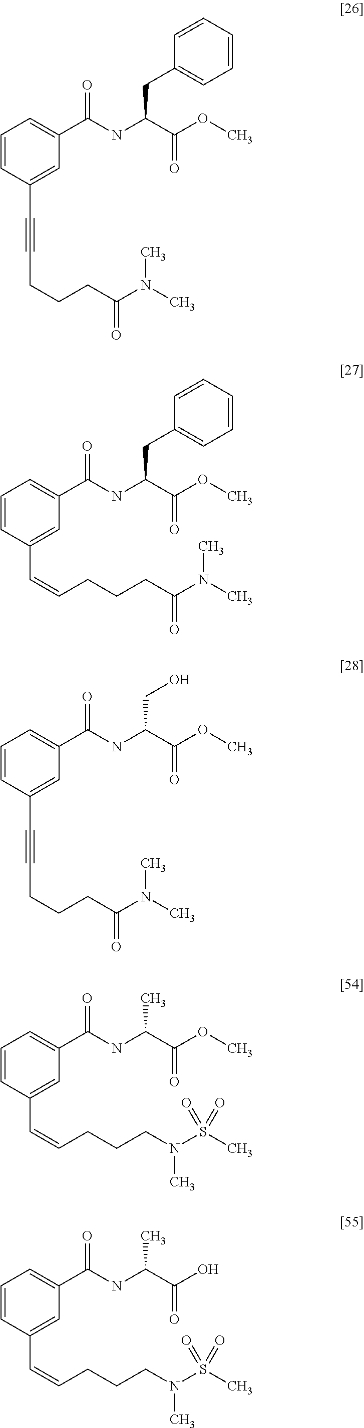 [SMILES: COC(=O)[C@H](Cc1ccccc1)NC(=O)c1cccc(C#CCCCC(=O)N(C)C)c1 ; COC(=O)[C@H](Cc1ccccc1)NC(=O)c1cccc(/C=C\CCCC(=O)N(C)C)c1 ; COC(=O)[C@@H](CO)NC(=O)c1cccc(C#CCCCC(=O)N(C)C)c1 ; COC(=O)[C@@H](C)NC(=O)c1cccc(/C=C\CCCN(C)S(C)(=O)=O)c1 ; C[C@@H](NC(=O)c1cccc(/C=C\CCCN(C)S(C)(=O)=O)c1)C(=O)O]

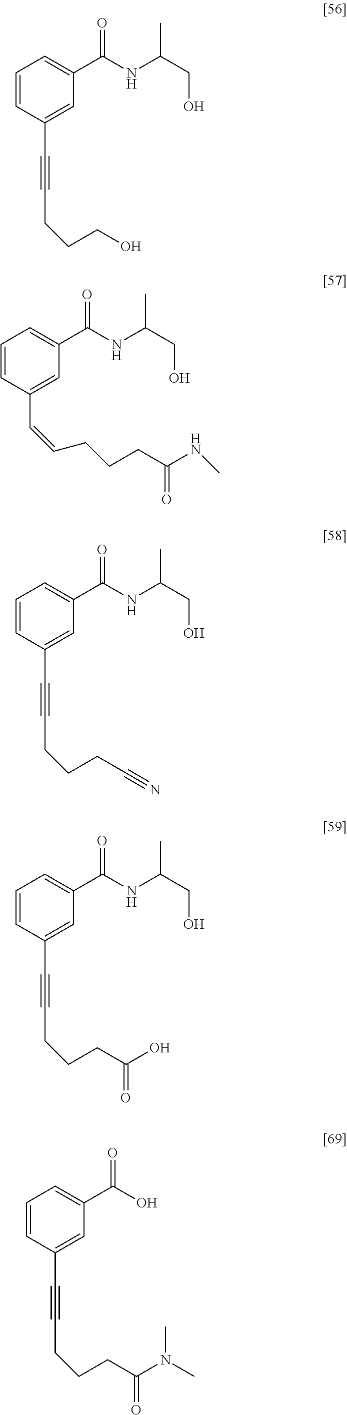 [SMILES: CC(CO)NC(=O)c1cccc(C#CCCCO)c1 ; CNC(=O)CCC/C=C\c1cccc(C(=O)NC(C)CO)c1 ; CC(CO)NC(=O)c1cccc(C#CCCCC#N)c1 ; CC(CO)NC(=O)c1cccc(C#CCCCC(=O)O)c1 ; CN(C)C(=O)CCCC#Cc1cccc(C(=O)O)c1]

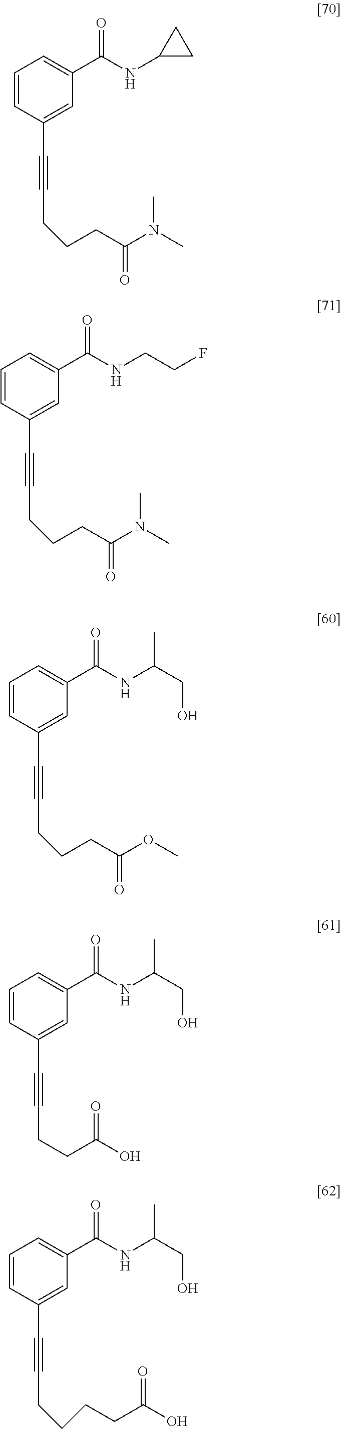 [SMILES: CN(C)C(=O)CCCC#Cc1cccc(C(=O)NC2CC2)c1 ; CN(C)C(=O)CCCC#Cc1cccc(C(=O)NCCF)c1 ; COC(=O)CCCC#Cc1cccc(C(=O)NC(C)CO)c1 ; CC(CO)NC(=O)c1cccc(C#CCCC(=O)O)c1 ; CC(CO)NC(=O)c1cccc(C#CCCCCC(=O)O)c1]

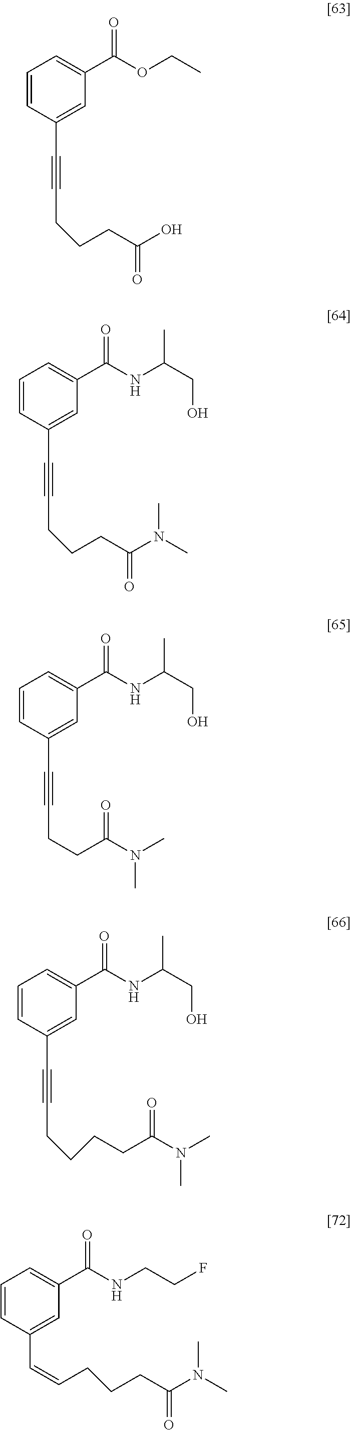 [SMILES: CCOC(=O)c1cccc(C#CCCCC(=O)O)c1 ; CC(CO)NC(=O)c1cccc(C#CCCCC(=O)N(C)C)c1 ; CC(CO)NC(=O)c1cccc(C#CCCC(=O)N(C)C)c1 ; CC(CO)NC(=O)c1cccc(C#CCCCCC(=O)N(C)C)c1 ; CN(C)C(=O)CCC/C=C\c1cccc(C(=O)NCCF)c1]

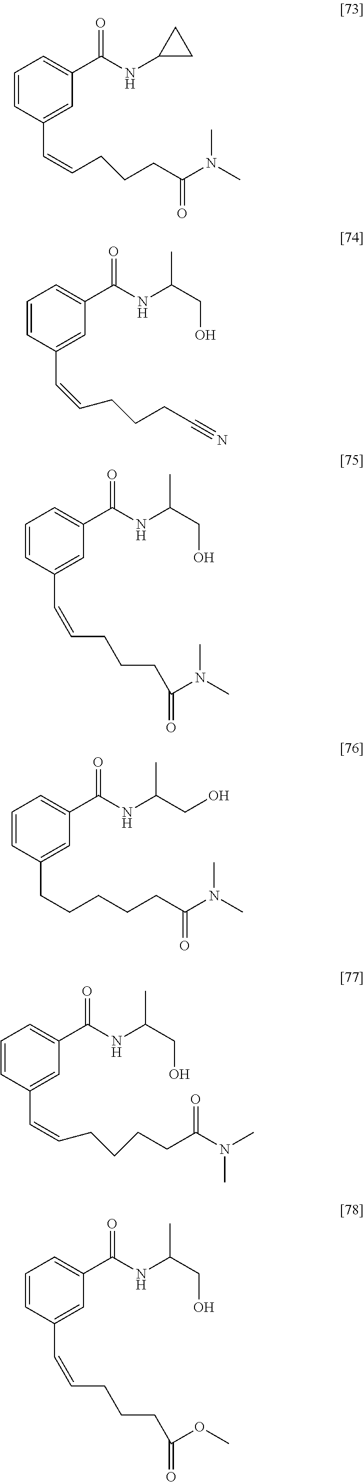

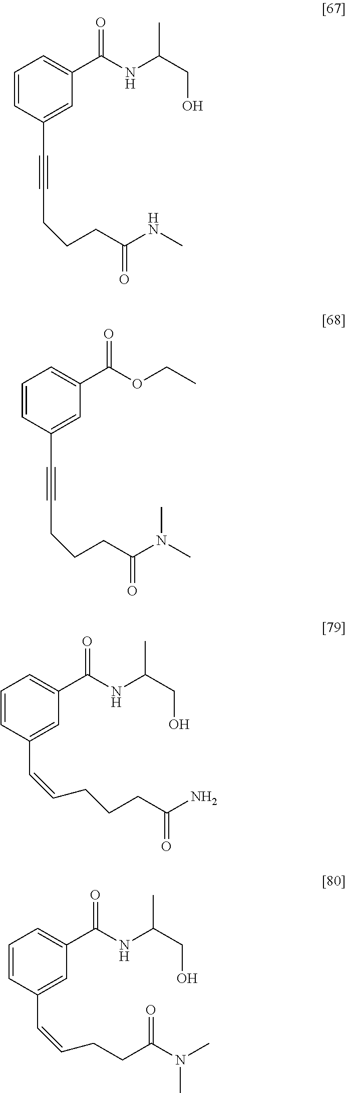 [SMILES: CNC(=O)CCCC#Cc1cccc(C(=O)NC(C)CO)c1 ; CCOC(=O)c1cccc(C#CCCCC(=O)N(C)C)c1 ; CC(CO)NC(=O)c1cccc(/C=C\CCCC(N)=O)c1 ; CC(CO)NC(=O)c1cccc(/C=C\CCC(=O)N(C)C)c1]

and pharmaceutically acceptable salts, solvates, prodrugs and enantiomers thereof, and mixtures of said enantiomers. In the table above, and in the structures shown throughout the specification, for ease of presentation the hydrogen on the amide nitrogen is not always shown. However, a person skilled in organic chemistry would clearly understand the nature of the chemical structures depicted. Compounds 56-80, and methods for the preparation thereof, are described in WO 2005/080316. Compounds 1-55 and methods for the preparation thereof, are described in WO 2015/082938.

In one especially preferred embodiment, the compound for use according to the invention is of formula [1], or a pharmaceutically acceptable salt or prodrug thereof:

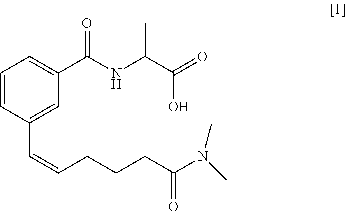 [SMILES: CC(NC(=O)c1cccc(/C=C\CCCC(=O)N(C)C)c1)C(=O)O]

More preferably still, the compound for use according to the invention is of formula [1a] or formula [1b], or a mixture thereof:

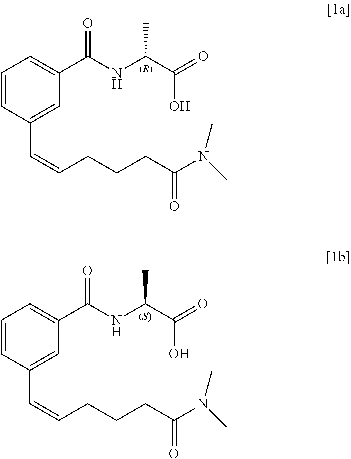

In one preferred embodiment, the compound for use according to the invention is a racemic mixture of compounds [1a] and [1b].

In one preferred embodiment, the compound for use according to the invention is of formula [75], or a pharmaceutically acceptable salt or prodrug thereof:

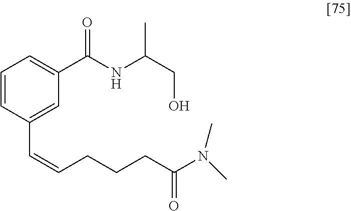 [SMILES: CC(CO)NC(=O)c1cccc(/C=C\CCCC(=O)N(C)C)c1]

More preferably still, the compound for use according to the invention is of formula [2a] or formula [75b], or a mixture thereof:

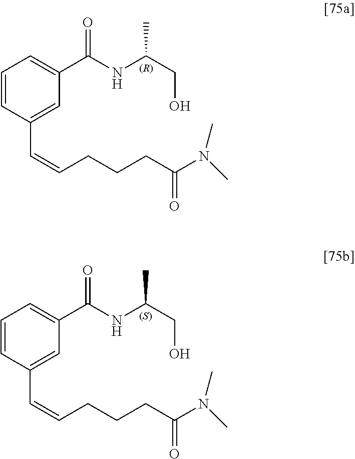

In one preferred embodiment, the compound for use according to the invention is of formula [57], or a pharmaceutically acceptable salt or prodrug thereof:

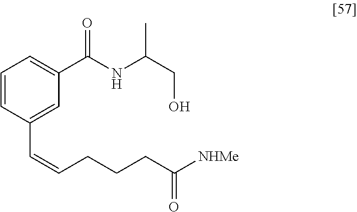 [SMILES: CNC(=O)CCC/C=C\c1cccc(C(=O)NC(C)CO)c1]

More preferably still, the compound for use according to the invention is of formula [57a] or formula [57b], or a mixture thereof:

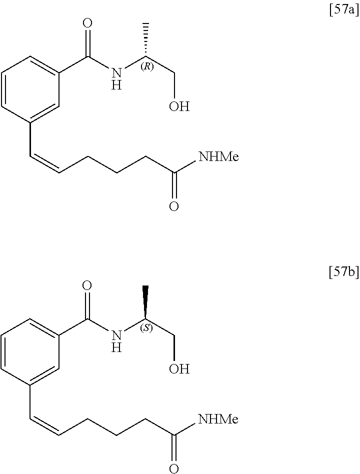

Therapeutic Applications

The compounds according to the invention are for use in treating disorders associated with BK channel modulation.

In one embodiment, the disorder is one associated with abnormal BK channel activity.

BKCa dysfunction has been observed in the Fmr1-/y mouse in vivo using voltage sensitive dyes to detect voltage changes in craniotomies. These studies show increased excitability and lateral spread of excitation (Zhang et al, Nature Neuroscience 2014, 1701-1709).

In one preferred embodiment, the disorder involves dysregulation of BK activity, for example, there is a dysfunctional level of BK channel activity in an organism resulting from disruption of the normal function of a regulatory mechanism. This may also arise in certain diseases, particularly ones where mutations in the BK channel are implicated.

In one preferred embodiment, the disorder is one associated with the impairment of BK channel activity.

Cells/neurones may be excitable for different reasons, such as prolonged depolarization due to the loss of myelin. Thus, opening the BK channel serves to repolarize or hyperpolarize the cell, raising the action potential threshold, decreasing firing and restoring normal excitability of the system.

In one preferred embodiment, the disorder is one associated with reduced BK channel activity compared to baseline activity.

As used herein the term “disorder” refers to any mental or physiological problem that interrupts normal function in a subject. Disorders include conditions, diseases, illnesses, injuries, disabilities, syndromes, infections, isolated symptoms, and atypical variations of structure and function.

Studies have shown that the compounds described herein act as BK channel openers. Further details of patch clamp studies demonstrating this effect are provided in the accompanying examples. More specifically, inside-out patch clamp studies show that the activation of BK by VSN16R is voltage and calcium dependent and occurs optimally on depolarized cells between +20 and +80 mV.

Cells/neurones may be excitable for different reasons, such as prolonged depolarization due to the loss of myelin. Thus, opening the BK channel serves to repolarize or hyperpolarize the cell, raising the action potential threshold, decreasing firing and restoring normal excitability of the system.

Molecular characterisation studies were undertaken to elucidate further details of the mechanism of VSN16R. The EA.hy926 endothelial cell line is a cell line responsive to VSN16R. More specifically, VSN16R induces a BK_{ca} specific current in whole EA.hy926 cells, which can be blocked by paxilline. It also induces sustained hyperpolarisation. In inside-out patch clamp experiments, VSN16R increases the open probability of BK_{ca} channels. Taking into consideration the functional diversity of BK_{ca} channels, which reflects their structural complexity, the applicant sought to clarify which BK_{ca} channel α isoforms and β subunits are present in the EA.hy926 cells in order to define the isoform selectivity of VSN16R.

Previously published data (Hoi et al, 2007) shows that VSN16R does not act on smooth muscle, carrying the β1 subunit. Moreover, data on the recombinant ZERO isoform alone expressed in HEK293 cells showed that these cells are not responsive to VSN16R. RNA sequencing performed on the spinal cord homogenates of EAE mice showed that the β4, and to a much lesser extent β2, isoforms are expressed along with the α.

In addition to the human EA.hy926 endothelial cells, the alpha-1/beta 4 subunit combination is expressed in the plasma membrane and mitochondria of neuronal cells (Wang B, Jaffe D B, Brenner R (2014); Current understanding of iberiotoxin-resistant BK channels in the nervous system; Frontiers in Physiology 5:382). Diseases that involve this combination of subunits will therefore be particularly well suited to treatment with the compounds of the invention, e.g. including but not limited to glaucoma, tinnitus, Fragile X, diabetic reinopathy, stroke, psychosis, vascular dysfunction, other ocular diseases such as Age Related Macular Degeneration and retinitis pigmentosa, and in neuroprotection.

Detailed mechanistic studies by the applicant have confirmed that compounds of formula I, including VSN16 and related analogues, do not activate the alpha-1 subunit, but preferably bind to the β4 subunit.

In one preferred embodiment, the compounds are for use in treating glaucoma. Glaucoma is a term describing a group of ocular disorders resulting in optic nerve damage or loss to the field of vision, in many cases caused by a clinically characterized pressure build-up in relation to the fluid of the eye (intraocular pressure-associated optic neuropathy). The disorders can be roughly divided into two main categories, “open-angle” and “closed-angle” (or “angle closure”) glaucoma. The angle refers to the area between the iris and cornea, through which fluid must flow to escape via the trabecular meshwork, an area of tissue in the eye located around the base of the cornea. Closed-angle glaucoma can appear suddenly and is often painful; visual loss can progress quickly, but the discomfort often leads patients to seek medical attention before permanent damage occurs. Open-angle, chronic glaucoma tends to progress at a slower rate and patients may not notice they have lost vision until the disease has progressed significantly.

Increased intraocular pressure can permanently damage vision in the affected eye(s) and lead to blindness if left untreated. The nerve damage involves loss of retinal ganglion cells in a characteristic pattern. When the scleral venous sinus is blocked to where aqueous humor is not reabsorbed at a faster rate than it is being secreted, elevated pressure within the eye occurs. Pressure in the anterior and posterior chambers pushes the lens back and puts pressure on the vitreous body. The vitreous body presses the retina against the choroid and compresses the blood vessels that feed the retina. Without a sufficient blood supply, retinal cells will die and the optic nerve may atrophy, causing blindness. Typically, the nerves furthest from the focal point fail first because of their distance from the central blood supply to the eye; thus, vision loss due to glaucoma tends to start at the edges with the peripheral visual field, leading to progressively worse tunnel vision.

Glaucoma has been called the “silent thief of sight” because the loss of vision often occurs gradually over a long period of time, and symptoms only occur when the disease is quite advanced. Once lost, vision cannot normally be recovered, so treatment is aimed at preventing further loss. Worldwide, glaucoma is the second-leading cause of blindness after cataracts and is the leading cause of blindness among African Americans. Glaucoma affects one in 200 people aged 50 and younger, and one in 10 over the age of 80.

Studies by Ellis et al have demonstrated that NO-induced regulation of the human trabecular meshwork cell volume and aqueous humor outflow facility involves the BK ion channel (Dismuke, W. M., C. C. Mbadugha and D. Z. Ellis, 2008; Am J Physiol Cell Physiol 294(6): C1378-1386). Moreover, recent studies by the applicant have shown that the application of topical VSN16R to the eye reduces intraocular pressure in normal rats. The compounds of the invention are also capable of acting on the plasma membrane and mitochondrial BKCa channels of retinal ganglion cells to provide neuroprotection.

The BK channel therefore represents a therapeutic target for glaucoma, and pharmacological molecules that open the BK channel provide a promising treatment for this disorder.

In one preferred embodiment, the compounds are for use in treating tinnitus. Tinnitus is a condition that can result from a wide range of underlying causes. The most common cause is noise-induced hearing loss. Other causes include neurological damage (multiple sclerosis), ear infections, oxidative stress, emotional stress, foreign objects in the ear, nasal allergies that prevent (or induce) fluid drain, wax build-up, and exposure to loud sounds. Withdrawal from benzodiazepines may also cause tinnitus. Tinnitus may be an accompaniment of sensorineural hearing loss or congenital hearing loss, or it may be observed as a side effect of certain medications (ototoxic tinnitus). The condition is often rated clinically on a simple scale from “slight” to “catastrophic” according to the difficulties it imposes, such as interference with sleep, quiet activities, and normal daily activities. Tinnitus is common, affecting about 10-15% of people. To date, there are no effective medications.

Recent studies by Lobarinas et al investigated the effects of the potassium ion channel modulator BMS-204352 (Maxipost) and its R-enantiomer on salicylate-induced tinnitus in rats (Lobarinas, E., W. Dalby-Brown, D. Stolzberg, N. R. Mirza, B. L. Allman and R. Salvi (2011), Physiol Behav 104(5): 873-879). In this study the non-selective BK/KV7 ligand BMS-204352 (Maxipost) showed that the behavioural effects of tinnitus were abolished at 10 mg/kg. R-Maxipost which loses Kv7 activity but maintains its BK activity maintained the positive effect on tinnitus indicating that Kv channels were not involved in the Maxipost effect. The BK channel therefore represents a therapeutic target for tinnitus, and pharmacological molecules that open the BK channel provide a promising treatment for this disorder.

In one preferred embodiment, the compounds of the invention are for use in treating Fragile X. Fragile X syndrome (FXS), also known as Martin-Bell syndrome, or Escalante's syndrome (more commonly used in South American countries), is a genetic syndrome that is a single-gene cause of autism and inherited cause of intellectual disability, especially among boys. FXS is characterized by intellectual disability, social anxiety, attention-deficit hyperactivity disorder and abnormal physical characteristics (Hagerman, 1997), such as an elongated face, large or protruding ears, and large testes (macroorchidism), and behavioral characteristics such as stereotypic movements (e.g. hand-flapping). FXS is identified as an urgent unmet need for effective treatment due to the rapidly growing patient population and the consequent huge burden on affected individuals, their families and caregivers, and society as a whole. There is currently no drug treatment that has shown benefit specifically for fragile X syndrome. However, medications are commonly used to treat symptoms of attention deficit and hyperactivity, anxiety, and aggression. Supportive management is important in optimizing functioning in individuals with fragile X syndrome, and may involve speech therapy, occupational therapy, and individualized educational and behavioral programs.

FXS is a monogenic neurodevelopmental disorder that can be caused by mutation due to a genetic expansion of CGG trinucleotide repeats in the Fragile X-Mental Retardation 1 (Fmr1) gene on the X chromosome. This results in a failure to express the fragile X mental retardation protein (FMRP), which is required for normal neural development. Depending on the length of the CGG repeat, an allele may be classified as normal (unaffected by the syndrome), a premutation (at risk of fragile X associated disorders), or full mutation (usually affected by the syndrome). A definitive diagnosis of fragile X syndrome is made through genetic testing to determine the number of CGG repeats. Testing for premutation carriers can also be carried out to allow for genetic counseling. The first complete DNA sequence of the repeat expansion in someone with the full mutation was generated by scientists in 2012 using SMRT sequencing.

In the FXS mutation the FMRI gene can trigger partial or complete gene silencing and partial or complete lack of the fragile X mental retardation protein (FMRP) (Oostra and Willemsen, 2003). In the brain, FMRP is highly expressed in neurons and is actively transported as part of a messenger RNA-protein-complex through the dendrite to the synaptic spines, where its main function appears to be the regulation of protein synthesis (Darnell & Klann, 2013). Insufficient expression of FMRP leads to deregulated translation and has a broad array of effects on cellular signaling pathways and on synaptic plasticity, morphology and function, thereby leading to abnormalities in brain connectivity and neurodevelopmental processes (Grossman et al., 2006; Bassell & Warren, 2008; Darnell et al., 2011; Bhakar et al., 2012).

It has been reported that FMR1P deletion is associated with a reduction of KCNMA1 expression (Briault & Perche 2012) and it has been found that mutations in the KCNMA1 gene was detected in someone with autistic behaviours (Laumonnier et al. 2006). Likewise a reduction of Kcnma1 expression has been found in Fmr1 knockout mice (Briault & Perche 2012). Mutant Fmr1 knockout mice recapitulate this phenotype and represent a preclinical model for assessment of putative drug treatments (Mientjes et al. 2006, Deacon et al. 2015) and it was found that opening of KCNMA1 channels with BMS-204352 (Maxipost) could inhibit the FXS-associated phenotypes of the Fmr1 knockout mouse (Hébert et al. 2014). The target of BMS-204352 is thought to act via a direct action on the channels and cytoplasmic domains (Gressner et al. 2012), which is distinct from the action of VSN16R, as VSN16R action results in membrane polarisation due to maintaining opening of the KCNMA1 pore within the BK_{ca} channel complex.

The BK channel therefore represents a therapeutic target for fragile X syndrome, and pharmacological molecules that open the BK channel provide a promising treatment for this disorder.

In one preferred embodiment, the compounds are for use in treating arterial hypertension. Dysregulation of BK channels has been implicated in hypertension (Gessner, G., Y. M. Cui, Y. Otani, T. Ohwada, M. Soom, T. Hoshi and S. H. Heinemann (2012); “Molecular mechanism of pharmacological activation of BK channels”, Proc Natl Acad Sci USA 109(9): 3552-3557; Bentzen et al, Frontiers in Physiology, Membrane Physiology and Membrane Biophysics, October 2014, Vol 15, Article 389, 1-12).

Abnormally elevated blood pressure is the most prevalent risk factor for cardiovascular disease. The large-conductance, voltage- and Ca²⁺⁻dependent (BK) channel has been proposed as an important effector in the control of vascular tone by linking membrane depolarization and local increases in cytosolic Ca²⁺ to hyperpolarizing K⁺ outward currents. Sausbier et al reported that deletion of the pore-forming BK channel alpha subunit leads to a significant blood pressure elevation resulting from hyperaldosteronism accompanied by decreased serum K⁺ levels as well as increased vascular tone in small arteries (Sausbier M. et at, Circulation, 2005 Jul. 5; 112(1):60-8, Epub 2005 May 2). In smooth muscle from small arteries, deletion of the BK channel leads to a depolarized membrane potential, a complete lack of membrane hyperpolarizing spontaneous K⁺ outward currents, and an attenuated cGMP vasorelaxation associated with a reduced suppression of Ca²⁺ transients by cGMP.

The BK channel therefore represents a therapeutic target for arterial hypertension, and pharmacological molecules that open the BK channel provide a promising treatment for this disorder.

BK channels have also been implicated in ischemic heart disease and psychoses (see Nardi and Olesen, Current Medicinal Chemistry 2008, 15, 1126-1146). The compounds described herein therefore have further therapeutic applications in the treatment of ischemic heart disease and psychoses.

In one preferred embodiment, the compounds described herein have therapeutic applications in the treatment of disorders associated with vascular dysfunction, particularly where this involves the endothelium, e.g. endothelial dysfunction.

In vascular diseases, endothelial dysfunction is a systemic pathological state of the endothelium (the inner lining of blood vessels) and can be broadly defined as an imbalance between vasodilating and vasoconstricting substances produced by (or acting on) the endothelium. Normal functions of endothelial cells include mediation of coagulation, platelet adhesion, immune function and control of volume and electrolyte content of the intravascular and extravascular spaces.

Endothelial dysfunction can result from and/or contribute to several disease processes, as occurs in hypertension, hypercholesterolaemia, diabetes, septic shock, and Behcet's disease, and it can also result from environmental factors, such as from smoking tobacco products and exposure to air pollution. Most of the studies on human participants have involved the percentage flow-mediated dilation (FMD %) index as the study outcome, which must have proper statistical consideration to be interpreted correctly. Endothelial dysfunction is a major physiopathological mechanism that leads towards coronary artery disease, and other atherosclerotic diseases.

In one preferred embodiment, the compounds described herein have therapeutic applications in the treatment of diseases of vascular dysfunction caused by obesity. Studies by Howitt et al demonstrated that dietary obesity abolished the contribution of large conductance Ca²⁺⁻activated K⁺ channels to ACh-mediated endothelium dependent dilation of rat cremaster muscle arterioles, while increasing NOS activity and inducing an NO-dependent component (Howitt, L, T. H. Grayson, M. J. Morris, S. L. Sandow and T. V. Murphy (2012). Am J Physiol Heart Circ Physiol 302(12): H2464-2476).

The compounds described herein also have therapeutic applications in the treatment of diabetes. Studies by Mori of al have demonstrated that vasodilation of retinal arterioles induced by activation of BK channels is attenuated in diabetic rats (Mori, A., S. Suzuki, K. Sakamoto, T. Nakahara and K. Ishii (2011), Eur J Pharmacol 669(1-3): 94-99; see also Nardi and Olesen, Current Medicinal Chemistry 2008, 15, 1126-1146). Thus, treatment with BK channel openers such as the presently described compounds provides a new therapeutic treatment for diabetes.

In one highly preferred embodiment, the compounds described herein have therapeutic applications in the treatment of diabetic retinopathy. Diabetic retinopathy, also known as diabetic eye disease, is when damage occurs to the retina due to diabetes. It can eventually lead to blindness. It is an ocular manifestation of diabetes, a systemic disease, which affects up to 80 percent of all patients who have had diabetes for 10 years or more. In one highly preferred embodiment, the compounds described herein have therapeutic applications in the treatment of other ocular diseases, for example, those involving retinal neurodegeneration of the optic nerve. Examples of such ocular diseases include Age Related Macular Degeneration (AMD) and retinitis pigmentosa, a group of inherited dystrophies with a prevalence of 1 in 2500 to 7000.

Age-related macular degeneration (AMD or ARMD or macular degeneration), is a medical condition that usually affects older adults and results in a loss of vision in the centre of the visual field (the macula) because of damage to the retina. It occurs in “dry” and “wet” forms. It is a major cause of blindness and visual impairment in older adults, afflicting 30-50 million people globally. Macular degeneration can make it difficult or impossible to read or recognize faces, although enough peripheral vision remains to allow other activities of daily life.

In the dry (nonexudative) form, cellular debris called drusen accumulates between the retina and the choroid (the network of blood vessels supplying the retina with blood), causing atrophy and scarring to the retina. In the wet (exudative) form, which is more severe, blood vessels grow up from the choroid behind the retina which can leak exudate and fluid and also cause hemorrhaging.

Retinitis pigmentosa (RP) is an inherited, degenerative eye disease that causes severe vision impairment due to the progressive degeneration of the rod photoreceptor cells in the retina. This form of retinal dystrophy manifests initial symptoms independent of age; thus, RP diagnosis occurs anywhere from early infancy to late adulthood. Patients in the early stages of RP first notice compromised peripheral and dim light vision due to the decline of the rod photoreceptors. The progressive rod degeneration is later followed by abnormalities in the adjacent retinal pigment epithelium (RPE) and the deterioration of cone photoreceptor cells. As peripheral vision becomes increasingly compromised, patients experience progressive “tunnel vision” and eventual blindness. Affected individuals may additionally experience defective light-dark adaptations, nyctalopia (night blindness), and the accumulation of bone spicules in the fundus (eye).

The compounds described herein also have therapeutic applications in the treatment of chronic obstructive pulmonary disorder. Chronic obstructive pulmonary disease (COPD) is the name for a collection of lung diseases including chronic bronchitis, emphysema and chronic obstructive airways disease. Large conductance voltage- and calcium-activated potassium (BK) channels are highly expressed in airway smooth muscle (ASM). Studies have shown that systemic administration of the BK channel agonist rottlerin reduces methacholine-induced airway hyperreactivity (AHR) in OVA- and HDM-sensitized mice, with a 35-40% reduction in inflammatory cells and 20-35% reduction in Th2 cytokines in bronchoalveolar lavage fluid. Intravenous rottlerin reduces AHR within 5 minutes in OVA-asthma mice by 45% (P<0.01). Rottlerin increases BK channel activity in human ASM cells and reduces the frequency of acetylcholine-induced Ca²⁺ oscillations in murine ex vivo lung slices (Goldklang M. P., Perez-Zoghbi J. F., Trischler J., Nkyimbeng T., Zakharov S. I., Shiomi T., Zelonina T., Marks A. R., D'Armiento J. M., Marx S. O.; FASEB J. 2013 December; 27(12):4975-86. dol: 10.1096/fj.13-235176. Epub 2013 Aug. 30). These findings suggest that rottlerin, with both anti-inflammatory and ASM relaxation properties, may have benefit in treating asthma and COPD.

The compounds described herein also have therapeutic applications in the treatment of erectile dysfunction (Gessner, G., Y. M. Cui, Y. Otani, T. Ohwada, M. Soom, T. Hoshi and S. H. Heinemann (2012), “Molecular mechanism of pharmacological activation of BK channels”, Proc Natl Acad Sci USA 109(9): 3552-3557; Bentzen et al, Frontiers in Physiology, Membrane Physiology and Membrane Biophysics, October 2014, Vol 15, Article 389, 1-12). Studies by Werner et al showed erectile dysfunction in mice lacking BK channels (Werner, M. E. et at, J Physiol. 2005 Sep. 1; 567(Pt 2):545-56. Epub 2005 Jul. 14). Thus, treatment with BK channel openers such as the presently described compounds provides a new therapeutic treatment for erectile dysfunction.

The compounds of the invention also have applications in neuroprotection, for example, in treating stroke. The term “neuroprotection” refers to protection of a neural entity, such as a neuron, at a site of injury, for example, an ischemic injury, or traumatic injury (Gessner, G., Y. M. Cui, Y. Otani, T. Ohwada, M. Soom, T. Hoshi and S. H. Heinemann (2012), “Molecular mechanism of pharmacological activation of BK channels”, Proc Natl Acad Sci USA 109(9): 3552-3557; see also Nardi and Olesen, Current Medicinal Chemistry 2008, 15, 1126-1146).

BK activators have been suggested as treatments for neuroprotection (Gribkoff, V. K., Starrett J. E., et al (2001). “Targeting acute ischemic stroke with a calcium-sensitive opener of maxi-K potassium channels.” Nat Med 7(4): 471-477). During ischemic stroke, neurons at risk are exposed to pathologically high levels of intracellular calcium (Ca²⁺), initiating a fatal biochemical cascade. Studies have shown that openers of large-conductance, Ca²⁺-activated (maxi-K or BK) potassium channels can protect these neurons, thereby augmenting an endogenous mechanism for regulating Ca²⁺ entry and membrane potential. The novel fluoro-oxindole BMS-204352 (Maxipost) was shown to be a potent, effective and uniquely Ca²⁺-sensitive opener of maxi-K channels. In rat models of permanent large-vessel stroke, BMS-204352 provided significant levels of cortical neuroprotection when administered two hours after the onset of occlusion, but had no effect on blood pressure or cerebral blood flow.

The compounds of the invention also have therapeutic applications in cardioplegia and cardiopulmonary bypass. Cardioplegia and cardiopulmonary bypass may produce deleterious effects that can be ameliorated by BK channel activation. Cardioplegia is intentional and temporary cessation of cardiac activity, primarily for cardiac surgery. The most common procedure for accomplishing asystole is infusing cold cardioplegic solution into the coronary circulation. This process protects the myocardium, or heart muscle, from damage during the period of ischemia. To achieve this, the patient is first placed on cardiopulmonary bypass. This device, otherwise known as the heart-lung machine, takes over the functions of gas exchange by the lung and blood circulation by the heart. Subsequently the heart is isolated from the rest of the blood circulation by means of an occlusive cross-clamp placed on the ascending aorta proximal to the innominate artery. During this period of heart isolation the heart is not receiving any blood flow, and thus no oxygen for metabolism. As the cardioplegia solution distributes to the entire myocardium the ECG will change and eventually asystole will ensue. Cardioplegia lowers the metabolic rate of the heart muscle thereby preventing cell death during the ischemic period of time.

Studies by Clements et a/ demonstrated that Rottlerin (a potent BK channel opener) increases cardiac contractile performance and coronary perfusion through BK channel activation after cold cardioplegic arrest in isolated hearts (Clements, R. T., B. Cordeiro, J. Feng, C. Bianchi and F. W. Sellke (2011), Circulation 124(11 Suppl): S55-61).

Thus, treatment with BK channel openers such as the presently described compounds provides a new therapeutic approach in cardioplegia and cardiopulmonary bypass.

Another aspect of the invention relates to the use of a compound as defined above in the preparation of a medicament for treating a disorder associated with BK channel modulation.

As used herein the phrase “preparation of a medicament” includes the use of a compound of formula I directly as the medicament in addition to its use in a screening programme for further agents or in any stage of the manufacture of such a medicament.

Another aspect of the invention relates to method of treating a disorder associated with BK channel modulation, said method comprising administering a pharmacologically effective amount of a compound as defined above to a subject in need of thereof.

Pharmaceutical Compositions

Even though the compounds for use according to the present invention (including their pharmaceutically acceptable salts, esters and pharmaceutically acceptable solvates) can be administered alone, they will generally be administered in admixture with a pharmaceutical carrier, excipient or diluent, particularly for human therapy. The pharmaceutical compositions may be for human or animal usage in human and veterinary medicine.

Examples of such suitable excipients for the various different forms of pharmaceutical compositions described herein may be found in the “Handbook of Pharmaceutical Excipients, 2^{nd} Edition, (1994), Edited by A Wade and P J Weller.

Acceptable carriers or diluents for therapeutic use are well known in the pharmaceutical art, and are described, for example, in Remington's Pharmaceutical Sciences, Mack Publishing Co. (A. R. Gennaro edit. 1985).

Examples of suitable carriers include lactose, starch, glucose, methyl cellulose, magnesium stearate, mannitol and sorbitol. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s).

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate and sodium chloride.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

Salts/Esters

The compounds for use according to the invention can be present as salts or esters, in particular pharmaceutically acceptable salts or esters.

Pharmaceutically acceptable salts of the compounds for use in the invention include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. hydrohalic acids (such as hydrochloride, hydrobromide and hydroiodide), sulphuric acid, phosphoric acid sulphate, bisulphate, hemisulphate, thiocyanate, persulphate and sulphonic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with amino acids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid.

Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminium hydroxide. Alcohols include alkanealcohols of 1-12 carbon atoms which may be unsubstituted or substituted, e.g. by a halogen).

Enantiomers/Tautomers

The compounds for use according to the invention include, where appropriate all enantiomers and tautomers of the compounds of formula I. The man skilled in the art will recognise compounds that possess optical properties (one or more chiral carbon atoms) or tautomeric characteristics. The corresponding enantiomers and/or tautomers may be isolated/prepared by methods known in the art. Thus, the invention encompasses the enantiomers and/or tautomers in their isolated form, or mixtures thereof, such as for example, racemic mixtures of enantiomers.

Stereo and Geometric Isomers

Some of the specific agents of the invention may exist as stereoisomers and/or geometric isomers—e.g. they may possess one or more asymmetric and/or geometric centres and so may exist in two or more stereoisomeric and/or geometric forms. The present invention contemplates the use of all the individual stereoisomers and geometric isomers of those agents, and mixtures thereof. The terms used in the claims encompass these forms, provided said forms retain the appropriate functional activity (though not necessarily to the same degree).

The present invention also includes the use of all suitable isotopic variations of the agent or a pharmaceutically acceptable salt thereof. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the agent of the present invention and pharmaceutically acceptable salts thereof of this invention can generally be prepared by conventional procedures using appropriate isotopic variations of suitable reagents.

Solvates

The present invention also includes the use of solvate forms of the compounds of the present invention. The terms used in the claims encompass these forms. Preferably, the solvate is a hydrate.

Polymorphs

The invention furthermore relates to the use of compounds of the present invention in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

Prodrugs

The invention further includes the use of compounds of the present invention in prodrug form. Such prodrugs are generally compounds of formula I wherein one or more appropriate groups have been modified such that the modification may be reversed upon administration to a human or mammalian subject. Such reversion is usually performed by an enzyme naturally present in such subject, though it is possible for a second agent to be administered together with such a prodrug in order to perform the reversion in vivo. Examples of such modifications include ester (for example, any of those described above, for example, methyl or ethyl esters of the acids), wherein the reversion may be carried out be an esterase etc. Other such systems will be well known to those skilled in the art.

In one highly preferred embodiment, the prodrug is an ester of said compound of formula I, more preferably a methyl or ethyl ester. For example, the free COOH group of the compound of formula I is esterified to form a COOR¹¹ group, where R¹¹ is a C₁₋₆-alkyl group.

Administration

The pharmaceutical compositions for use in accordance with the present invention may be adapted for oral, rectal, topical, vaginal, parenteral, intramuscular, intraperitoneal, intraarterial, intrathecal, intrabronchial, subcutaneous, intradermal, intravenous, nasal, buccal or sublingual routes of administration.

For oral administration, particular use is made of compressed tablets, pills, tablets, gellules, drops, and capsules. Preferably, these compositions contain from 1 to 250 mg and more preferably from 10-100 mg, of active ingredient per dose.

Other forms of administration comprise solutions or emulsions which may be injected intravenously, intraarterially, intrathecally, subcutaneously, intradermally, intraperitoneally or intramuscularly, and which are prepared from sterile or sterilisable solutions. The pharmaceutical compositions of the present invention may also be in form of suppositories, pessaries, suspensions, emulsions, lotions, ointments, creams, gels, sprays, solutions or dusting powders.

Other forms of administration comprise solutions or emulsions which are in a form suitable for ocular delivery, for example, eye drops, gels, ointments, sprays, creams or specialist ocular delivery devices.

An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

Injectable forms may contain between 10-1000 mg, preferably between 10-250 mg, of active ingredient per dose.

Compositions may be formulated in unit dosage form, i.e., in the form of discrete portions containing a unit dose, or a multiple or sub-unit of a unit dose. In addition, the compositions may be formulated as extended release formulations.

Dosage

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

Depending upon the need, the agent may be administered at a dose of from about 0.01 to about 30 mg/kg body weight, such as from about 0.1 to about 10 mg/kg, more preferably from about 0.1 to about 1 mg/kg body weight. In one highly preferred embodiment, the dose is from about 2 to about 6 mg/kg body weight, more preferably, about 5 mg/kg body weight.

In an exemplary embodiment, one or more doses of 10 to 150 mg/day will be administered to the patient.

Combinations

In a particularly preferred embodiment, the one or more compounds of the invention are for use in combination with one or more other pharmaceutically active agents. In such cases, the compounds of the invention may be administered consecutively, simultaneously or sequentially with the one or more other pharmaceutically active agents.

The present invention is further described by way of example, and with reference to the following figures wherein:

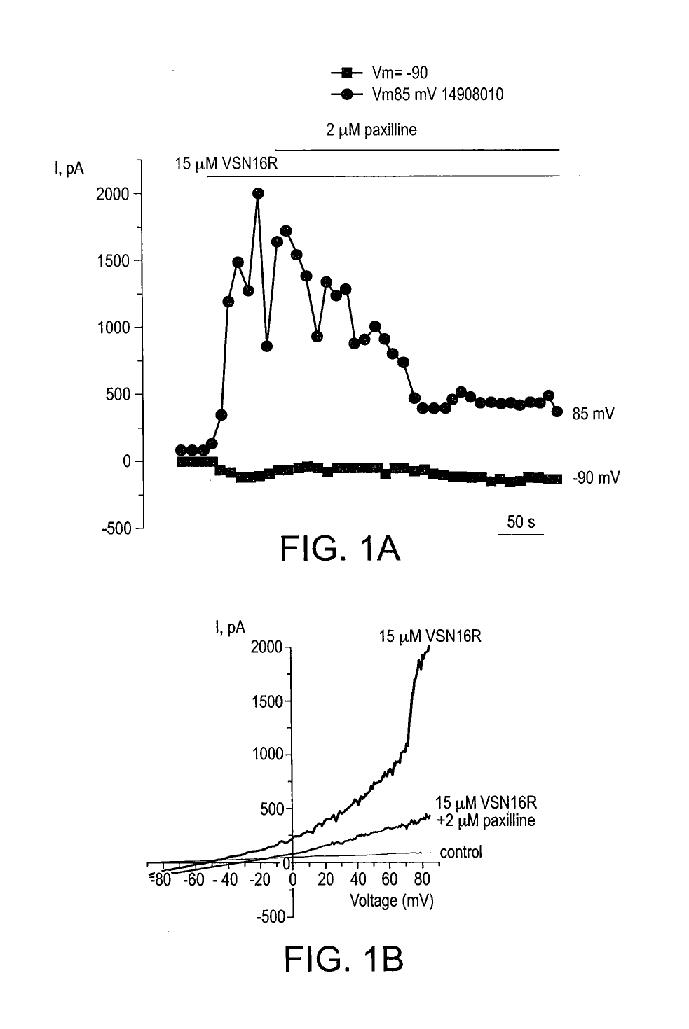

FIG. 1 shows the effect of VSN16R on whole-cell BK current in human EA.hy926 cells. VSN16R induces currents that are sensitive to paxilline as shown in plots current vs time (A) and current vs voltage (B).

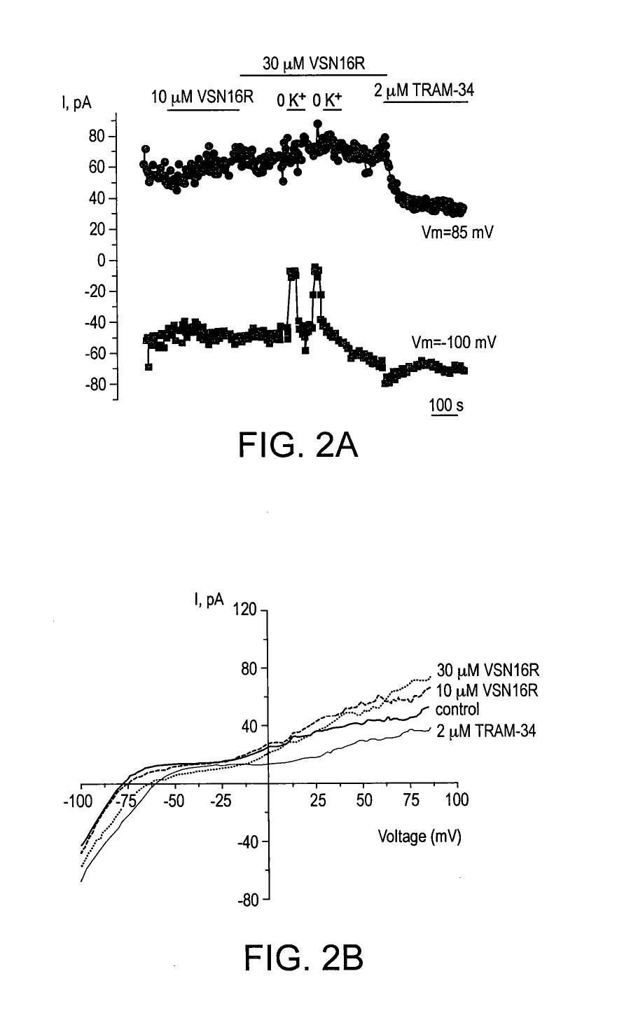

FIG. 2 shows that cells which do not express BK beta chains are insensitive to VSN16R (current vs time (A) and current vs voltage (B)). Pig aortic endothelial cells VSN16R shows only a minor effect on current. TRAM-34 (a blocker of IK channels) shows a minor effect on this residual current.

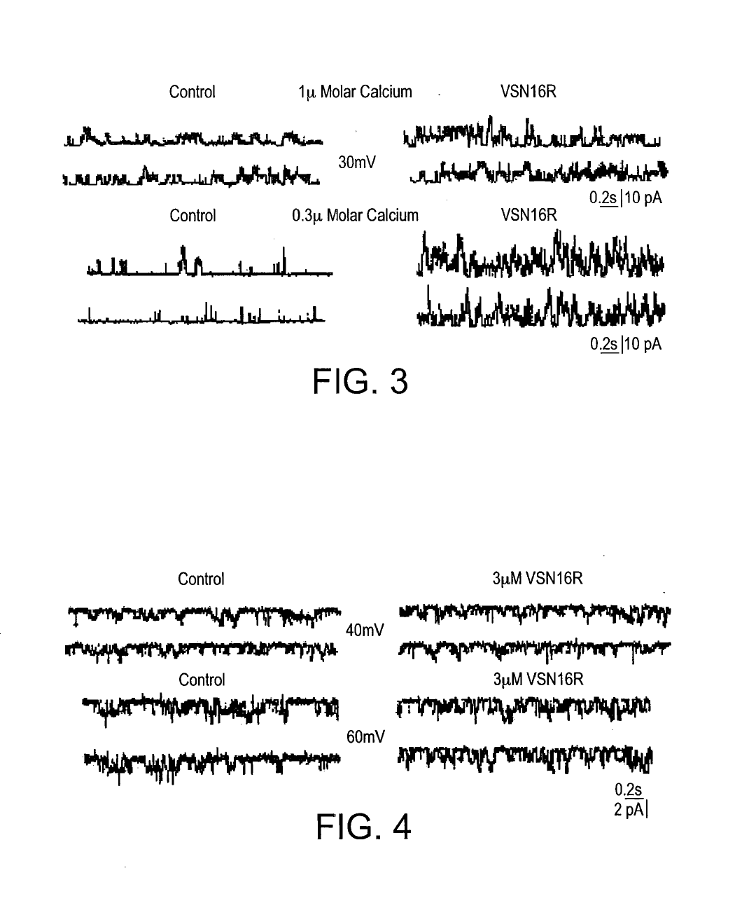

FIG. 3 shows inside-out patch clamp studies on human EA.hy926 cells. Treatment of the cells with VSN16R gives an activation of the response of the channel and the effect is notably calcium dependent.

FIG. 4 shows inside-out patch clamp of pig aortic endothelial cells. VSN16R does not give a response in these cells.

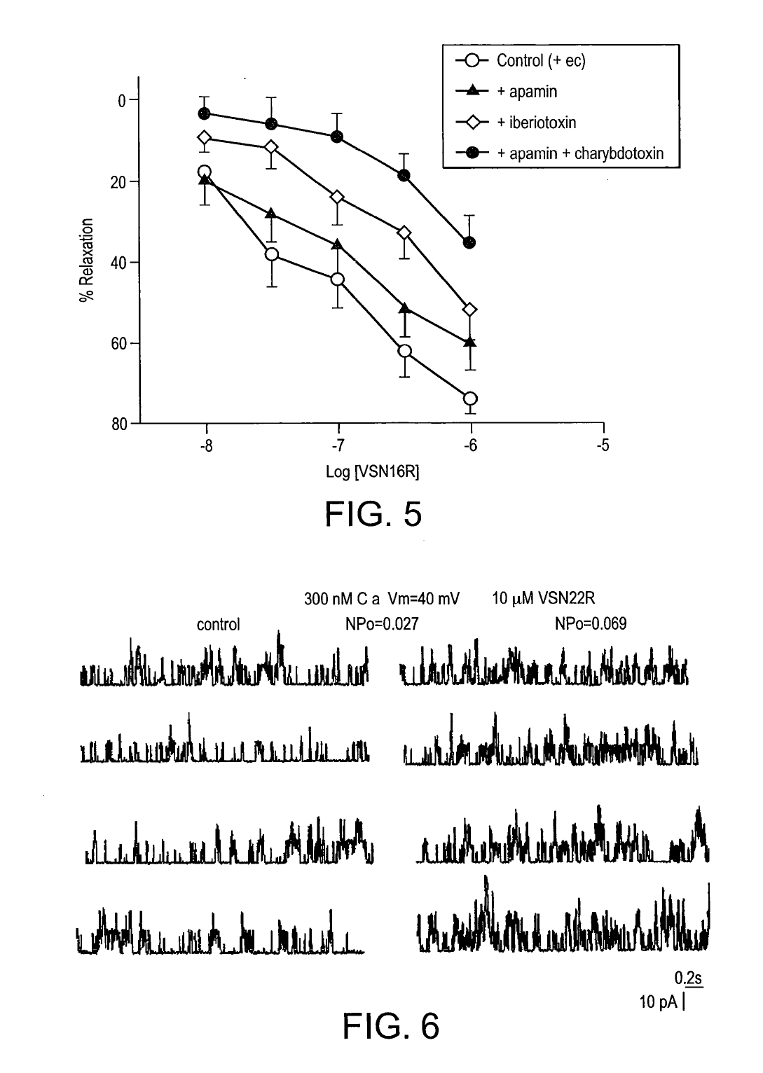

FIG. 5 shows VSN16R induced relaxation of rat mesenteric arteries is sensitive to BKCa blockade. Rat mesenteric arteries pre-contracted with methoxamine are relaxed by VSN16R. This relaxation is blocked by iberotoxin, and the combination of apamin (SK blocker) and charybdotoxin (non-selective potassium channel blocker. Apamin alone gives a non-significant blockade. Addition of 60 mM KCl blocks K⁺ channels and depolarises the cells inhibiting VSN16R activity.

FIG. 6 shows inside-out patch clamp studies on human EA.hy926 cells. Treatment of the cells with VSN22R gives an activation of the response of the channel and the effect is notably calcium dependent.

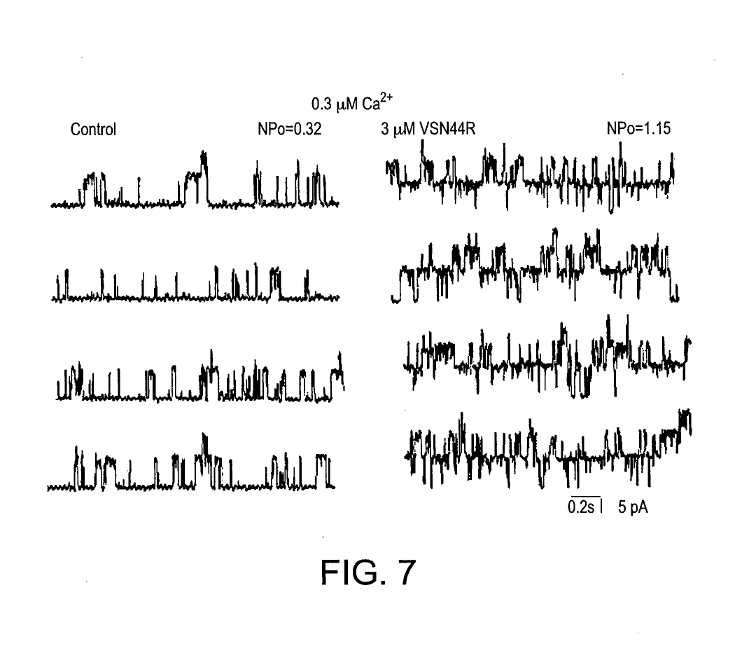

FIG. 7 shows inside-out patch clamp studies on human EA.hy926 cells. Treatment of the cells with VSN44R gives an activation of the response of the channel and the effect is notably calcium dependent.

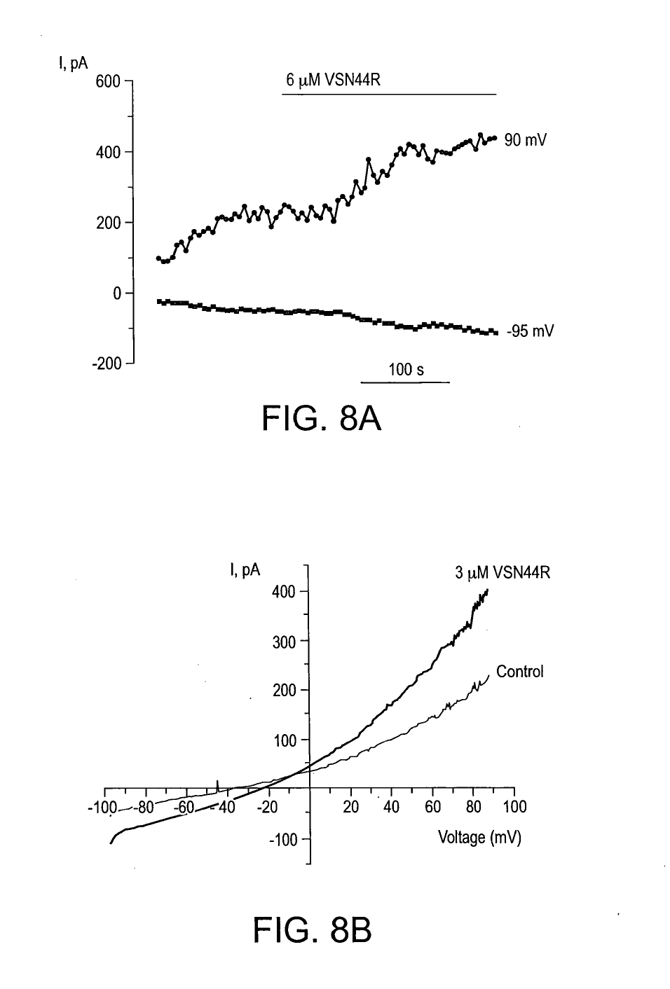

FIG. 8 shows the effect of VSN44R on whole-cell BKCa current in human EA.hy926 cells, in current vs time (A) and current vs voltage (B).

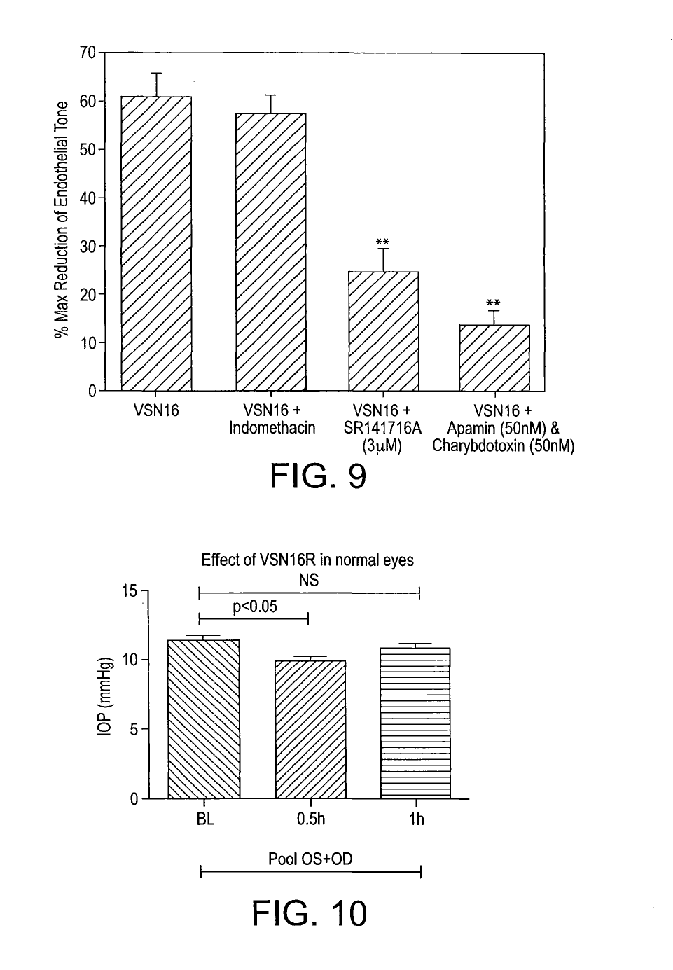

FIG. 9 shows that VSN16R activates calcium activated potassium channels in an arterial vasodilation (smooth muscle relaxation) assay. More specifically, FIG. 9 shows maximum reduction of endothelial tone against different treatment groups (i) VSN16, (ii) VSN+Indomethacin, (iii) VSN+SR141716A, and (iv) VSN+Apamin+Charybdotoxin.

FIG. 10 shows that VSN16R significantly reduces IOP (mmHg) at 0.5 h (mean 9.82) (p<0.05), but not 1 h (10.79), compared to BL (11.18);

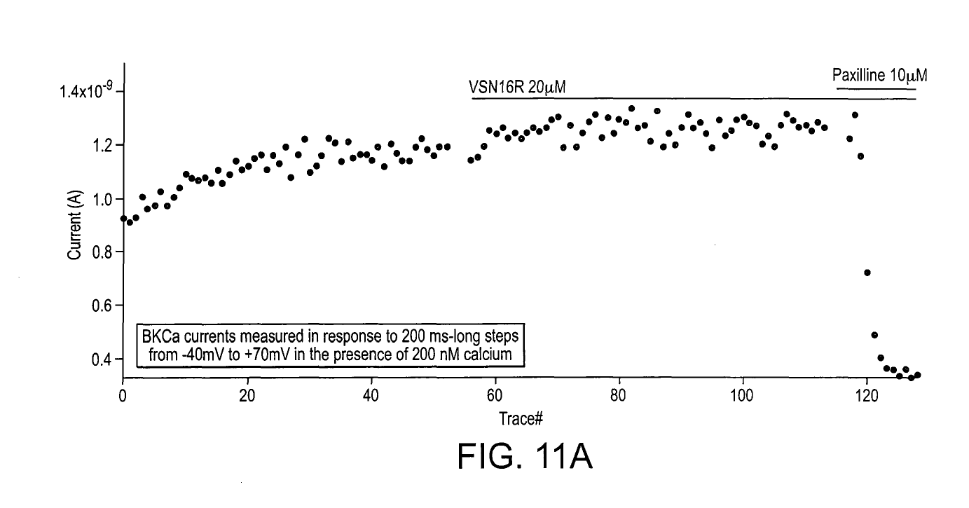

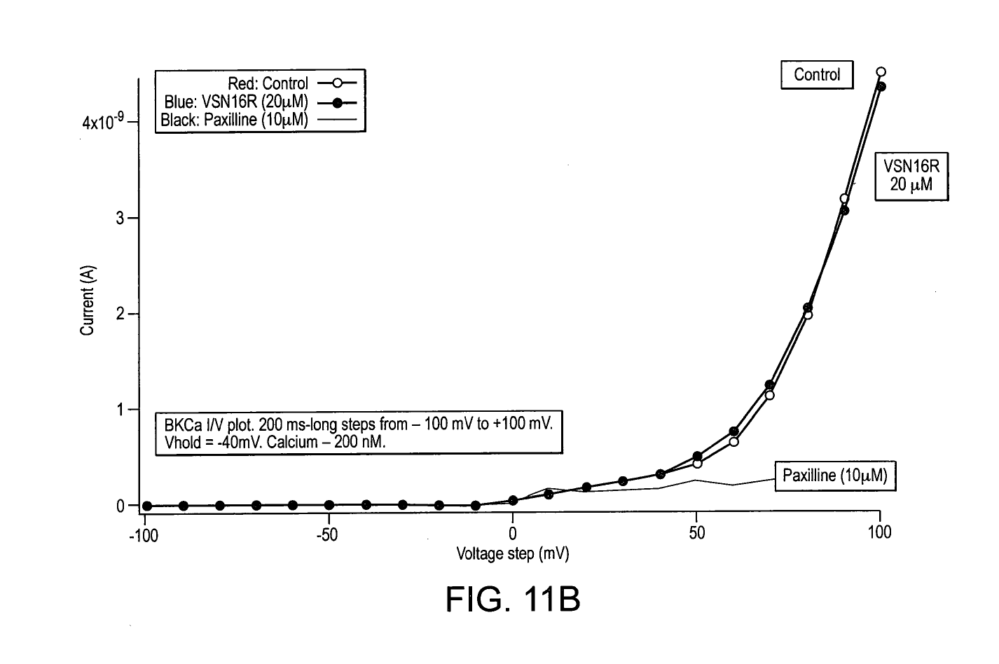

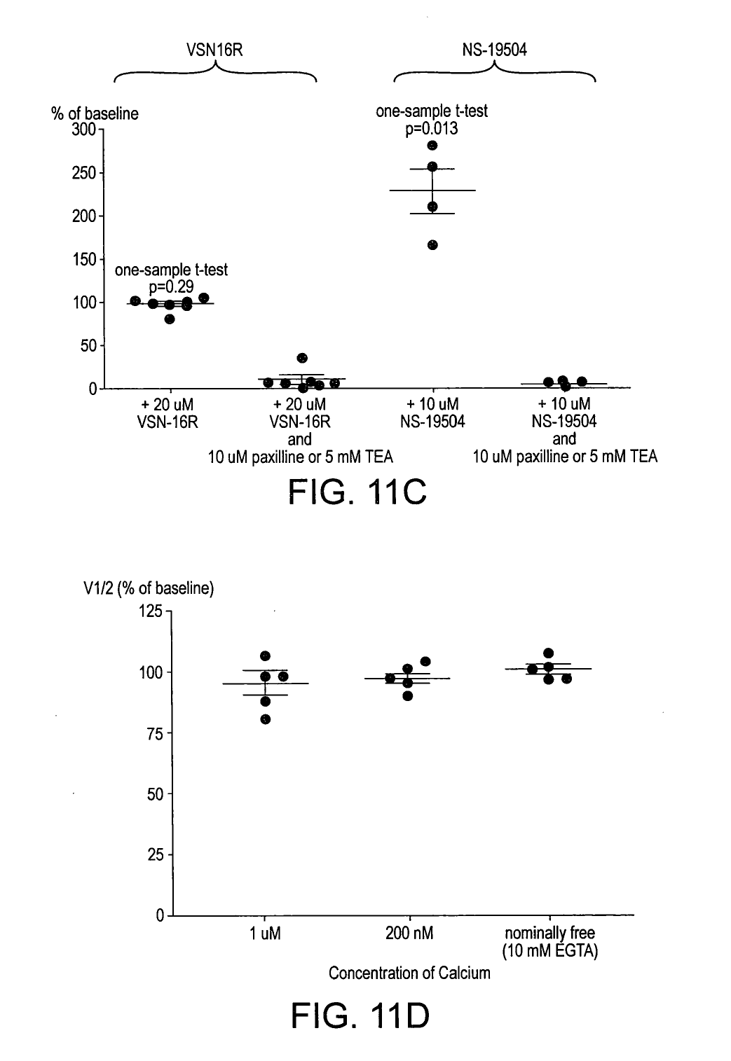

FIG. 11 shows the effect of VSN16R on whole-cell BKCa current in HEK293 cells; FIG. 11A shows current against time for the action of VSN16R (20 μM) on BKCa currents measured in the whole-cell configuration and elicited by 200 ms-long voltage steps from −40 mV to +70 mV in the presence of 200 nM calcium; FIG. 11B shows current against voltage relationship for BKCa currents measured under control conditions, in the presence of the BKCa opener VSN16R (20 μM), and in the presence of paxilline (10 μM); FIG. 11C shows the relative enhancement of BKC currents caused by VSN16R and the non-selective BKCa opener NS19504 in seven different cells; FIG. 11D shows the effect of 20 μM VSN16R on the activation voltage of BKCa current when applied in the presence of various concentrations of intracellular calcium (1 μM, 200 nM; nominally 0M).

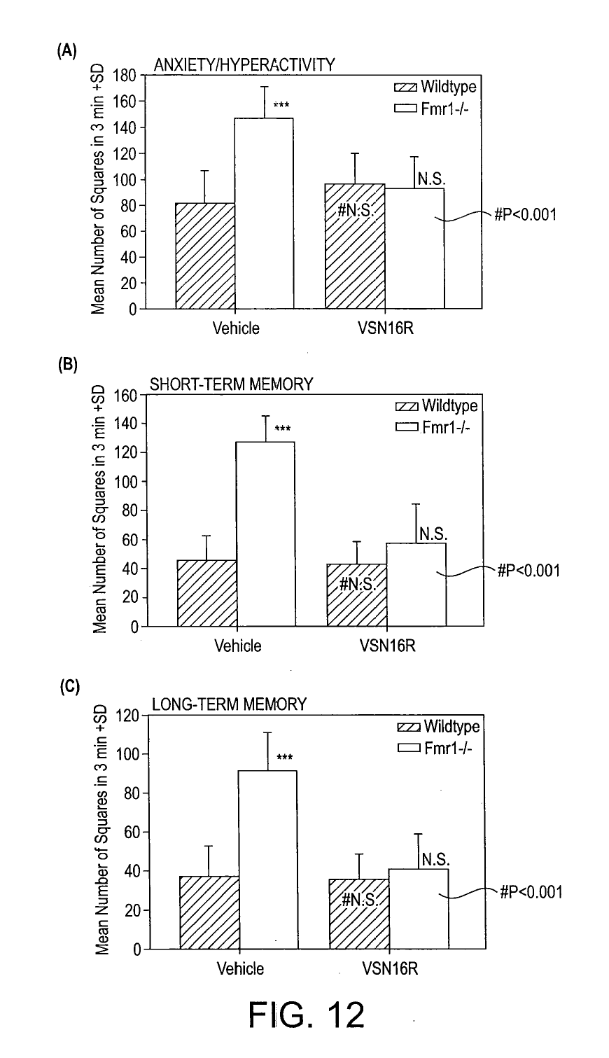

FIG. 12 shows that VSN16R inhibits the hyperactivity and memory deficient present in Fmr1 knockout mice. C57BL/6 wildtype and C57BL/6.Fmr1 knockout mice were placed in an open field chamber at (A) baseline and again (B) 10 minutes and (C) 24 hours later. The activity was recorded over 3 minuutes as assessed by the number of 10×10 cm square areas within the open field chamber following either treatment with 0.1 ml PBS vehicle or 2mg/kg i.v. VSN16R. N=10 per group. The results represent the mean±SD. **P<0.001 compared to wildtype mice. # compared to vehicle treatment mice.

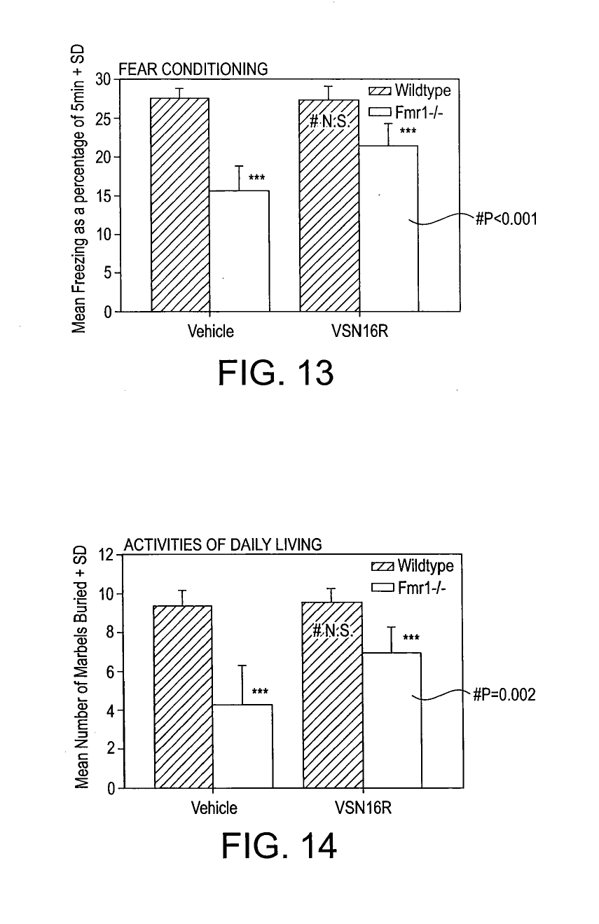

FIG. 13 shows that VSN16R inhibits the development of fear conditioning present in Fmr1 knockout mice. More specifically, C57BL/6 wildtype and C57BL/6.Fmr1 knockout mice were conditioned to associate electroshock to a fear conditioning environment and the amount of time spent freezing in the conditioned environment was assessed following either treatment with 0.1 ml PBS vehicle or 2 mg/kg i.v. VSN16R. The results represent the mean±SD. N=10 per group. **P<0.001 compared to wildtype mice. # compared to vehicle treatment mice n=10/ group.

FIG. 14 shows that VSN16R inhibits the development of Exaggerated Digging Behaviours present in Fmr1 knockout mice. Marbles were placed within the cages of C57BL/6 wildtype and C57BL/6.Fmr1 knockout mice. The number of marbles buried under the sawdust was assessed 30 min This was assessed following either treatment with 0.1 ml PBS vehicle or 2 mg/kg i.v. VSN16R. The results represent the mean±SD. N=10/ group **P<0.001 compared to wildtype mice. # compared to vehicle treatment mice.

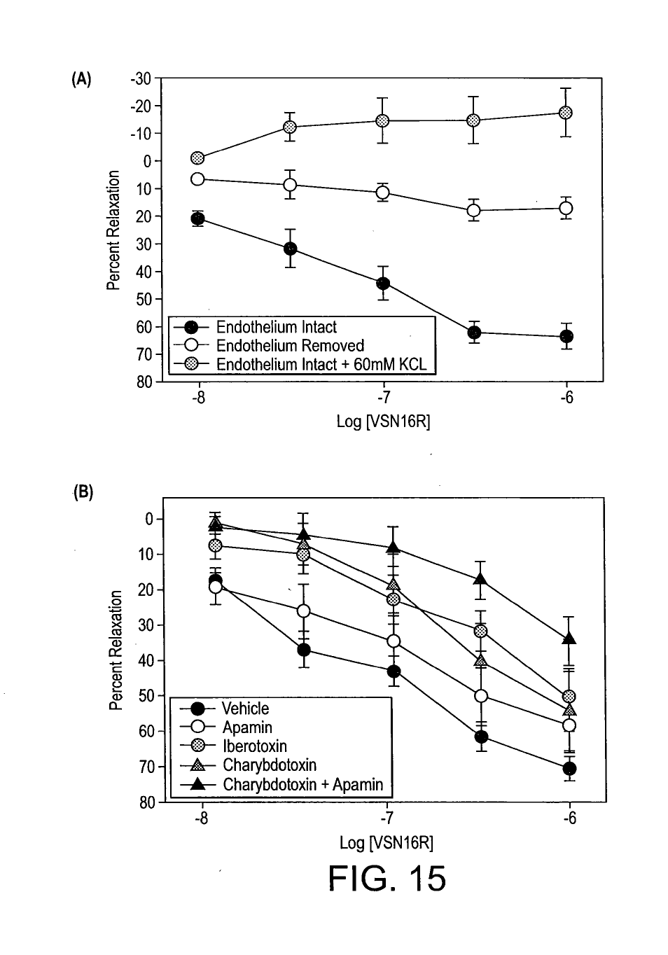

FIG. 15 shows the effect of VSN16 on the relaxation of rat mesenteric arteries (percent relaxation versus log[VSN16]): (A) endothelial intact (n=11), endothelium denuded (n=11) cultures or endothelium intact cultures in the presence of 60 mM KCl (n=6); (B) vehicle-treated controls (n=17 animals) or pretreated with 50 nM apamin (n=7), 50 nM iberotoxin (n=5), 50 nM charybdotoxin (n=6) or a combination of apamin and charybdotoxin (n=6).

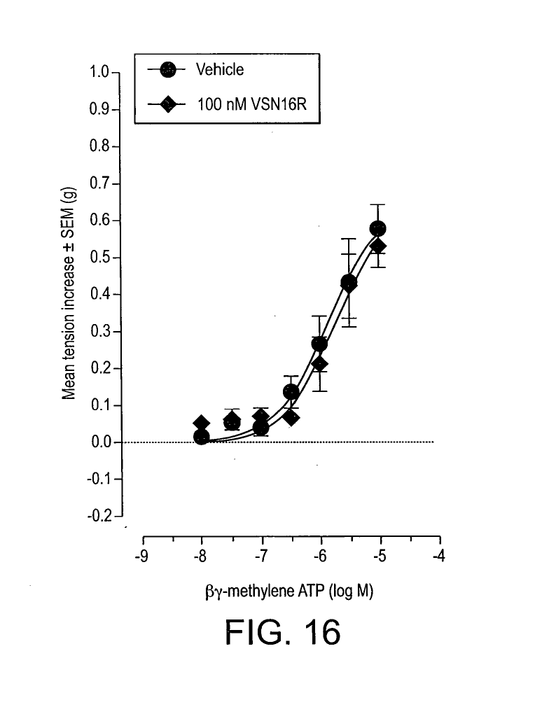

FIG. 16 shows the effect of VSN16R on beta gamma methylene adenosine triphosphatase-induced muscle contraction in the vas deferens. Mouse vas deferens were treated with either DMSO vehicle or 100 nM VSN16R 30 min before the first organ bath injection of various concentrations of βγ-methylene ATP into the organ bath. The results represent the mean±SEM of βγ-methylene ATP-induced increases in tension (expressed in grams) of electrically unstimulated vasa deferentia. (n=6/ group). Vehicle EC₅₀=1347 nM, Vehicle VSN16R=1832 nM (95%Cl 836-40 11 nM).

### EXAMPLES

General Procedures

All starting materials and solvents were obtained either from commercial sources or prepared according to the literature citation. Unless otherwise stated all reactions were stirred.

Normal phase column chromatography was routinely carried out on an automated flash chromatography system such as CombiFlash Companion or CombiFlash RF system. Intermediates were purified using pre-packed silica (230-400 mesh, 40-63 μm) cartridges and products of a Lindlar reduction using pre-packed GraceResolv flash cartridges. SCX was purchased from Supelco or Silicycle (40-63 μm size, 0.78 mmol/g loading).

Analytical Methods

Analytical HPLC was carried out using a Waters Xselect CSH C18, 2.5 μm, 4.6×30 mm column eluting with a gradient of 0.1% Formic Acid in MeCN in 0.1% aqueous Formic Acid; a Waters Xbridge BEH C18, 2.5 μm, 4.6×30 mm column eluting with a gradient of MeCN in aqueous 10 mM Ammonium Bicarbonate. UV spectra of the eluted peaks were measured using either a diode array or variable wavelength detector on an Agilent 1100 system.

Analytical LCMS was carried out using a Waters Xselect CSH C18, 2.5 μm, 4.6×30 mm column eluting with a gradient of 0.1% Formic Acid in MeCN in 0.1% aqueous Formic Acid; a Waters Xbridge BEH C18, 2.5 μm, 4.6×30 mm column eluting with a gradient of MeCN in aqueous 10 mM Ammonium Bicarbonate. UV and mass spectra of the eluted peaks were measured using a variable wavelength detector on either an Agilent 1200 with or an Agilent Infinity 1260 LCMS with 6120 single quadrupole mass spectrometer with positive and negative ion electrospray.

Preparative HPLC was carried out using a Waters Xselect CSH C18, 5 μm, 19×50 mm column using either a gradient of either 0.1% Formic Acid in MeCN in 0.1% aqueous Formic Acid or a gradient of MeCN in aqueous 10 mM Ammonium Bicarbonate; or a Waters Xbridge BEH C18, 5 μm, 19×50 mm column using a gradient MeCN in aqueous 10 mM Ammonium Bicarbonate; or the compounds were purified by reverse-phase HPLC (Gilson) using preparative C-18 column (Hypersil PEP 100×21 mm internal diameter, 5 μm particle size, and 100 Å pore size) and isocratic gradient over 20 minutes. Fractions were collected following detection by UV at a single wavelength measured by a variable wavelength detector on a Gilson 215 preparative HPLC or Varian PrepStar preparative HPLC; by mass and UV at a single wavelength measured by a ZQ single quadrupole mass spectrometer, with positive and negative ion electrospray, and a dual wavelength detector on a Waters FractionLynx LCMS. ¹H NMR Spectroscopy: ¹H NMR spectra were acquired on a Bruker Avance Ill spectrometer at 400 MHz. The central peak of dimethylsulfoxide-d₆ was used as reference.

### ABBREVIATIONS

AcOH glacial acetic acid

aq. aqueous

br broad

d doublet

dd doublet of doublets

ddd double double doublet

dt doublet of triplets

DCM dichloromethane

DIPEA diisopropylethylamine

DMF dimethylformamide

DMSO dimethyl sulfoxide

(ES+) electrospray ionization, positive mode

Et ethyl

Et₃N triethylamine

EtOAc ethyl acetate

EtOH ethanol

HATU 2-(1H-7-azabenzotriazol-1-yl)-1,1,3,3-tetramethyluronium hexafluorophosphate

HCl hydrochloric acid

HPLC high performance liquid chromatography

hr hour(s)

Hz hertz

LC liquid chromatography

(M+H)+ protonated molecular ion

M molar

m multiplet

Me methyl

MeCN acetonitrile

MeOH methanol

MgSO₄ magnesium sulphate

MHz megahertz

min minute(s)

MS mass spectrometry

m/z: mass-to-charge ratio

Na₂SO₄ sodium sulphate

NMR nuclear magnetic resonance (spectroscopy)

Ph phenyl

ppm parts per million

q quartet

qn quintet

rt room temperature

HPLC high performance liquid chromatography

s singlet

sat. saturated

SCX solid supported cation exchange (resin)

t triplet

td triplet of doublets

TEA triethylamine

THF tetrahydrofuran

TLC thin layer chromatography

wt % weight percent

Prefixes n-, s-, i-, t- and tert- have their usual meanings: normal, secondary, iso, and tertiary.

Compounds for use according to the present invention may be prepared in accordance with the methods described in WO 2005/080316, WO 2010/116116 and WO 2015/082938.

Paxilline has the chemical name_(2R,4bS,6aS,12bS,12cR,14aS)-5,6,6a,7,12,12b,12c,13,14,14a-Decahydro-4b-hydroxy-2-(1-hydroxy-1-methylethyl)-12b,12c-dimethyl-2H-pyrano[2″,3″:5′,6′]benz[1′,2′:6,7]indeno[1,2-b]indol-3(4bH)-one (CAS 57186-25-1). NS19054 is the compound 5-[(4-bromophenyl)methyl]-2-thiazolannine (CAS 327062-46-4). Paxilline and NS19054 are commercially available from a number of sources, including Tocris Bioscience and Alomone Labs.

General Method for Amide Coupling:

To a suspension of carboxylic acid (1.0 eq.), amine or amine.HCl salt (1.05-1.1 eq.) and HATU (1.1-1.3 eq.) in dry DCM (10 mL/g) was added DIPEA or TEA (2.0-3.0 eq.). The reaciton was stirred at rt until complete by LCMS. The volatiles were removed in vacuo and the residue partitioned between EtOAc (20 mL/g) and sat. aq. ammonium chloride (20 mL/g). The aqueous layer was extracted with EtOAc (2×20 mL/g) before the combined organic extracts were washed with sat. aq. ammonium chloride (30 mL/g), water (30 mL/g) then brine (30 mL/g) and dried (MgSO₄ or Na₂SO₄), filtered and concentrated in vacuo. The crude material was purified by column chromatography.

General Method for Sonogashira Coupling:

To a solution of aryl iodide (1.0 eq.) and diisopropylamine (1.2 eq.) in dry THF (10 mL/g) under nitrogen was added bis(triphenylphosphine)palladium(II) chloride (4 mol %) and copper(I) iodide (7 mol %). The reaction was stirred for 5 min before alkyne (1.1-1.5 eq.) was added. The reaction was then heated at 60° C. for 1 h before the solvent was removed in vacuo and the residue partitioned between EtOAc (20 mL/g) and sat. aq. ammonium chloride (20 mL/g). The aq. layer extracted with EtOAc (2×20 mL/g) before the combined organic extracts were washed with sat. aq. ammonium chloride (20 mL/g), water (20 mL/g) and brine (20 mL/g) then dried (MgSO₄), filtered and concentrated. The crude material purified by column chromatography to yield desired coupled product.

General Method for Lindlar Reduction:

To a flask containing palladium on barium sulphate reduced (5%) (50 wt % cf. alkyne) under nitrogen was added a solution of alkyne (1.0 eq.) and quinoline (1.3 eq.) in MeOH (40 mL/g). The vessel was placed under an atmosphere of hydrogen until the reaction was deemed complete by TLC, HPLC or LCMS analysis. The catalyst was removed by filtration through celite and the quinoline was removed by filtration through SCX (washing several times with MeOH). The filtrate

General Method for Ester Saponification:

To a solution of ester (1.0 eq.) in THF (10 mL/g) was added a solution of lithium hydroxide (1.5-2.0 eq.) in water (1 mL/g). The reaction was stirred at rt until judged complete by HPLC or LCMS analysis. The volatiles were removed in vacua and the residue was partitioned with EtOAc (10 mL/g). The aqueous layer was acidified to pH 1 with 1 N citric acid and extracted with EtOAc (3×10 mL/g). The combined organic extracts were washed with water (2×10 mL/g) and brine (10 mL/g) then dried (Na₂SO₄), filtered and concentrated in vacuo.

General Procedure for Reduction of Alkyne to Alkane:

To a flask containing alkyne (1.0 eq.) in EtOH (15-20 mL/g) under nitrogen was added palladium on carbon (5 wt %) (50 wt % cf. alkyne). The mixture was placed under an atmosphere of hydrogen (2 bar) until judged complete by LCMS analysis. The catalyst was removed by filtration through celite and washed well with EtOH. The filtrate was then concentrated in vacuo and purified by chromatography to give the desired alkane product.

Preparation of VSN-44

The compound VSN-44 can be prepared by the following methodology. Other compounds of formula I can be prepared by analogous methodology using commercially availably starting materials and standard synthetic steps that would be familiar to the skilled artisan, including those set forth in WO 2005/080316 and WO 2010/116116.

3-[(1Z)-6-(dimethylamino)-6-oxohex-1-en-1-yl] benzoic acid (IIb)

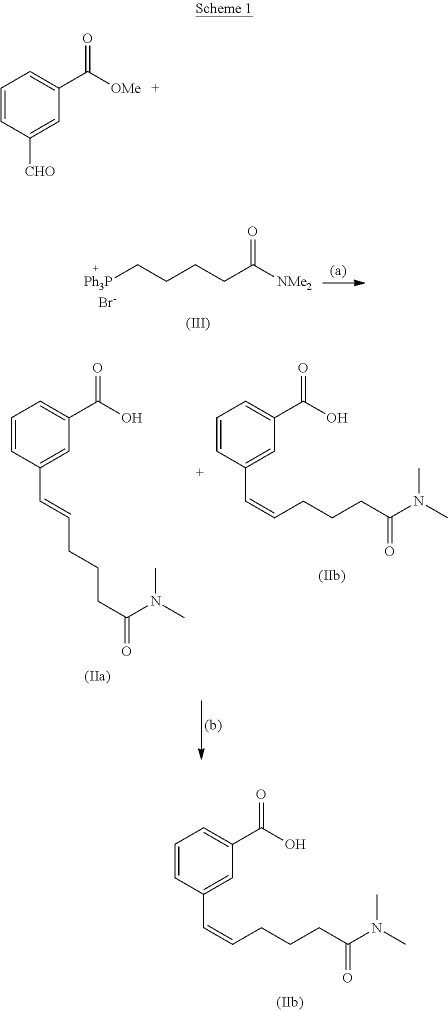

Scheme 1: (a) (i) anhydrous CH₂Cl₂, potassium hexamethyl disilazide, THF under N₂ atmosphere, <10° C.; (ii) NaOH, MeOH; (b) (i) DMAP (EtOAc, Et₂O); (ii) separation of isomers.

N,N-dimethylamino 4-(carboxybutyl) triphenylphosphonium bromide (III) 4-(carboxybutyl)triphenylphosphonium bromide (140 g, 0.315 mol, 1 equiv) was charged in a reactor and dichloromethane (650 ml, 4.5 vols) was added. Triethylamine (dried on molecular sieves; 95 ml, 2.1 equiv) was charged and the reaction mixture was cooled down to −10° C. Ethyl chloroformate (40 ml, 1.05 equiv) was added dropwise and the mixture was stirred for another 15 min at −10° C.

A solution containing dimethylamine hydrochloride (freshly crystallised from methanol/ether; 78 g, 3 equiv) and triethylamine (200 ml, 4.5 equiv) in dichloromethane (1000 ml, 7 vols) was prepared.

This solution was stirred for 40 min at room temperature and added dropwise to the reaction mixture at −10° C. The temperature was kept between −10 and −15° C. during all the addition. The reaction was left to warm up to room temperature. The reaction was stirred at room temperature overnight. The mixture was treated with 2 l of saturated NaHCO₃ solution. The aqueous phase was extracted with dichloromethane (1×2 l and 2×1 l). Organics were combined and dried over MgSO₄ and filtered. The volatiles were removed under vacuum. The residue was triturated with 350 ml of diethyl ether. The solid was filtered and triturated with hot diethyl ether for 5 hours. The suspension was cooled down and the solid filtered. The solid was dried under vacuum to give 130.9 g of a white solid (III) (90% yield).

¹H NMR (CDCl₃) 7.65-8.0 (m, 15H); 3.7 (m, 2H); 3.0 (s, 3H); 2.8 (s, 3H), 2.5 (t, J=7 Hz, 2H); 1.9 (m, 2H), 1.7 (m, 2H).

### 3-[(1Z)-6-(dimethylamino)-6-oxohex-1-en-1-yl] benzoic acid (IIb)

N,N-dimethylamino 4-carboxybutyltriphenylphosphonium (III) (61.9 g, 0.13 mol, 3 equivalents) were dissolved in dry dichloromethane (150 ml, 2.4 vols) under nitrogen. The solution was cooled down to 0° C. and potassium hexamethyldisilazide (0.9M in THF; 45 ml, 5 equiv) was added dropwise at 0° C. The reaction mixture was stirred at 0° C. for another 45 min. A solution of methyl 3-formylbenzoate (7.16 g, 1 equiv) in dry THF (36 ml, 5 vols) was added keeping the temperature <4° C. The mixture was allowed to warm up to room temperature and was stirred for 18 hrs. The reaction was quenched with 2M HCl (400 ml) and extracted with dichloromethane (2×400 ml and 2×200 ml). Organics were combined, dried over MgSO₄, filtered and evaporated to dryness. The residue was dissolved in a mixture of sodium hydroxide 1M/methanol 4:1 (440 ml) and stirred for 18 hrs. Water (100 ml) was added to the mixture and methanol was evaporated under vacuum. Aqueous was extracted with ethyl acetate (400 ml). The pH was adjusted to pH 1 and the mixture was extracted with dichloromethane (2×400 ml and 2×200 ml). Organics were dried over MgSO₄, filtered and evaporated to dryness. M=22.0 g. The crude was purified by flash chromatography using dichloromethane to dichloromethane/MeOH=95/54 as eluent. M=10.6 g 93% yield.

Isomer Separation

Acid (10.93 g, 0.042 mol) was dissolved in ethyl acetate (20 ml) and 4-dimethylaminopyridine (6.13 g, 1.2 equiv) was dissolved in warm ethyl acetate (20 ml). The DMAP solution was added to the free acid solution. The mixture was stirred at reflux temperature for 10 min. Then, the solution was allowed to cool down to room temperature slowly. A brown salt was formed, which was removed by filtration. A mixture of diethyl ether/ethyl acetate: 9:1 (40 ml) was added and the solution was heated to reflux. The mixture was stirred and allowed to cool down overnight. A pale yellow solid was filtered and dried in-vacua. This solid was treated with HCl (1M) and extracted with dichloromethane (3×50 ml). Organics were dried over MgSO₄, filtered and evaporated to dryness to give a brown oil which solidified upon standing (IIb). M=3.88 g (35.5% yield).

¹H NMR (CDCl₃) 9.7 (bs, 1H); 8.0 (m, 2H); 7.5 (m, 2H); 6.5 (d, J=11 Hz, 1H); 5.75 (m, 1H); 3.0 (s, 6H); 2.4 (m, 4H); 1.9 (m, 2H)

Preparation of VSN44

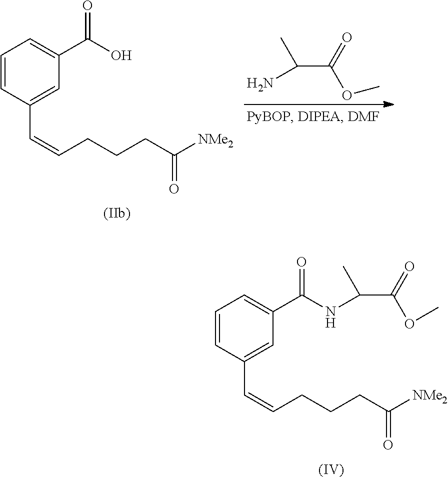

To the substituted benzoic acid (IIb) (139 mg, 1 mmol) in DMF (1 mL) was added the Ala(OMe) in DMF (1 mL) and the PyBOP (572 mg, 1.1 mmol) added in DMF (2 mL). DIPEA (142 mg, 191 μL, 1.1 mmol) was added dropwise, and the reaction stirred at room temperature overnight. Water (50 mL) was added and ethyl acetate (100 mL). The layers were stirred (5 mins), separated, and the ethyl acetate layer washed with brine (2×100 mL), dried (Na₂SO₄) to give the crude product (650 mg). This was flash chromatographed using a 25 g Puriflash (silica) column, cyclohexane:acetone 15-45% gradient. Yield (IV) 180 mg, 0.54 mmol, 54%.

1H NMR (500 MHz, CDCl3) δ 7.77 (s, 1H), 7.71 (dt, J=1.6, 7.4, 1H), 7.42-7.38 (m, J=7.4, 1H), 7.38-7.31 (m, 2H), 6.46 (d, J=11.6, 1H), 5.74 (dt, J=7.7, 11.6, 1H), 4.84-4.76 (m, J=7.2, 1H), 3.77 (s, 3H), 2.95 (s, 3H), 2.90 (s, 3H), 2.42-2.30 (m, 4H), 1.83 (p, J=7.2, 2H), 1.64 (s, 2H), 1.54 (d, J=7.2, 3H).

13C NMR (126 MHz, CDCl3) δ 173.78, 172.63, 167.13, 137.88, 134.10, 133.23, 132.09, 128.90, 128.53, 127.12, 125.77, 52.54, 48.67, 37.30, 35.54, 32.60, 28.30, 24.99, 18.31, 17.66.

Other compounds of formula I may be prepared by substituting Ala(OMe) in the above process with other commercially available amino acid esters.

(Z)-(3-(6-(dimethylamino)-6-oxohex-1-en-1-yl)benzoyI)-D-alanine [1] (VSN-44)

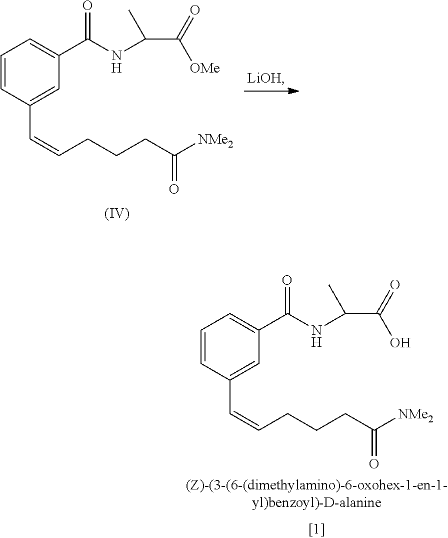 [SMILES: COC(=O)C(C)NC(=O)c1cccc(/C=C\CCCC(=O)N(C)C)c1 ; "(IV)" ; CC(NC(=O)c1cccc(/C=C\CCCC(=O)N(C)C)c1)C(=O)O ; "(Z)-(3-(6-(dimethylamino)-6-oxohex-1-en-1-yl)benzoyl)-D-alanine"]

The ester (IV) (135 mg, 0.41 mmol) in THF (2 mL) was added to lithium hydroxide, hydrate 84 mg, 2 mmol) in water (1 mL). The reaction was stirred at room temperature for 24 hrs. The THF was removed on the rotary evaporator and the residue taken up in 10% aq. Citric acid (10 mL). The aqueous mixture was extracted with DCM (3×30 mL) and dried over Na₂SO₄. Crude yield 307 mg. The product was purified by preparative LCMS (C18) using: Solvent A, 5% MeOH/95% H2O, 0.1% HCOOH. Solvent B, 95% MeOH/5% H₂O, 0.1% HCOOH. Gradient 10% A to 95% over 8 min. The fractions were combined, and the volatiles removed on a rotary evaporator. The final aqueous mixture was freeze dried.

1H NMR (500 MHz, CDCl3) δ 9.03 (s, 1H), 7.74 (s, 1H), 7.73-7.67 (m, J=7.7, 2H), 7.38-7.34 (m, 1H), 7.34-7.31 (m, 1H), 6.43 (d, J=11.6, 1H), 5.70 (dt, J=7.7, 11.6, 1H), 4.80-4.70 (m, 1H), 2.96 (s, 3H), 2.89 (s, 3H), 2.35 (t, J=7.1, 3H), 2.32-2.22 (m, 1H), 1.86-1.73 (m, 2H), 1.54 (d, J=7.2, 3H).

13C NMR (126 MHz, CDCl3) δ 175.39, 173.51, 168.12, 137.75, 133.65, 132.95, 132.31, 128.96, 128.62, 127.07, 126.06, 49.28, 37.55, 35.84, 32.64, 28.27, 25.08, 17.84.

Alternative Synthesis of Intermediate (IIb)

(i) Stage 1

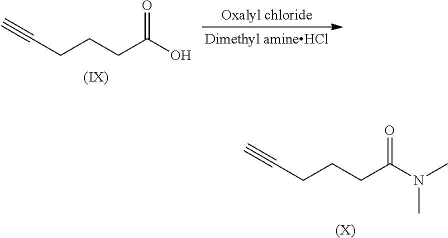

5-Hexynoic acid (553 g, 4.91 mol) and dichloromethane (5.5 L, 10 vol) are charged to a 10 L vessel and cooled to −7° C. Oxalyl chloride (0.475 L, 5.40 mol) is added dropwise maintaining the temperature between −4.5 and 5.0° C. over a 2 h period. The addition apparatus was washed with dichloromethane and stirred for 10 mins at −5° C. Dimethylformamide was added portion-wise with mild effervescence. The temperature was taken to 2° C. and the mixture stirred for 2 h and then warmed to 12° C. and stirred for a further 16 h until no further discernable reaction was observed. The mixture was concentrated to remove all oxalyl chloride. The vessel was rinsed with dichloromethane. Dimethylamine hydrochloride (490 g, 5.89 mol) and dichloromethane (5.5 L, 10 vol) were charged to the 10 L vessel. Triethylamine (2.5 L, 15.70 mol) was charged and the mixture cooled to −10° C. The concentrated acid chloride was treated with dichloromethane (0.3 L, 0.55 vol) and added dropwise maintaining temperature below 6° C. The addition apparatus was rinsed with dichloromethane (50 ml, 0.1 vol) the mixture was stirred at −5° C. for 15 mins and then allowed to warm to ambient temperature. When no further discernable reaction was observed. Water (3 L, 5.5 vol) was charged stirred and the layers partitioned. The aqueous was washed with dichloromethane (2.5 L, 4.5 vol). The organic layers were combined and then washed with 2M Hydrochloric acid (2.5 l, 4.5 vol), 1M NaOH (2.5 L, 4.5 vol), water (3 L, 5.4 vol), brine (2.5 L, 4.5 vol) and dried over MgSO₄ (100 g, 20 wt %). The suspension was filtered and the solvent removed to give a dark oil (X) (214 g, 83%) GF1218-47-128 (568 g, 83%)

(ii) Stage 2

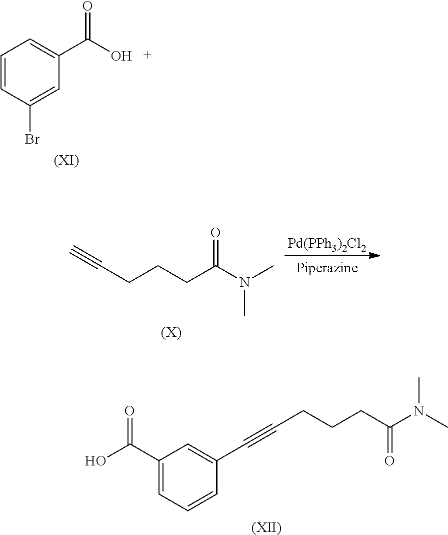

3-Bromobenzoic acid (XI) (631 g, 3.14 mol, 1.0 eq) and piperidine (1.55 L,) were charged to the vessel leading to a mild exotherm and the mixture was heated to 85° C. Dichlorobis(triphenylphosphine)Palladium (II) (44 g, 0.06 mol) was charged, followed by slow addition of N,N-dimethylhex-5-ynamide (X) (656 g, 4.71 mol) maintaining the temperature below 116° C. (reflux). The reaction was stirred for a further 1 hour until no further reaction was observed and allowed to cool to ambient temperature. The resulting viscous mixture was dissolved in water (9 L) an acidified with 5M HCl (4 L) and then extracted with ethyl acetate (5.5,3.5 and 3 L). The organics were combined and washed with water (3 L) and brine (2 L) and then the solvent removed to give a dark oil. The material was taken in acetonitrile (2.5 L) and passed through silica(1.5 Kg) washing with acetonitrile (2.5 L). The resulting solution crystallised and the solid was collected (100 g). The liquors(≈4 L) were concentrated and crystallised to give the desired product as a solid (49 g). The silica was eluted with ethyl acetate (2 L),which yielded further product (54 g) after concentration. A further three portions of ethyl acetate(2 L) were used as eluent to give further product (40 g, 20 g and 17 g) to respectively. The fractions were combined and treated with acetonitrile (340 ml) and recrystallised from the same solvent to give a pale yellow solid (XII) (105 g).

(iii) Stage 3

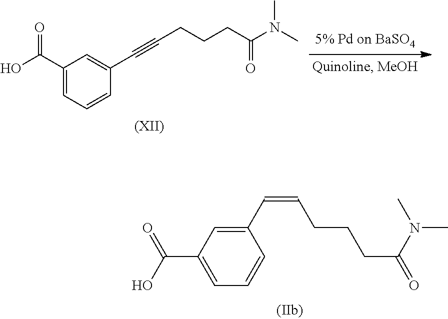

The alkyne (XII) (105 g, 0.4 mol, 1.0 eq) and 5% Pd on BaSO₄ (5.25 g, 5 wt %), methanol (25 vol) and quinoline (3.68 ml, 0.035 vol) were charged to the vessel The vessel was evacuated and the atmosphere replaced with hydrogen three times and then left to react at room temperature under a positive pressure of hydrogen until no further starting material was observable. The solution was degassed and the atmosphere replaced with nitrogen. The suspension was filtered through cellite and washed with methanol (1 L). The solution was then concentrated to dryness and taken in ethyl acetate (5 vol) and washed with 2M HCl (3×2 vol) and brine (3 vol). The solvent was removed and the resulting oil was taken up in acetone (3.3 vol) stirred and cooled until crystallisation occurred. The product was filtered and washed with cold acetone (0.5 vol) to give a colourless solid (IIb) (111 g, 61%).

Synthesis of VSN 45-47

### (R)-methyl 2-(3-iodobenzamido)propanoate

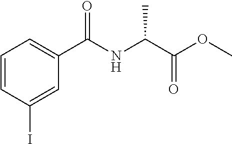 [SMILES: COC(=O)[C@@H](C)NC(=O)c1cccc(I)c1]

Using the general procedure described for amide coupling, the reaction of 3-iodobenzoic acid (22.3 g, 90 mmol), (R)-methyl 2-aminopropanoate.HCl (13.55 g, 97 mmol), HATU (37.6 g, 99 mmol) and TEA (31.3 ml, 225 mmol) in dry DCM (200 mL) gave the title compound (R)-methyl 2-(3-iodobenzamido)propanoate (41 g, 96% yield) as a pale yellow oil. No purification required.

(¹H) DMSO-d₆: 1.40 (3H, d, J=7.3 Hz), 3.64 (3H, s), 4.47 (1H, qn, J=7.3 Hz), 7.30 (1H, t, J=7.8 Hz), 7.89 (1H, ddd, J=1.1, 1.6, 7.8 Hz), 7.93 (1H, ddd, J=1.0, 1.7, 7.8 Hz), 8.25 (1H, t, J=1.6 Hz), 8.92 (1H, d, J=6.8 Hz) ppm. MS(ES+) m/z 334.0 (M+H).

### (R)-6-(3-((1-methoxy-1-oxopropan-2-yl)carbamoyl)phenyl)hex-5-ynoic acid

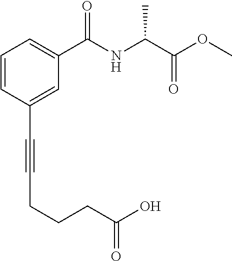 [SMILES: COC(=O)[C@@H](C)NC(=O)c1cccc(C#CCCCC(=O)O)c1]

Following the general method for Sonogashira coupling, the reaction of (R)-methyl 2-(3-iodobenzamido)propanoate (15.0 g, 36.0 mmol) and hex-5-ynoic acid (4.57 ml, 41.4 mmol) after purification by column chromatography (1-3% MeOH in DCM) gave (R)-6-(3-((1-methoxy-1-oxopropan-2-yl)carbamoyl)phenyl)hex-5-ynoic acid (8.43 g, 69.3% yield).

δ(¹H) DMSO-d₆: 1.40 (3H, d, J=7.3 Hz), 1.78 (2H, qn, J=7.2 Hz), 2.39 (2H, t, J=7.3 Hz), 2.46-2.49 (2H, m), 3.64 (3H, s), 4.47 (1H, qn, J=7.3 Hz), 7.46 (1H, t, J=7.8 Hz), 7.56 (1H, td, J=1.3, 7.7 Hz), 7.82 (1H, td, J=1.4, 7.8 Hz), 7.92 (1H, t, J=1.5 Hz), 8.87 (1H, d, J=6.9 Hz), 12.16 (1H, s) ppm. MS(ES+) m/z 318 (M+H).

### (R)-methyl 2-(3-(6-oxo-6-(pyrrolidin-1-yl)hex-1-yn-1-yl)benzamido)propanoate VSN 45

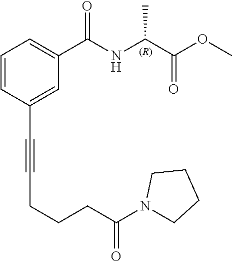

Using the general procedure described for amide coupling, the reaction of (R)-6-(3-((1-methoxy-1-oxopropan-2-yl)carbamoyl)phenyl)hex-5-ynoic acid (0.80 g, 2.52 mmol), pyrrolidine (0.22 ml, 2.65 mmol), DIPEA (1.35 ml, 7.56 mmol), HATU (1.15 g, 3.03 mmol) and dry DCM (10 mL) after purfication by chromatography (1-4% MeOH in DCM) gave the title compound (R)-methyl 2-(3-(6-oxo-6-(pyrrolidin-1-yl)hex-1-yn-1-yl)benzamido)propanoate (0.6 g, 63.0% yield) as a pale yellow oil.

δ(¹H) DMSO-d₆: 1.40 (3H, d, J=7.3 Hz), 1.69-1.93 (6H, m), 2.39 (2H, t, J=7.2 Hz), 2.44-2.52 (2H, m), 3.28 (2H, t, J=6.8 Hz), 3.41 (2H, t, J=6.8 Hz), 3.64 (3H, s), 4.47 (1H, qn, J=7.3 Hz), 7.46 (1H, t, J=7.7 Hz), 7.56 (1H, td, J=1.3, 7.7 Hz), 7.82 (1H, td, J=1.5, 7.8 Hz), 7.92 (1H, t, J=1.5 Hz), 8.87 (1H, d, J=6.9 Hz) ppm.

δ(¹³C) DMSO-d₆: 16.65, 18.23, 32.56, 38.22, 45.21, 45.84, 48.28, 51.89, 80.20, 91.12, 123.20, 127.08, 128.74, 130.03, 133.91, 133.98, 165.41, 169.67, 173.05 ppm. MS(ES+) m/z 371 (M+H).

### (R,Z)-methyl 2-(3-(6-oxo-6-(pyrrolidin-1-yl)hex-1-en-1-yl)benzamido)propanoate VSN 46

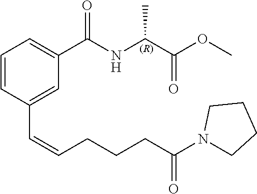

Following the general procedure for Lindlar reduction, the hydrogenation of (R)-methyl 2-(3-(6-oxo-6-(pyrrolidin-1-yl)hex-1-yn-1-yl)benzamido)propanoate (0.5 g, 1.350 mmol) gave the named product with trace amounts of the trans double bond isomer and fully saturated products (determined by ¹H NMR). Separation by column chromatography (1-3% MeOH in DCM) gave the title compound (0.17 g, 33.1%). The other 2 components were not isolated.

δ(¹H) DMSO-d₆: 1.40 (3H, d, J=7.3 Hz), 1.60-1.76 (4H, m), 1.82 (2H, qn, J=6.6 Hz), 2.25 (2H, t, J=7.2 Hz), 2.31 (2H, dq, J=1.6, 7.5 Hz), 3.22 (2H, t, J=6.9 Hz), 3.31-3.36 (2H, m), 3.64 (3H, s), 4.48 (1H, qn, J=7.3 Hz), 5.74 (1H, td, J=7.3, 11.7 Hz), 6.48 (1H, d, J=11.7 Hz), 7.42-7.49 (2H, m), 7.70-7.80 (2H, m), 8.81 (1H, d, J=6.9 Hz) ppm.

δ(¹³C) DMSO-d₆: 16.70, 23.89, 24.44, 25.55, 27.66, 33.03, 45.15, 45.79, 48.25, 51.86, 125.69, 127.47, 128.25, 128.36, 131.35, 133.30, 133.74, 137.11, 166.14, 169.98, 173.12 ppm.

MS(ES+) m/z 373 (M+H).

### (R,Z)-2-(3-(6-oxo-6-(pyrrolidin-1-yl)hex-1-en-1-yl)benzamido)propanoic acid VSN 47

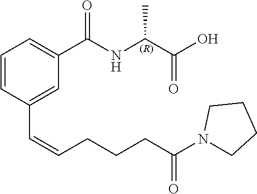

Following the general procedure for saponification, the reaction of (R,Z)-methyl 2-(3-(6-oxo-6-(pyrrolidin-1-yl)hex-1-en-1-yl)benzamido)propanoate (0.15 g, 0.40 mmol) with lithium hydroxide (19 mg, 0.81 mmol) gave (R,Z)-2-(3-(6-oxo-6-(pyrrolidin-1-yl)hex-1-en-1-yl)benzamido)propanoic acid (0.13 g, 88% yield) as a white solid.

δ(¹H) DMSO-d₆: 1.39 (3H, d, J=7.4 Hz), 1.68 (4H, m), 1.81 (2H, qn, J=6.6 Hz), 2.24 (2H, t, J=7.2 Hz), 2.27-2.37 (2H, m), 3.22 (2H, t, J=6.8 Hz), 3.32 (2H, t, J=6.8 Hz), 24.42 (1H, qn, J=7.3 Hz), 5.74 (1H, td, J=7.3, 11.7 Hz), 6.48 (1H, d, J=11.7 Hz), 7.41-7.50 (2H, m), 7.71-7.81 (2H, m), 8.71 (1H, d, J=7.3 Hz), 12.54 (1H, s) ppm.

δ(¹³C) DMSO-d₆: 16.88, 23.94, 24.49, 25.59, 27.70, 33.07, 45.20, 45.83, 48.17, 125.71, 127.52, 128.25, 128.43, 131.28, 133.30, 134.00, 137.10, 166.06, 170.02, 174.23 ppm.

MS(ES+) m/z 359 (M+H).

Synthesis of VSN 48-50

### 6-(3-(methoxycarbonyl)phenyl)hex-5-ynoic acid

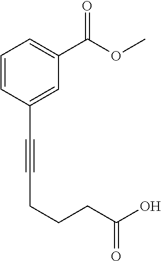 [SMILES: COC(=O)c1cccc(C#CCCCC(=O)O)c1]

Following the general method for Sonogashira coupling, the reaction of methyl 3-iodobenzoate (1.0 g, 3.82 mmol) and hex-5-ynoic acid (0.421 ml, 3.82 mmol) after purification by column chromatography (0-3% MeOH in DCM) gave 6-(3-(methoxycarbonyl)phenyl)hex-5-ynoic acid (0.78 g, 81% yield).

δ(¹H) DMSO-d₆: 1.79 (2H, qn, J=7.2 Hz), 2.39 (2H, t, J=7.3 Hz), 2.45-2.49 (2H, m), 3.86 (3H, s), 7.47-7.55 (1H, m), 7.66 (1H, td, J=1.4, 7.7 Hz), 7.88-7.94 (2H, m), 12.07 (1H, s) ppm.

MS(ES+) m/z 247 (M+H).

### Methyl 3-(6-(dimethylamino)-6-oxohex-1-yn-1-yl)benzoate

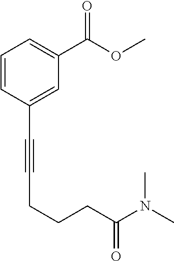 [SMILES: COC(=O)c1cccc(C#CCCCC(=O)N(C)C)c1]

Following the general procedure described for amide coupling, the reaction 6-(3-(methoxycarbonyl)phenyl)hex-5-ynoic acid (5.2 g, 21.12 mmol), dimethylamine.HCl (2.07 g, 25.3 mmol), DIPEA (11.28 ml, 63.3 mmol) and HATU (10.4 g, 27.5 mmol) in dry DCM (50 mL) gave methyl 3-(6-(dimethylamino)-6-oxohex-1-yn-1-yl)benzoate (5.3 g, 87% yield) as an orange oil. No purification required.

δ(¹H) DMSO-d₆: 1.77 (2H, qn, J=7.2 Hz), 2.36-2.49 (4H, m), 2.82 (3H, s), 2.97 (3H, s), 3.86 (3H, s), 7.47-7.54 (1H, m), 7.66 (1H, td, J=1.4, 7.7 Hz), 7.89-7.91 (2H, m) ppm.

MS(ES+) m/z 274 (M+H).

### 3-(6-(dimethylamino)-6-oxohex-1-yn-1-yl)benzoic acid

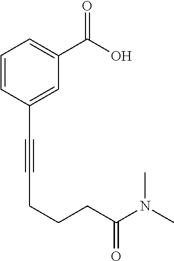 [SMILES: CN(C)C(=O)CCCC#Cc1cccc(C(=O)O)c1]

To a solution of methyl 3-(6-(dimethylamino)-6-oxohex-1-yn-1-yl)benzoate (5.30 g, 18.42 mmol) in THF (40 mL) and water (20 mL) was added lithium hydroxide (0.88 g, 36.8 mmol). The reaction was stirred at rt until judged complete by HPLC analysis. The volatiles were then removed in vacuo and the residue was partitioned between water (60 mL) and EtOAc (50 mL). The aqueous layer was then acidified to pH 1 with 1 N HCl (aq) and extracted with EtOAc (3×100 mL). Next, the combined organic extracts were washed with water (2×75 mL) and brine (50 mL) then dried (Na₂SO₄), filtered and concentrated in vacuo to a residue. This was azeotroped with iso-hexanes and dried in vacuum desiccator (45° C.) to give 3-(6-(dimethylamino)-6-oxohex-1-yn-1-yl)benzoic acid (4.3 g, 88% yield) as an orange solid.

δ(¹H) DMSO-d₆: 1.78 (2H, qn, J=7.2 Hz), 2.41-2.49 (4H, m), 2.83 (3H, s), 2.98 (3H, s), 7.46-7.52 (1H, m), 7.64 (1H, td, J=1.5, 7.7 Hz), 7.87-7.91 (2H, m), 13.11 (1H, s) ppm.

MS(ES+) m/z 260 (M+H).

### methyl 2-(3-(6-(dimethylamino)-6-oxohex-1-yn-1-yl)benzamido)acetate VSN 48

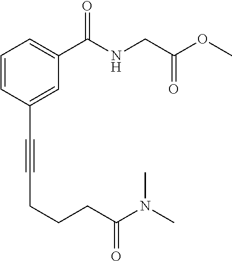 [SMILES: COC(=O)CNC(=O)c1cccc(C#CCCCC(=O)N(C)C)c1]

Using the general procedure described for amide coupling, the reaction of 3-(6-(dimethylamino)-6-oxohex-1-yn-1-yl)benzoic acid (0.70 g, 2.65 mmol), methyl 2-aminoacetate.HCl (0.38 g, 3.04 mmol), DIPEA (1.4 ml, 7.94 mmol) and HATU (1.31 g, 3.44 mmol) in dry DCM (10 mL) after purification by chromatography (1-3% MeOH in DCM) gave the title compound methyl 2-(3-(6-(dimethylamino)-6-oxohex-1-yn-1-yl)benzamido)acetate (0.86 g, 93% yield) as a pale yellow oil.

δ(¹H) DMSO-d₆: 1.77 (2H, qn, J=7.2 Hz), 2.42-2.48 (4H, m), 2.82 (3H, s), 2.97 (3H, s), 3.65 (3H, s), 4.01 (2H, d, J=5.8 Hz), 7.47 (1H, t, J=7.8 Hz), 7.57 (1H, td, J=1.3, 7.7 Hz), 7.81 (1H, td, J=1.4, 7.8 Hz), 7.89 (1H, t, J=1.5 Hz), 9.04 (1H, t, J=5.8 Hz) ppm.

δ(¹³C) DMSO-d₆: 18.22, 23.92, 31.18, 34.77, 36.63, 38.22, 41.19, 51.75, 80.14, 91.18, 123.33, 126.85, 128.85, 129.92, 133.86 and 134.05, 165.81, 170.25, 171.30 ppm.

MS(ES+) m/z 331 (M+H).

### (Z)-methyl 2-(3-(6-(dimethylamino)-6-oxohex-1-en-1-yl)benzamido)acetate VSN 49

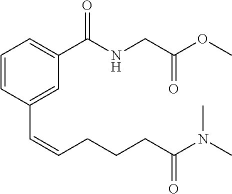 [SMILES: COC(=O)CNC(=O)c1cccc(/C=C\CCCC(=O)N(C)C)c1]

Following the general procedure for the Lindlar reduction, the hydrogenation of methyl 2-(3-(6-(dimethylamino)-6-oxohex-1-yn-1-yl)benzamido)acetate (0.40 g, 1.21 mmol) gave the named product with trace amounts of the trans double bond isomer and fully saturated products (determined by ¹H NMR). Separation by column chromatography (1-3% MeOH in DCM) gave the title compound (0.21 g, 51.1% yield). The other 2 components were not isolated.

δ(¹H) DMSO-d₆: 1.65 (2H, qn, J=7.3 Hz), 2.27-2.36 (4H, m), 2.77 (3H, s), 2.92 (3H, s), 3.66 (3H, s), 4.02 (2H, d, J=5.9 Hz), 5.75 (1H, dt, J=7.4, 11.7 Hz), 6.47 (1H, br d, J=11.7 Hz), 7.47 (2H, dd, J=1.2, 4.0 Hz), 7.70-7.81 (2H, m), 8.98 (1H, t, J=5.8 Hz) ppm.

δ(¹³C) DMSO-d₆: 24.73, 27.73, 31.69, 34.74, 36.62, 41.23, 51.72, 125.53, 127.21, 128.29, 128.40, 131.50, 133.34, 133.65, 137.20, 166.50, 170.35, 171.65 ppm.

MS(ES+) m/z 333 (M+H).

(Z)-2-(3-(6-(dimethylamino)-6-oxohex-1-en-1-yl)benzamido)acetic acid VSN 50

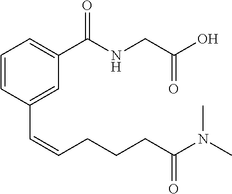 [SMILES: CN(C)C(=O)CCC/C=C\c1cccc(C(=O)NCC(=O)O)c1]

Following the general procedure for saponification, the reaction of (Z)-methyl 2-(3-(6-(dimethylamino)-6-oxohex-1-en-1-yl)benzamido)acetate (0.1 g, 0.30 mmol) with lithium hydroxide (14 mg, 0.60 mmol) gave (Z)-2-(3-(6-(dimethylamino)-6-oxohex-1-en-1-acid (76 mg, 77% yield) as a colourless gum.

δ(¹H) DMSO-d₆: 1.66 (2H, qn, J=7.3 Hz), 2.25-2.38 (4H, m), 2.78 (3H, s), 2.92 (3H, s), 3.94 (2H, d, J=5.9 Hz), 5.75 (1H, dt, J=7.4, 11.7 Hz), 6.48 (1H, d, J=11.8 Hz), 7.47 (2H, dd, J=1.5, 3.9 Hz), 7.71-7.77 (1H, m), 7.79 (1H, s), 8.87 (1H, t, J=5.8 Hz), 12.56 (1H, s) ppm.

δ(¹³C) DMSO-d₆: 24.74, 27.74, 31.71, 34.75, 36.63, 41.23, 125.52, 127.21, 128.33, 128.36, 131.39, 133.29, 133.88, 137.16, 166.37, 171.29, 171.66 ppm.

MS(ES+) m/z 319 (M+H).

Synthesis of VSN 51 to 53

### (S)-methyl 2-(3-(6-(dimethylamino)-6-oxohex-1-yn-1-yl)benzamido)-3-hydroxypropanoate VSN 51

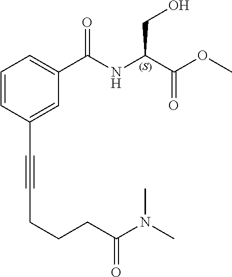

Using the general procedure described for amide coupling, the reaction of 3-(6-(dimethylamino)-6-oxohex-1-yn-1-yl)benzoic acid (0.70 g, 2.65 mmol), (S)-methyl 2-amino-3-hydroxypropanoate.HCl (0.45 g, 2.91 mmol), DIPEA (1.18 ml, 6.61 mmol) and HATU (1.16 g, 3.04 mmol) in dry DCM (10 mL) after purfication by chromatography (1-5% MeOH in DCM) gave the title compound (S)-methyl 2-(3-(6-(dimethylamino)-6-oxohex-1-yn-1-yl)benzamido)-3-hydroxypropanoate (0.67 g, 68.9% yield) as a pale yellow oil.

δ(¹H) DMSO-d₆: 1.78 (2H, qn, J=7.2 Hz), 2.42-2.49 (4H, m), 2.82 (3H, s), 2.97 (3H, s), 3.65 (3H, s), 3.79 (2H, t, J=5.9 Hz), 4.52 (1H, dt, J=5.5, 7.4 Hz), 5.04 (1H, t, J=6.2 Hz), 7.47 (1H, t, J=7.7 Hz), 7.57 (1H, td, J=1.3, 7.7 Hz), 7.83 (1H, td, J=1.5, 7.8 Hz), 7.93 (1H, t, J=1.5 Hz), 8.68 (1H, d, J=7.4 Hz) ppm.

δ(¹³C) DMSO-d₆: 18.23, 23.93, 31.20, 34.78, 36.63, 51.89, 55.71, 60.94, 80.18, 91.16, 123.21, 127.12, 128.76, 130.07, 133.98, 134.04, 165.73, 170.92, 171.30 ppm.

MS(ES+) m/z 361 (M+H).

### (S,Z)-methyl 2-(3-(6-(dimethylamino)-6-oxohex-1-en-1-yl)benzamido)-3-hydroxypropanoate VSN 52

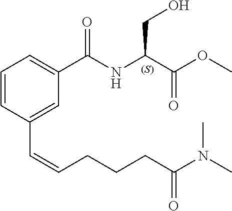

Following the general procedure for the Lindlar reduction, the hydrogenation of (S)-methyl 2-(3-(6-(dimethylamino)-6-oxohex-1-yn-1-yl)benzamido)-3-hydroxypropanoate (0.50 g, 1.39 mmol) gave the named product along with the trans double bond isomer (10%) and fully saturated product (20%) (determined by ¹H NMR). Separation by column chromatography (1-3% MeOH in DCM) gave the title compound (0.28 g, 54.6% yield). The other 2 components were not isolated.

δ(¹H) DMSO-d₆: 1.65 (2H, qn, J=7.4 Hz), 2.26-2.35 (4H, m), 2.77 (3H, s), 2.91 (3H, s), 3.65 (3H, s), 3.79 (2H, t, J=5.8 Hz), 4.54 (1H, dt, J=5.4, 7.4 Hz), 5.04 (1H, t, J=6.2 Hz), 5.75 (1H, dt, J=7.3, 11.7 Hz), 6.48 (1H, d, J=11.7 Hz), 7.44-7.50 (2H, m), 7.71-7.81 (2H, m), 8.60 (1H, d, J=7.4 Hz) ppm.

δ(¹³C) DMSO-d₆: 24.79, 27.71, 31.72, 34.74, 36.62, 51.86, 55.66, 60.99, 125.67, 127.57, 128.29, 128.33, 131.36, 133.33, 133.81, 137.15, 166.45, 171.03, 171.61 ppm.

MS(ES+) m/z 363 (M+H).

### (S,Z)-2-(3-(6-(dimethylamino)-6-oxohex-1-en-1-yl)benzamido)-3-hydroxypropanoic acid VSN 53

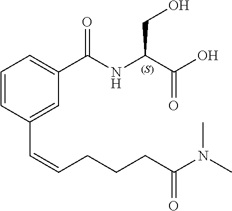

Following the general procedure for saponification, the reaction of (S,Z)-methyl 2-(3-(6-(dimethylamino)-6-oxohex-1-en-1-yl)benzamido)-3-hydroxypropanoate (0.15 g, 0.42 mmol) with lithium hydroxide (20 mg, 0.83 mmol) gave (S,Z)-2-(3-(6-(dimethylamino)-6-oxohex-1-en-1-yl)benzamido)-3-hydroxypropanoic acid (75 mg, 51.0% yield) as a colourless gum.

δ(¹H) DMSO-d₆: 1.65 (2H, qn, J=7.4 Hz), 2.18-2.41 (4H, m), 2.77 (3H, s), 2.91 (3H, s), 3.80 (2H, d, J=5.2 Hz), 4.48 (1H, dt, J=7.7, 5.2 Hz), 4.95 (1H, br s), 5.75 (1H, dt, J=7.3, 11.7 Hz), 6.49 (1H, d, J=11.8 Hz), 7.47 (2H, d, J=5.0 Hz), 7.66-7.86 (2H, m), 8.42 (1H, d, J=7.7 Hz), 12.67 (1H, s) ppm.

δ(¹³C) DMSO-d₆: 24.80, 27.72, 31.73, 34.75, 36.63, 55.66, 61.16, 125.61, 127.53, 128.26, 128.37, 131.25, 133.29, 134.07, 137.13, 166.32, 171.62, 171.90 ppm.

MS(ES+) m/z 349 (M+H).

Synthesis of VSN 54 to 56

### (S)-methyl 2-(3-(6-(dimethylamino)-6-oxohex-1-yn-1-yl)benzamido)-3-phenylpropanoate VSN 54

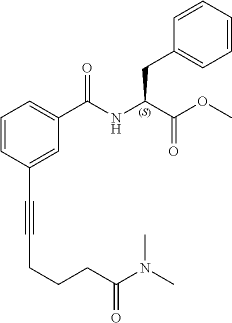

Using the general procedure described for amide coupling, the reaction of 3-(6-(dimethylamino)-6-oxohex-1-yn-1-yl)benzoic acid (0.70 g, 2.65 mmol), (S)-methyl 2-amino-3-phenylpropanoate.HCl (0.60 g, 2.78 mmol), DIPEA (1.18 ml, 6.61 mmol) and HATU (1.16 g, 3.04 mmol) in dry DCM (10 mL) after purfication by chromatography (1-4% MeOH in DCM) gave the title compound (S)-methyl 2-(3-(6-(dimethylamino)-6-oxohex-1-yn-1-yl)benzamido)-3-phenylpropanoate (0.83 g, 72.4% yield) as a pale yellow oil.

δ(¹H) DMSO-d₆: 1.71-1.84 (2H, m), 2.42-2.48 (41-I, m), 2.82 (3H, s), 2.97 (3H, s), 3.08 (1H, dd, J=10.1, 13.8 Hz), 3.17 (1H, dd, J=5.3, 13.8 Hz), 3.64 (3H, s), 4.66 (1H, ddd, J=5.3, 7.8, 10.1 Hz), 7.16-7.23 (1H, m), 7.24-7.32 (4H, m), 7.43 (1H, t, J=7.7 Hz), 7.54 (1H, td, J=1.3, 7.7 Hz), 7.73 (1H, td, J=1.4, 7.8 Hz), 7.83 (1H, t, J=1.5 Hz), 8.93 (1H, d, J=7.8 Hz) ppm.

δ(¹³C) DMSO-d₆: 18.22, 23.91, 31.18, 34.78, 36.14, 36.62, 51.97, 54.25, 80.16, 91.17, 123.21, 126.48, 127.01, 128.23, 128.76, 129.00, 129.94, 133.87, 134.05, 137.64, 165.60, 171.30, 172.05 ppm.

MS(ES+) m/z 421 (M+H).

### (S,Z)-methyl 2-(3-(6-(dimethylamino)-6-oxohex-1-en-1-yl)benzamido)-3-phenylpropanoate VSN 55

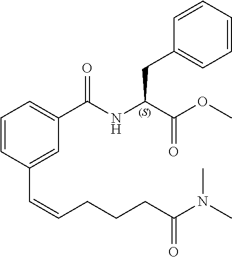

Following the general procedure for the Lindlar reduction, the hydrogenation of (S)-methyl 2-(3-(6-(dimethylamino)-6-oxohex-1-yn-1-yl)benzamido)-3-phenylpropanoate (0.5 g, 1.19 mmol) gave the named product along with the trans double bond isomer (5%) and fully saturated product (10%) (determined by ¹H NMR). Separation by column chromatography (1-2% MeOH in DCM) gave the title compound (0.33 g, 64.4% yield). The other 2 components were not isolated.

δ(¹H) DMSO-d₆: 1.64 (2H, qn, J=7.4 Hz), 2.23-2.36 (4H, m), 2.77 (3H, s), 2.90 (3H, s), 3.09 (1H, dd, J=10.3, 13.7 Hz), 3.17 (1H, dd, J=5.1, 13.7 Hz), 3.64 (3H, s), 4.66 (1H, ddd, J=5.2, 7.9, 10.2 Hz), 5.74 (1H, dt, J=7.3, 11.8 Hz), 6.45 (1H, br d, J=11.7 Hz), 7.13-7.23 (1H, m), 7.24-7.33 (4H, m), 7.40-7.47 (2H, m), 7.61-7.69 (2H, m), 8.87 (1H, d, J=7.9 Hz) ppm.

δ(¹³C) DMSO-d₆: 24.76, 27.68, 31.70, 34.73, 36.17, 36.60, 51.94, 54.27, 125.64, 126.46, 127.35, 128.21, 128.28, 129.05, 131.44, 133.33, 133.75, 137.09, 137.71, 166.40, 171.59, 172.15 ppm.

MS(ES+) m/z 423 (M+H).

### (S,Z)-2-(3-(6-(dimethylamino)-6-oxohex-1-en-1-yl)benzamido)-3-phenylpropanoic acid VSN 56

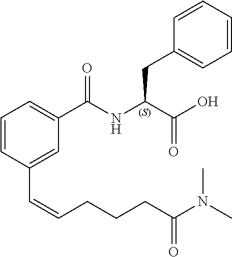

Following the general procedure for saponification, the reaction of (S,Z)-methyl 2-(3-(6-(dimethylamino)-6-oxohex-1-en-1-yl)benzamido)-3-phenylpropanoate (0.20 g, 0.47 mmol) with lithium hydroxide (23 mg, 0.95 mmol) gave (S,Z)-2-(3-(6-(dimethylamino)-6-oxohex-1-en-1-yl)benzamido)-3-phenylpropanoic acid (131 mg, 66.4% yield) as a colourless gum.

δ(¹H) DMSO-d₆: 1.64 (2H, qn, J=7.4 Hz), 2.24-2.35 (4H, m), 2.77 (3H, s), 2.90 (3H, s), 3.06 (1H, dd, J=10.8, 13.7 Hz), 3.19 (1H, dd, J=4.3, 13.7 Hz), 5.73 (1H, td, J=7.3, 11.7 Hz), 6.45 (1H, d, J=11.8 Hz), 7.14-7.21 (2H, m), 7.24-7.28 (2H,m), 7.29-7.35 (2H, m), 7.42 (2H, d, J=5.3 Hz), 7.61-7.70 (2H, m), 8.72 (1H, d, J=8.2 Hz), 12.76 (1H, s) ppm.

δ(¹³C) DMSO-d₆: 24.78, 27.69, 31.71, 34.75, 36.21, 36.61, 54.20, 125.30, 126.32, 127.33, 128.15, 128.24, 128.31, 129.03, 131.31, 133.28, 134.01, 137.05, 138.19, 166.32, 171.60, 173.14 ppm.

MS(ES+) m/z 409 (M+H).

Synthesis of VSN 57 to 59

### (R)-methyl 2-(3-(6-(dimethylamino)-6-oxohex-1-yn-1-yl)benzamido)-3-hydroxypropanoate VSN 57

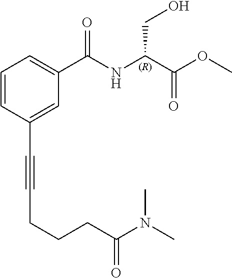

Using the general procedure described for amide coupling, the reaction of 3-(6-(dimethylamino)-6-oxohex-1-yn-1-yl)benzoic acid (0.70 g, 2.65 mmol), (R)-methyl 2-amino-3-hydroxypropanoate.HCl (0.45 g, 2.91 mmol), DIPEA (1.4 ml, 7.94 mmol) and HATU (1.21 g, 3.17 mmol) in dry DCM (10 mL) after purfication by chromatography (1-5% MeOH in DCM) gave the title compound (R)-methyl 2-(3-(6-(dimethylamino)-6-oxohex-1-yn-1-yl)benzamido)-3-hydroxypropanoate (0.77 g, 79% yield) as a viscous pale yellow oil.

δ(¹H) DMSO-d₆: 1.78 (2H, qn, J=7.2 Hz), 2.42-2.49 (4H, m), 2.82 (3H, s), 2.97 (3H, s), 3.65 (3H, s), 3.79 (2H, t, J=5.9 Hz), 4.52 (1H, dt, J=5.5, 7.4 Hz), 5.04 (1H, t, J=6.2 Hz), 7.47 (1H, t, J=7.7 Hz), 7.57 (1H, td, J=1.3, 7.7 Hz), 7.83 (1H, td, J=1.5, 7.8 Hz), 7.93 (1H, t, J=1.5 Hz), 8.68 (1H, d, J=7.4 Hz) ppm.

δ(¹³C) DMSO-d₆: 18.24, 23.93, 31.20, 34.78, 36.63, 51.88, 55.71, 60.94, 80.18, 91.16, 123.21, 127.12, 128.76, 130.07, 133.99, 134.04, 165.73, 170.92, 171.30 ppm.

MS(ES+) m/z 361 (M+H).

### (R,Z)-methyl 2-(3-(6-(dimethylamino)-6-oxohex-1-en-1-yl)benzamido)-3-hydroxypropanoate VSN 58

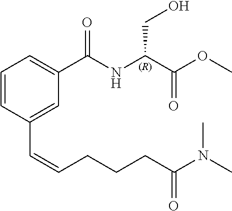

Following the general procedure for the Lindlar reduction, the hydrogenation of (R)-methyl 2-(3-(6-(dimethylamino)-6-oxohex-1-yn-1-yl)benzamido)-3-hydroxypropanoate (0.50 g, 1.39 mmol) gave the named product along with the trans double bond isomer (5%) and fully saturated product (5%) (determined by ¹H NMR). Separation by column chromatography (1-3% MeOH in DCM) gave the title compound (0.32 g, 62.4% yield). The other 2 components were not isolated.

δ(¹H) DMSO-d₆: 1.66 (2H, qn, J=7.4 Hz), 2.20-2.39 (4H, m), 2.78 (3H, s), 2.92 (3H, s), 3.66 (3H, s), 3.80 (2H, t, J=5.8 Hz), 4.54 (1H, dt, J=5.4, 7.4 Hz), 5.05 (1H, t, J=6.2 Hz), 5.76 (1H, dt, J=7.3, 11.7 Hz), 6.49 (1H, br d, J=11.7 Hz), 7.42-7.53 (2H, m), 7.71-7.82 (2H, m), 8.60 (1H, d, J=7.5 Hz) ppm.

δ(¹³C) DMSO-d₆: 24.78, 27.71, 31.72, 34.74, 36.62, 51.86, 55.66, 60.99, 125.66, 127.56, 128.28, 128.33, 131.36, 133.33, 133.81, 137.15, 166.44, 171.02, 171.61 ppm.

MS(ES+) m/z 363 (M+H).

### (R,Z)-2-(3-(6-(dimethylamino)-6-oxohex-1-en-1-yl)benzamido)-3-hydroxypropanoic acid VSN 59

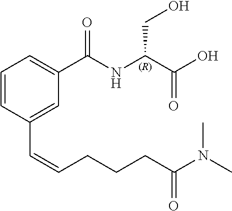

Following the general procedure for saponification, the reaction of (R,Z)-methyl 2-(3-(6-(dimethylamino)-6-oxohex-1-en-1-yl)benzamido)-3-hydroxypropanoate (0.15 g, 0.41 mmol) with lithium hydroxide (25 mg, 1.04 mmol) gave (R,Z)-2-(3-(6-(dimethylamino)-6-oxohex-1-en-1-yl)benzamido)-3-hydroxypropanoic acid (77 mg, 52.3% yield) as a colourless gum.

δ(¹H) DMSO-d₆: 1.65 (2H, qn, J=7.4 Hz), 2.25-2.38 (4H, m), 2.77 (3H, s), 2.91 (3H, s), 3.79 (2H, d, J=5.2 Hz), 4.47 (1H, dt, J=5.2, 7.6 Hz), 5.60-5.89 (1H, m), 6.49 (1H, d, J=11.7 Hz), 7.34-7.59 (3H, m), 7.64-7.88 (2H, m), 8.41 (1H, d, J=7.7 Hz), 12.58 (1H, s) ppm.

δ(¹³C) DMSO-d₆: 24.79, 27.72, 31.73, 34.75, 36.63, 55.65, 61.16, 125.61, 127.53, 128.26, 128.37, 131.25, 133.29, 134.07, 137.13, 166.32, 171.61, 171.90 ppm.

MS(ES+) m/z 349 (M+H).

Synthesis of VSN 60 to 62

### (R)-methyl 2-(3-(6-(dimethylamino)-6-oxohex-1-yn-1-yl)benzamido)-3-phenylpropanoate VSN 60

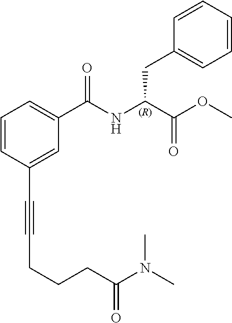

Using the general procedure described for amide coupling, the reaction of 3-(6-(dimethylamino)-6-oxohex-1-yn-1-yl)benzoic acid (0.70 g, 2.65 mmol), (R)-methyl 2-amino-3-phenylpropanoate.HCl (0.6 g, 2.78 mmol), DIPEA (1.4 ml, 7.94 mmol) and HATU (1.3 g, 3.44 mmol) in dry DCM (10 mL) after purfication by chromatography (1-4% MeOH in DCM) gave the title compound (R)-methyl 2-(3-(6-(dimethylamino)-6-oxohex-1-yn-1-yl)benzamido)-3-phenylpropanoate (0.81 g, 68.4% yield) as a viscous pale yellow oil.

δ(¹H) DMSO-d₆: 1.74-1.82 (2H, m), 2.42-2.48 (4H, m), 2.82 (3H, s), 2.97 (3H, s), 3.08 (1H, dd, J=10.1, 13.8 Hz), 3.17 (1H, dd, J=5.3, 13.8 Hz), 3.64 (3H, s), 4.66 (1H, ddd, J=5.3, 7.8, 10.1 Hz), 7.16-7.23 (1H, m), 7.24-7.32 (4H, m), 7.43 (1H, t, J=7.7 Hz), 7.54 (1H, td, J=1.3, 7.7 Hz), 7.73 (1H, td, J=1.4, 7.8 Hz), 7.83 (1H, t, J=1.5 Hz), 8.93 (1H, d, J=7.8 Hz) ppm.

δ(¹³C) DMSO-d₆: 18.22, 23.91, 31.18, 34.78, 36.14, 36.62, 51.96, 54.25, 80.16, 91.17, 123.21, 126.48, 127.01, 128.22, 128.75, 129.00, 129.94, 133.87, 134.04, 137.63, 165.60, 171.29, 172.05 ppm.

MS(ES+) m/z 421 (M+H).

### (R,Z)-methyl 2-(3-(6-(dimethylamino)-6-oxohex-1-en-1-yl)benzamido)-3-phenylpropanoate VSN 61

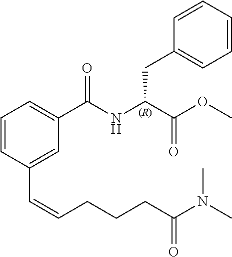

Following the general procedure for the Lindlar reduction, the hydrogenation of (R)-methyl 2-(3-(6-(dimethylamino)-6-oxohex-1-yn-1-yl)benzamido)-3-phenylpropanoate (0.50 g, 1.19 mmol) gave the named product along with the trans double bond isomer (5%) and fully saturated product (10%) (determined by ¹H NMR). Separation by column chromatography (1-2% MeOH in DCM) gave the title compound (0.37 g, 72.2% yield). The other 2 components were not isolated.

δ(¹H) DMSO-d₆: 1.65 (2H, qn, J=7.4 Hz), 2.25-2.38 (4H, m), 2.78 (3H, s), 2.91 (3H, s), 3.10 (1H, dd, J=10.3, 13.7 Hz), 3.18 (1H, dd, J=5.1, 13.7 Hz), 3.65 (3H, s), 4.67 (1H, ddd, J=5.2, 7.9, 10.2 Hz), 5.75 (1H, dt, J=7.4, 11.8 Hz), 6.46 (1H, br d, J=11.6 Hz), 7.15-7.24 (1H, m), 7.24-7.36 (4H, m), 7.44 (2H, d, J=5.0 Hz), 7.61-7.71 (2H, m), 8.87 (1H, d, J=8.0 Hz) ppm.

δ(¹³C) DMSO-d₆: 24.76, 27.68, 31.70, 34.73, 36.17, 36.60, 51.94, 54.27, 125.64, 126.46, 127.35, 128.21, 128.28, 129.04, 131.43, 133.33, 133.75, 137.09, 137.71, 166.40, 171.59, 172.15 ppm.

MS(ES+) m/z 423 (M+H).

### (R,Z)-2-(3-(6-(dimethylamino)-6-oxohex-1-en-1-yl)benzamido)-3-phenylpropanoic acid VSN 62

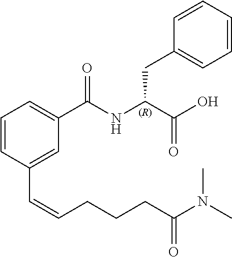

Following the general procedure for saponification, the reaction of (R,Z)-methyl 2-(3-(6-(dimethylamino)-6-oxohex-1-en-1-yl)benzamido)-3-phenylpropanoate (0.20 g, 0.47 mmol) with lithium hydroxide (23 mg, 0.95 mmol) gave (R,Z)-2-(3-(6-(dimethylamino)-6-oxohex-1-en-1-yl)benzamido)-3-phenylpropanoic acid (0.18 g, 0.43 mmol, 91% yield) as a colourless gum.

δ(¹H) DMSO-d₈: 1.66 (2H, on, J=7.4 Hz), 2.24-2.35 (4H, m), 2.77 (3H, s), 2.91 (3H, s), 3.08 (1H, dd, J=10.5, 13.8 Hz), 3.16-3.22 (1H, m), 4.64 (1H, td, J=4.5, 10.2 Hz), 5.74 (1H, dt, J=7.3, 11.7 Hz), 6.45 (1H, d, J=11.7 Hz), 7.18 (1H, t, J=7.1 Hz), 7.22-7.34 (4H, m), 7.42 (2H, d, J=5.1 Hz), 7.59-7.71 (2H, m), 8.62 (1H, br d, J=4.4 Hz), 12.63 (1H, s) ppm.

δ(¹³C) DMSO-d₆: 24.78, 27.69, 31.71, 34.75, 36.21, 36.61, 54.21, 125.60, 126.32, 127.33, 128.15, 128.24, 128.32, 129.04, 131.31, 133.28, 134.01, 137.05, 138.20, 166.31, 171.60, 173.15 ppm.

MS(ES+) m/z 409 (M+H).

Synthesis of VSN 63 to 65

### (S)-methyl 2-(3-(6-(dimethylamino)-6-oxohex-1-yn-1-yl)benzamido)propanoate VSN 63

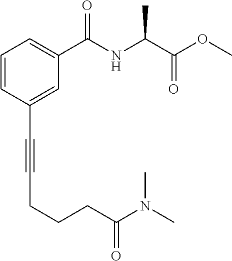 [SMILES: COC(=O)[C@H](C)NC(=O)c1cccc(C#CCCCC(=O)N(C)C)c1]

To a solution of 3-(6-(dimethylamino)-6-oxohex-1-yn-1-yl)benzoic acid (1.50 g, 4.63 mmol), (S)-methyl 2-aminopropanoate.HCl (0.78 g, 5.55 mmol) and HATU (2.3 g, 6.02 mmol) in dry DMF (15 mL) was added DIPEA (2.5 ml, 13.88 mmol). The reaciton was stirred at rt until complete by LC-MS. Next, the reaction mixture was poured into water (150 mL) and extracted with EtOAc (4×100 mL) before the combined organic extracts were washed with sat. aq. ammonium chloride (100 mL), water (5×50 mL) then brine (50 mL) and dried (MgSO₄), filtered and concentrated in vacuo. The crude material was purified by chromatography (25-100% EtOAc in iso-hexanes) to give (S)-methyl 2-(3-(6-(dimethylamino)-6-oxohex-1-yn-1-yl)benzamido)propanoate (1.17 g, 68.3% yield at 90% purity). Material of sufficient purity to proceed. A sample (100 mg) was purified by preparative HPLC (20-50% MeCN in water (0.1% formic)) to give analytically pure material.

δ(¹H) DMSO-d₆: 1.39 (3H, d, J=7.3 Hz), 1.77 (2H, qn, J=7.2 Hz), 2.43-2.48 (4H, m), 4.82 (3H, s), 2.97 (3H, s), 3.64 (3H, s), 4.47 (11-1, qn, J=7.32 Hz), 7.46 (1H, t, J=7.7 Hz), 7.56 (1H, td, J=1.3, 7.7 Hz), 7.82 (1H, td, J=1.5, 7.8 Hz), 7.91-7.94 (1H, m), 8.89 (1H, d, J=6.9 Hz) ppm.

δ(¹³C) DMSO-d₆: 16.68, 18.24, 23.94, 31.21, 34.80, 36.64, 48.31, 51.93, 80.20, 91.15, 123.22, 127.13, 128.78, 130.05, 133.90, 134.03, 165.43, 171.31 ppm.

MS(ES+) m/z 345 (M+H).

### (S)-methyl 2-(3-(6-(dimethylamino)-6-oxohexyl)benzamido)propanoate VSN 64

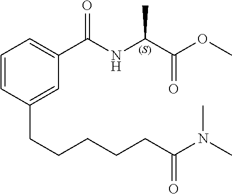

Following the general procedure for the reduction of an alkyne to alkane, the hydrogenation of (S)-methyl 2-(3-(6-(dimethylamino)-6-oxohex-1-yn-1-yl)benzamido)propanoate (0.37 g, 1.07 mmol) after purification by preparative HPLC (20-50% MeCN in water (0.1% formic)) gave the title compound (S)-methyl 2-(3-(6-(dimethylamino)-6-oxohexyl) benzamido)propanoate (228 mg, 59.7% yield) as a colourless viscous oil.

δ(¹H) DMSO-d₆: 1.26-1.35 (2H, m), 1.40 (3H, t, J=7.4 Hz), 1.48-1.55 (2H, m), 1.56-1.64 (2H, m), 2.26 (2H, t, J=7.4 Hz), 2.60-2.64 (2H, m), 2.79 (3H, s), 2.93 (3H, s), 3.64 (3H, s), 4.47 (1H, qn, J=7.2 Hz), 7.35-7.39 (2H, m), 7.66-7.71 (2H, m), 8.74 (1H, d, J=7.0 Hz) ppm.

δ(¹³C) DMSO-d₆: 16.73, 24.48, 28.44, 30.82, 32.24, 34.73, 34.97, 36.67, 48.22, 51.85, 124.90, 127.26, 128.16, 131.42, 133.62, 142.41, 166.31, 171.84, 173.20 ppm.

MS(ES+) m/z 349 (M+H).

### (S)-2-(3-(6-(dimethylamino)-6-oxohexyl)benzamido)propanoic acid VSN 65

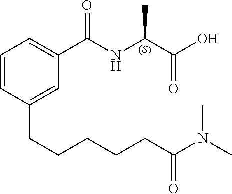

Following the general procedure for saponification, the reaction of (S)-methyl 2-(3-(6-(dimethylamino)-6-oxohexyl)benzamido)propanoate (0.10 g, 0.287 mmol) and lithium hydroxide (14 mg, 0.57 mmol) gave (S)-2-(3-(6-(dimethylamino)-6-oxohexyl) benzamido)propanoic acid (78 mg, 81% yield) as a white solid.

δ(¹H) DMSO-d₆: 1.25-1.35 (2H, m), 1.39 (3H, d, J=7.4 Hz), 1.46-1.56 (2H, m), 1.56-1.65 (2H, m), 2.26 (2H, t, J=7.4 Hz), 2.62 (2H, t, J=7.7 Hz), 2.79 (3H, s), 2.93 (3H, s), 4.41 (1H, qn, J=7.3 Hz), 7.36 (2H, dd, J=1.2, 4.0 Hz), 7.63-7.76 (2H, m), 8.60 (1H, d, J=7.2 Hz), 12.50 (1H, s) ppm.

δ(¹³C) DMSO-d₆: 16.89, 24.48, 28.44, 30.83, 32.25, 34.74, 34.99, 36.67, 48.08, 124.88, 127.24, 128.11, 131.29, 133.89, 142.36, 166.20, 171.85, 174.23 ppm.

MS(ES+) m/z 335 (M+H).

Synthesis of VSN 66-68

### N,N-dimethylpent-4-ynamide

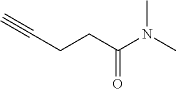

To a solution of pent-4-ynoic acid (3.1 g, 31.6 mmol) in dry DCM (30 mL) and DMF (1 drop) at 0° C. was added oxalyl chloride (4.01 ml, 47.4 mmol). The reaction was allowed to warm to rt and stir for 1 h before the volatiles were removed in vacuo. The residue was redissolved in dry THF (10 mL) and added drop-wise to a cooled (ice bath) solution of dimethylamine (40 wt % in water) (20.0 ml, 158 mmol). The reaction was stirred in the ice bath for 1 h then extracted with DCM (3×30 mL). The combined organic extracts were washed with water (50 mL) and dried (MgSO₄), filtered then concentrated in vacuo to give N,N-dimethylpent-4-ynamide (3.3 g, 79% yield) as a brown free-flowing oil that solidified on standing.

δ(¹H) DMSO-d₆: 2.31-1.35 (2H, m), 2.49-2.51 (2H, m), 2.74 (1H, t, J=2.6 Hz), 2.81 (3H, s), 2.94 (3H, s) ppm.

### (R)-methyl 2-(3-(5-(dimethylamino)-5-oxopent-1-yn-1-yl)benzamido)propanoate VSN 66

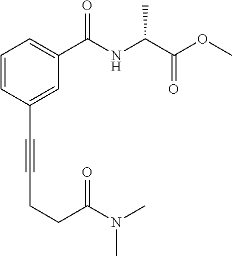 [SMILES: COC(=O)[C@@H](C)NC(=O)c1cccc(C#CCCC(=O)N(C)C)c1]

Following the general method for Sonogashira coupling, the reaction of (R)-methyl 2-(3-iodobenzamido)propanoate (2.0 g, 4.80 mmol) and N,N-dimethylpent-4-ynamide (0.73 g, 5.52 mmol) after purification by column chromatography (1-3% MeOH in DCM) gave (R)-methyl 2-(3-(5-(dimethylamino)-5-oxopent-1-yn-1-yl)benzamido)propanoate (1.4 g, 86% yield).

δ(¹H) DMSO-d₆: 1.40 (3H, d, J=7.3 Hz), 2.64 (4H, br s), 2.82 (3H, s), 2.99 (3H, s), 3.65 (3H, s), 4.48 (1H, qn, J=7.2 Hz), 7.46 (1H, t, J=7.8 Hz), 7.55 (1H, td, J=1.4, 7.7 Hz), 7.83 (1H, td, J=1.6, 7.9 Hz), 7.92 (1H, t, J=1.4 Hz), 8.89 (1H, d, J=6.9 Hz) ppm.

δ(¹³C) DMSO-d₆: 14.77, 16.66, 31.59, 34.89, 36.53, 48.28, 51.89, 79.63, 91.22, 123.19, 127.09, 128.74, 130.03, 133.92, 133.95, 165.42, 170.16, 173.05 ppm.

MS(ES+) m/z 331 (M+H).

### (R)-methyl 2-(3-(5-(dimethylamino)-5-oxopentyl)benzamido)propanoate VSN 67

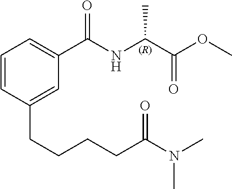

Following the general procedure for the reduction of an alkyne to alkane, the hydrogenation of (R)-methyl 2-(3-(5-(dimethylamino)-5-oxopent-1-yn-1-yl)benzamido)propanoate (400 mg, 1.211 mmol) after purification by chromatography (1-3% MeOH in DCM) gave the title compound (R)-methyl 2-(3-(5-(dimethylamino)-5-oxopentyl)benzamido)propanoate (0.36 g, 87% yield) as a colourless oil.

δ(¹H) DMSO-d₆: 1.40 (3H, d, J=7.3 Hz), 1.45-1.56 (2H, m), 1.56-1.69 (2H, m), 2.30 (2H, t, J=7.3 Hz), 2.64 (2H, t, J=7.5 Hz), 2.79 (3H, s), 2.93 (3H, s), 3.64 (3H, s), 4.32-4.73 (1H, m), 7.37 (2H, dd, J=1.0, 4.1 Hz) 7.58-7.87 (2H, m), 8.74 (1H, d, J=6.9 Hz) ppm.

δ(¹³C) DMSO-d₆: 16.74, 24.26, 30.53, 32.11, 34.74, 34.85, 36.67, 48.23, 51.85, 124.90, 127.25, 128.16, 131.42, 133.63, 142.32, 166.32, 171.79, 173.20 ppm.

MS(ES+) m/z 335 (M+H).

### (R)-2-(3-(5-(dimethylamino)-5-oxopentyl)benzamido)propanoic acid VSN 68

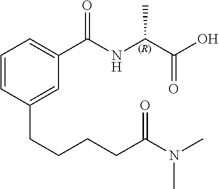

Following the general saponification procedure, the reaction of (R)-methyl 2-(3-(5-(dimethylamino)-5-oxopentyl)benzamido)propanoate (0.15 g, 0.45 mmol) with lithium hydroxide (16 mg, 0.67 mmol) gave (R)-2-(3-(5-(dimethylamino)-5-oxopentyl)benzamido)propanoic acid (82 mg, 55.9% yield) as a waxy white solid.

δ(¹H) DMSO-d₆: 1.39 (3H, d, J=7.4 Hz), 1.46-1.54 (2H, m), 1.58-1.65 (2H, m), 2.30 (2H, t, J=7.3 Hz), 2.64 (2H, t, J=7.5 Hz), 2.79 (3H, s), 2.93 (3H, s), 4.41 (1H, qn, J=7.3 Hz), 7.34-7.39 (2H, m), 7.65-7.74 (2H, m), 8.61 (1H, d, J=7.2 Hz), 12.54 (1H, br s) ppm.

δ(¹³C) DMSO-d₈: 16.91, 24.27, 30.55, 32.12, 34.75, 34.86, 36.68, 48.10, 124.88, 127.22, 128.12, 131.29, 133.91, 142.26, 166.20, 171.80, 174.23 ppm.

MS(ES+) Ink 321 (M+H).

Synthesis of VSN 69 to 71

### (R)-methyl 2-(3-(6-(dimethylamino)-6-oxohex-1-yn-1-yl)benzamido)propanoate VSN 69

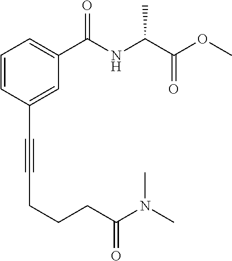 [SMILES: COC(=O)[C@@H](C)NC(=O)c1cccc(C#CCCCC(=O)N(C)C)c1]

Using the general procedure described for amide coupling, the reaction of (R)-6-(3-((1-methoxy-1-oxopropan-2-yl)carbamoyl)phenyl)hex-5-ynoic acid (2.41 g, 5.70 mmol), dimethylamine.HCl (0.56 g, 6.84 mmol), HATU (2.60 g, 6.84 mmol) and TEA (1.99 ml, 14.24 mmol) in dry DCM (30 mL) after purification by chromatography (25-100% EtOAc in iso-hexanes) gave the title compound (R)-methyl 2-(3-(6-(dimethylamino)-6-oxohex-1-yn-1-yl)benzamido)propanoate (1.94 g, 89% yield) as a colourless oil. A sample (150 mg) was purified by preparative HPLC (20-50% MeCN in water (0.1% formic)) to give analytically pure material (128 mg).

δ(¹H) DMSO-d₆: 1.40 (3H, d, J=7.3 Hz), 1.78 (2H, qn, J=7.1 Hz), 2.43-2.50 (4H, m), 2.83 (3H, s), 2.98 (3H, s), 3.65 (3H, s), 4.48 (1H, qn, J=7.3 Hz), 7.47 (1H, t, J=7.8 Hz), 7.57 (1H, td, J=1.3, 7.6 Hz), 7.83 (1H, td, J=1.4, 7.8 Hz), 7.93 (1H, t, J=1.5 Hz), 8.90 (1H, d, J=7.0 Hz) ppm.

δ(¹³C) DMSO-d₆: 16.69, 18.25, 23.95, 31.22, 34.81, 36.65, 48.31, 51.94, 91.16, 123.22, 127.13, 128.79, 130.06, 133.91, 134.05, 165.44, 171.32, 173.10 ppm.

MS(ES+) m/z 345 (M+H).

### (R)-methyl 2-(3-(6-(dimethylamino)-6-oxohexyl)benzamido)propanoate VSN 70

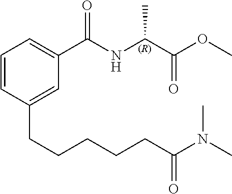

Following the general procedure for the reduction of an alkyne to alkane, the hydrogenation of (R)-methyl 2-(3-(6-(dimethylamino)-6-oxohex-1-yn-1-yl)benzamido)propanoate (250 mg, 0.73 mmol) after purification by preparative HPLC (20-50% MeCN in water (0.2% formic)) gave the title compound (R)-methyl 2-(3-(6-(dimethylamino)-6-oxohexyl) benzamido)propanoate (172 mg, 66.6% yield) as a colourless viscous oil.

δ(¹H) DMSO-d₆: 1.21-1.36 (2H, m), 1.40 (3H, d, J=7.3 Hz), 1.51 (2H, qn, J=7.4 Hz), 1.60 (2H, qn, J=7.6 Hz), 2.26 (2H, t, J=7.4 Hz), 2.57-2.68 (2H, m), 2.79 (3H, s), 2.93 (3H, s), 3.64 (3H, s), 4.47 (1H, qn, J=7.3 Hz), 7.37 (2H, d, J=5.0 Hz), 7.65-7.73 (2H, m), 8.74 (1H, d, J=6.9 Hz) ppm.

δ(¹³C) DMSO-d₆: 16.73, 24.48, 28.44, 30.82, 32.24, 34.73, 34.98, 36.67, 48.22, 51.85, 124.90, 127.26, 128.16, 131.42, 133.62, 142.42, 166.31, 171.85, 173.20 ppm.

MS(ES+) m/z 349 (M+H).

### (R)-2-(3-(6-(dimethylamino)-6-oxohexyl)benzamido)propanoic acid VSN 71

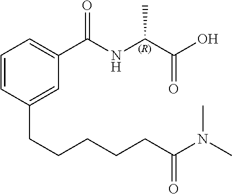

Following the general procedure for the reduction of an alkyne to alkane, the hydrogenation of (R)-2-(3-(6-(dimethylamino)-6-oxohex-1-yn-1-yl)benzamido)propanoic acid (146 mg, 0.44 mmol) gave the title compound (R)-2-(3-(6-(dimethylamino)-6-oxohexyl) benzamido)propanoic acid (0.14 g, 93% yield) as a colourless viscous oil. No purification required.

δ(¹H) DMSO-d₆: 1.26-1.36 (2H, m), 1.40 (3H, d, J=7.4 Hz), 1.48-1.57 (2H, m), 1.57-1.66 (2H, m), 2.27 (2H, t, J=7.4 Hz), 2.59-2.67 (2H, m), 2.79 (3H, s), 2.94 (3H, s), 4.41 (1H, qn, J=7.4 Hz), 7.32-7.42 (2H, m), 7.65-7.76 (2H, m), 8.60 (1H, d, J=7.2 Hz), 12.51 (1H, s) ppm.

δ(¹³C) DMSO-d₆: 16.93, 24.53, 28.49, 30.90, 32.28, 34.77, 35.04, 36.70, 48.14, 54.95, 124.92, 127.28, 128.16, 131.35, 133.90, 142.40, 166.23, 171.89, 174.31 ppm.

MS(ES+) m/z 335 (M+H).

Synthesis of VSN 72 to 74

### oct-7-ynoic acid

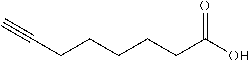

To a solution of 6-bromohexanoic acid (2.4 g, 12.30 mmol) in dry DMSO (20 mL) under nitrogen was was added lithium acetylide ethylenediamine complex (2.49 g, 27.1 mmol) protion-wise over 15 min. Upon complete addition, the resulting brown solution was stirred at rt for 2 h. The reaction was then quenched by the addition of water (20 mL) and acidified to pH 1 with 1 N HCl. The product was then extracted with EtOAc (4×60 mL) before the combined organic extracts were washed with water (5×80 mL) and brine (60 mL) then dried (MgSO4), filtered and concentrated in vacuo to give oct-7-ynoic acid (0.7 g, 36.5% yield) as a pale orange oil.

δ(¹H) DMSO-d₆: 1.30-1.38 (2H, m), 1.39-1.46 (2H, m), 1.39-1.54 (2H, m), 2.14 (2H, dt, J=2.7, 7.0 Hz), 2.20 (2H, t, J=7.4 Hz), 2.75 (1H, t, J=2.7 Hz), 11.98 (1H, s) ppm.

### (R)-8-(3-((1-methoxy-1-oxopropan-2-yl)carbamoyi)phenyl)oct-7-ynoic acid

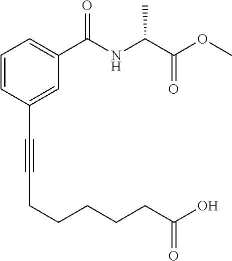 [SMILES: COC(=O)[C@@H](C)NC(=O)c1cccc(C#CCCCCCC(=O)O)c1]

Following the general method for Sonogashira coupling, the reaction of (R)-methyl 2-(3-iodobenzamido)propanoate (1.8 g, 4.34 mmol) and oct-7-ynoic acid (0.7 g, 4.99 mmol) after purification by column chromatography (1-3% MeOH in DCM) gave (R)-8-(3-((1-methoxy-1-oxopropan-2-yl)carbamoyl)phenyl)oct-7-ynoic acid (1.3 g, 69.3% yield@80% purity).

δ(¹H) DMSO-d₆: 1.35-1.49 (5H, m), 1.50-1.63 (4H, m), 2.23 (2H, t, J=7.2 Hz), 2.44 (2H, t, J=7.0 Hz), 3.64 (3H, s), 4.47 (1H, qn, J=7.3, 1.8 Hz), 7.44-7.50 (1H, m), 7.55 (1H, td, J=1.4, 7.7 Hz), 7.82 (1H, dt, J=1.5, 7.8 Hz), 7.86-7.95 (1H, m), 8.83-8.90 (1H, m), 12.00 (1H, s) ppm.

MS(ES+) m/z 346 (M+H).

### (R)-methyl 2-(3-(8-(dimethylamino)-8-oxooct-1-yn-1-yl)benzamido)propanoate VSN 72

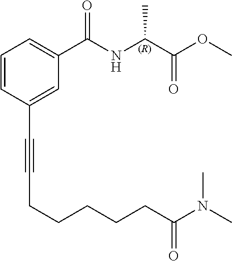

Using the general procedure described for amide coupling, the reaction of (R)-8-(3-((1-methoxy-1-oxopropan-2-yl)carbamoyl)phenyl)oct-7-ynoic acid (1.3 g, 3.01 mmol), dimethylamine.HCl (0.27 g, 3.31 mmol), DIPEA (1.32 ml, 7.53 mmol) and HATU (1.32 g, 3.46 mmol) in dry DCM (15 mL) after purification by chromatography (1-3% MeOH in DCM) gave the title compound (R)-methyl 2-(3-(8-(dimethylamino)-8-oxooct-1-yn-1-yl)benzamido)propanoate (1.0 g, 85% yield) as a viscous yellow oil.

δ(¹H) DMSO-d₆: 1.39 (3H, d, J=7.3 Hz), 1.42-1.47 (2H, m), 1.48-1.61 (4H, m), 2.30 (2H, t, J=7.5 Hz), 2.44 (2H, t, J=7.0 Hz), 2.79 (3H, s), 2.95 (3H, s), 3.64 (3H, s), 4.47 (1H, qn, J=7.2 Hz), 7.45 (1H, t, J=7.8 Hz), 7.55 (1H, td, J=1.4, 7.8 Hz), 7.81 (1H, td, J=1.6, 7.9 Hz), 7.91 (1H, t, J=1.5 Hz), 8.87 (1H, d, J=6.9 Hz) ppm.

δ(¹³C) DMSO-d₆: 16.70, 18.52, 24.18, 27.95, 28.13, 32.19, 34.73, 36.67, 48.27, 51.87, 79.93, 91.45, 123.29, 127.02, 128.71, 129.96, 133.89, 133.97, 165.41, 171.81, 173.04 ppm.

MS(ES+) m/z 373 (M+H).

### (R)-methyl 2-(3-(8-(dimethylamino)-8-oxooctyl)benzamido)propanoate VSN 73

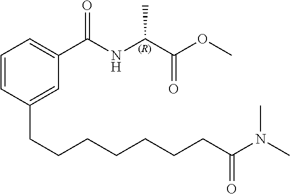

Following the general procedure for the reduction of an alkyne to alkane, the hydrogenation of (R)-methyl 2-(3-(8-(dimethylamino)-8-oxooct-1-yn-1-yl)benzamido)propanoate (400 mg, 1.07 mmol) after purification by chromatography (1-5% MeOH in DCM) gave the title compound (R)-methyl 2-(3-(8-(dimethylamino)-8-oxooctyl)benzamido)propanoate (0.34 g, 80% yield) as a colourless oil.

δ(¹H) DMSO-d₆: 1.24-1.29 (6H, m), 1.40 (3H, d, J=7.32 Hz), 1.46 (2H, qn, J=6.9 Hz), 1.55-1.62 (2H, m), 2.24 (2H, t, J=7.5 Hz), 2.60-2.64 (2H, m), 2.79 (3H, s), 2.93 (3H, s), 3.64 (3H, s), 4.47 (1H, qn, J=7.2 Hz), 7.35-7.39 (2H, m), 7.66-7.72 (2H, m), 8.74 (1H, d, J=7.0 Hz) ppm.

δ(¹³C) DMSO-d₆: 16.73, 24.62, 28.58, 28.69, 28.73, 30.89, 32.28, 34.72, 35.02, 36.67, 48.22, 51.84, 124.87, 127.23, 128.14, 131.39, 133.62, 142.48, 166.31, 171.89, 173.20 ppm.

MS(ES+) m/z 377 (M+H).

### (R)-2-(3-(8-(dimethylamino)-8-oxooctyl)benzamido)propanoic acid VSN 74

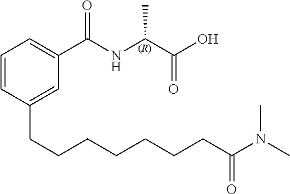

Following the general procedure for saponification, the reaction of (R)-methyl 2-(3-(8-(dimethylamino)-8-oxooctyl)benzamido)propanoate (0.15 g, 0.40 mmol) with lithium hydroxide (14 mg, 0.60 mmol) gave (R)-2-(3-(8-(dimethylamino)-8-oxooctyl) benzamido)propanoic acid (0.11 g, 74.6% yield).

δ(¹H) DMSO-d₆: 1.22-1.34 (6H, m), 1.40 (3H, d, J=7.4 Hz), 1.47 (2H, qn, J=7.4 Hz), 1.56-1.63 (2H, m), 2.25 (2H, t, J=7.4 Hz), 2.61-2.65 (2H, m), 2.79 (3H, s), 2.94 (3H, s), 4.42 (1H, qn, J=7.3 Hz), 7.35-7.39 (2H, m), 7.68-7.72 (2H, m) 8.61 (1H, d, J=7.3 Hz), 12.52 (1H, s) ppm.

δ(¹³C) DMSO-d₆: 16.89, 24.63, 28.60, 28.70, 28.74, 30.91, 32.29, 34.73, 35.04, 36.68, 48.08, 124.86, 127.21, 128.10, 131.26, 133.89, 142.42, 166.21, 171.90, 174.24 ppm.

MS(ES+) m/z 363 (M+H).

Synthesis of VSN 75 to 77

### methyl 3-(3-(6-(dimethylamino)-6-oxohex-1-yn-1-yl)benzamido)propanoate VSN 75

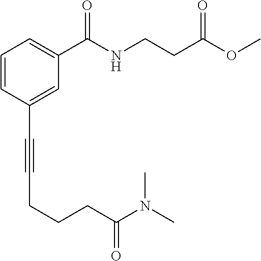 [SMILES: COC(=O)CCNC(=O)c1cccc(C#CCCCC(=O)N(C)C)c1]

Using the general procedure described for amide coupling, the reaction of 3-(6-(dimethylamino)-6-oxohex-1-yn-1-yl)benzoic acid (0.75 g, 2.314 mmol), methyl 3-aminopropanoate.HCl (0.36 g, 2.55 mmol), DIPEA (1.24 ml, 6.94 mmol) and HATU (1.14 g, 3.01 mmol) in dry DCM (15 mL) after purfication by chromatography (1-3% MeOH in DCM) gave the title compound methyl 3-(3-(6-(dimethylamino)-6-oxohex-1-yn-1-yl)benzamido)propanoate (0.66 g, 79% yield) as a viscous pale yellow oil.

δ(¹H) DMSO-d₆: 1.78 (2H, qn, J=7.2 Hz), 2.41 2.48 (4H, m), 2.59 (2H, t, J=7.0 Hz), 2.82 (3H, s), 2.97 (3H, s), 3.43-3.52 (2H, m), 3.60 (3H, s), 7.44 (1H, t, J=7.8 Hz), 7.53 (1H, td, J=1.3, 7.7 Hz), 7.77 (1H, td, J=1.4, 7.8 Hz), 7.84 (1H, t, J=1.5 Hz), 8.61 (1H, t, J=5.4 Hz) ppm.

δ(¹³C) DMSO-d₆: 18.22, 23.93, 31.19, 33.41, 34.77, 35.51, 36.62, 51.38, 80.23, 91.04, 123.17, 126.81, 128.70, 129.77, 133.72, 134.55, 165.45, 171.29, 171.70 ppm.

MS(ES+) m/z 345 (M+H).

### (Z)-methyl 3-(3-(6-(dimethylamino)-6-oxohex-1-en-1-yl)benzamido)propanoate VSN 76

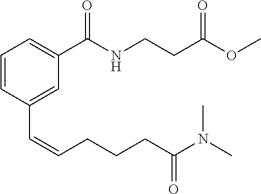 [SMILES: COC(=O)CCNC(=O)c1cccc(/C=C\CCCC(=O)N(C)C)c1]

Following the general procedure for the Lindlar reduction, the hydrogenation of methyl 3-(3-(6-(dimethylamino)-6-oxohex-1-yn-1-yl)benzamido)propanoate (0.40 g, 1.16 mmol) gave the named product along with the trans double bond isomer (7%) and fully saturated product (25%) (determined by ¹H NMR). Separation by column chromatography (1-3% MeOH in DCM) gave the title compound (0.20 g, 48.7% yield). The other 2 components were not isolated.

δ(¹H) DMSO-d₆: 1.64 (2H, qn, J=7.4 Hz), 2.25-2.34 (4H, m), 2.60 (2H, t, J=7.0 Hz), 2.78 (3H, s), 2.92 (3H, s), 3.45-3.54 (2H, m), 3.60 (3H, s), 5.73 (1H, td, J=7.3, 11.7 Hz), 6.46 (1H, br d, J=11.7 Hz), 7.43 (2H, dd, J=1.3, 3.9 Hz), 7.64-7.75 (2H, m), 8.57 (1H, t, J=5.4 Hz) ppm.

δ(¹³C) DMSO-d₆: 24.75, 27.72, 31.71, 33.54, 34.74, 35.51, 36.61, 51.36, 125.42, 127.16, 128.25, 128.36, 131.11, 133.22, 134.39, 137.08, 166.22, 171.63, 171.73 ppm.

MS(ES+) m/z 347 (M+H).

### (Z)-3-(3-(6-(dimethylamino)-6-oxohex-1-en-1-yl)benzamido)propanoic acid VSN 77

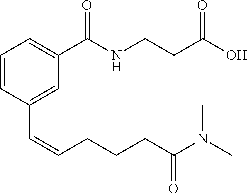 [SMILES: CN(C)C(=O)CCC/C=C\c1cccc(C(=O)NCCC(=O)O)c1]

Following the general procedure for saponification, the reaction of (Z)-methyl 3-(3-(6-(dimethylamino)-6-oxohex-1-en-1-yl)benzamido)propanoate (0.1 g, 0.29 mmol) with lithium hydroxide (14 mg, 0.58 mmol) gave (Z)-3-(3-(6-(dimethylamino)-6-oxohex-1-en-1-yl)benzamido)propanoic acid (86 mg, 88% yield) as a colourless gum.

δ(¹H) DMSO-d₆: 1.64 (2H, qn, J=7.4 Hz), 2.27-2.32 (4H, m), 2.50-2.55 (2H, m), 2.78 (3H, s), 2.92 (3H, s), 3.46 (2H, q, J=7.1 Hz), 5.73 (1H, dt, J=7.3, 11.7 Hz), 6.46 (1H, d, J=11.7 Hz), 7.43 (2H, dd, J=3.9, 1.4 Hz), 7.67-7.71 (1H, m), 7.72 (1H, br s), 8.55 (1H, t, J=5.5 Hz), 12.20 (1H, s) ppm.

δ(¹³C) DMSO-d₆: 24.76, 27.73, 31.72, 33.76, 34.75, 35.59, 36.63, 125.41, 127.18, 128.24, 128.38, 131.07, 133.21, 134.45, 137.08, 166.14, 171.66, 172.86 ppm.

MS(ES+) m/z 333 (M+H).

Synthesis of VSN 78 to 80

### (R)-methyl 2-(3-(5-cyanopent-1-yn-1-yl)benzamido)propanoate VSN 78

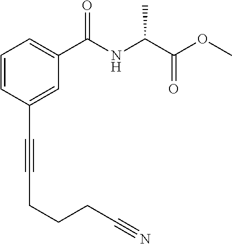 [SMILES: COC(=O)[C@@H](C)NC(=O)c1cccc(C#CCCCC#N)c1]

Following the general method for Sonogashira coupling, the reaction of (R)-methyl 2-(3-iodobenzamido)propanoate (1.6 g, 4.56 mmol) and hex-5-ynenitrile (0.72 ml, 6.84 mmol) after purification by chromatography (20-40% EtOAc in iso-hexanes) gave (R)-methyl 2-(3-(5-cyanopent-1-yn-1-yl)benzamido)propanoate (1.3 g, 93% yield).

δ(¹H) DMSO-d₆: 1.39 (3H, d, J=7.3 Hz), 1.86 (2H, qn, J=7.1 Hz), 2.57 (2H, t, J=7.0 Hz), 2.65 (2H, t, J=7.2 Hz), 3.64 (3H, s), 4.47 (1H, qn, J=7.2 Hz), 7.47 (1H, t, J=7.8 Hz), 7.58-7.60 (1H, m), 7.82-7.85 (1H, m), 7.94 (1H, t, J=1.4 Hz), 8.87 (1H, d, J=6.9 Hz) ppm.

δ(¹³C) DMSO-d₆: 15.58, 16.66, 17.86, 24.06, 48.29, 51.90, 80.88, 89.35, 120.18, 122.90, 127.27, 128.73, 130.11, 133.93, 134.10, 165.40, 173.05 ppm.

MS(ES+) m/z 299.2 (M+H).

### (R,Z)-methyl 2-(3-(5-cyanopent-1-en-1-yl)benzamido)propanoate VSN 79

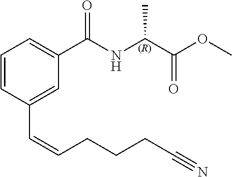

Following the general procedure for the Lindlar reduction, the hydrogenation of (R)-methyl 2-(3-(5-cyanopent-1-yn-1-yl)benzamido)propanoate (0.50 g, 1.68 mmol) gave the named product with trace amounts of the trans double bond isomer and fully saturated products (determined by ¹H NMR). Separation by column chromatography (1-2% MeOH in DCM) gave the title compound (0.42 g, 82% yield). The other 2 components were not isolated.

δ(¹H) DMSO-d₆: 1.41 (3H, d, J=7.3 Hz), 1.73 (2H, qn, J=7.3 Hz), 2.40 (2H, dq, J=1.8, 7.1 Hz), 2.53-2.55 (2H, m), 3.65 (31-1, s), 4.49 (1H, qn, J=7.2 Hz), 5.74 (1H, td, J=7.2, 11.6 Hz), 6.53 (1H, dt, J=1.6, 11.7 Hz), 7.47-7.49 (2H, m), 7.76-7.78 (2H, m), 8.81 (1H, d, J=6.9 Hz) ppm.

δ(¹³C) DMSO-d₆: 15.80, 16.72, 24.97, 27.10, 48.26, 51.87, 120.45, 125.83, 127.65, 128.30, 129.14, 131.28, 131.61, 133.81, 136.85, 166.13, 173.14 ppm.

MS(ES+) m/z 301.2 (M+H).

### (R,Z)-2-(3-(5-cyanopent-1-en-1-yl)benzamido)propanoic acid VSN 80

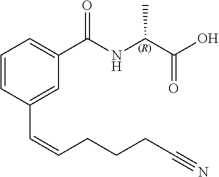

Following the general procedure for saponification, the reaction of (R,Z)-methyl 2-(3-(5-cyanopent-1-en-1-yl)benzamido)propanoate (0.15 g, 0.50 mmol) with lithium hydroxide (24 mg, 0.99 mmol) gave (R,Z)-2-(3-(5-cyanopent-1-en-1-yl)benzamido)propanoic acid (0.12 g, 80% yield).

δ(¹H) DMSO-d₆: 1.39 (3H, d, J=7.4 Hz), 1.72 (2H, qn, J=7.6 Hz), 2.39 (2H, dq, J=1.7, 7.3 Hz), 2.52-2.54 (2H, m), 4.41 (1H, qn, 7.3 Hz), 5.73 (1H, td, J=7.1, 11.7 Hz), 6.53 (1H, br d, J=11.7 Hz), 7.46-7.47 (2H, m), 7.76-7.77 (2H, m), 8.67 (1H, d, J=7.2 Hz), 12.53 (1H, br s) ppm.

δ(¹³C) DMSO-d₈: 15.80, 16.87, 24.98, 27.11, 48.14, 120.46, 125.81, 127.65, 128.26, 129.19, 131.16, 131.57, 134.08, 136.81, 166.02, 174.19 ppm.

MS(ES+) m/z 287.2 (M+H).

Synthesis of VSN 81, 85 and 86

### methyl 3-(5-hydroxypent-1-yn-1-yl)benzoate

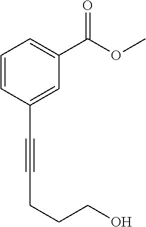 [SMILES: COC(=O)c1cccc(C#CCCCO)c1]

Following the general method for Sonogashira coupling, the reaction of methyl 3-iodobenzoate (7.45 g, 28.4 mmol) and pent-4-yn-1-ol (3.97 ml, 42.6 mmol) after purification by chromatography (10-50% EtOAc in iso-hexanes) gave methyl 3-(5-hydroxypent-1-yn-1-yl)benzoate (5.1 g, 81% yield) as a free flowing orange oil.

δ(¹H) DMSO-d₆: 1.63-1.75 (2H, m), 2.45-2.49 (2H, m), 3.46-3.57 (2H, m), 3.86 (3H, s), 4.55 (1H, t, J=5.2 Hz), 7.47-7.56 (1H, m), 7.65 (1H, td, J=1.4, 7.7 Hz), 7.86-7.93 (2H, m) ppm.

MS(ES+) m/z 219 (M+H).

### methyl 3-(5-(acetylthio)pent-1-yn-1-yl)benzoate

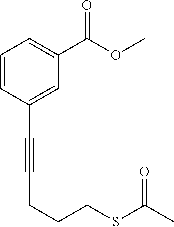 [SMILES: COC(=O)c1cccc(C#CCCCSC(C)=O)c1]

To a solution of methyl 3-(5-hydroxypent-1-yn-1-yl)benzoate (1.93 g, 8.84 mmol) and triethylamine (1.54 ml, 11.05 mmol) in dry DCM (20 mL) under nitrogen and cooled in an ice bath was added methanesulfonyl chloride (0.75 ml, 9.73 mmol) over 5 mins. The reaction mixture was then stirred at rt until judged complete by LCMS analysis. The reaction mixture was partitioned with DCM (50 mL) and sat. aq. ammonium chloride (30 mL). The aqueous layer was extracted with DCM (2×15 mL) before the combined organic extracts were washed with water (20 mL) and brine (20 mL) then dried (MgSO₄), filtered and concentrated in vacuo. The residue was then redissolved in dry DMF (25 mL) and treated with potassium ethanethloate (1.01 g, 8.84 mmol) and stirred at rt until judged complete by LCMS analysis. The reaction mixture was then partitioned between EtOAc (100 mL) and water (100 mL). The aqueous layer was extracted with EtOAc (3×30 mL) before the combined organic extracts were washed with water (5×30 mL), brine (30 mL) then dried (MgSO₄), filtered and concentrated in vacuo. The crude material was purified by chromatography (0-10% EtOAc in iso-hexanes) to give methyl 3-(5-(acetylthio)pent-1-yn-1-yl)benzoate (2.2 g, 85% yield) as an orange free-flowing oil.

δ(¹H) DMSO-d₆: 1.74-1.86 (2H, m), 2.34 (3H, s), 2.51-2.54 (2H, m), 2.95-3.03 (2H, m), 3.86 (3H, s), 7.49-7.55 (1H, m), 7.65-7.70 (1H, m), 7.89-7.94 (2H, m) ppm.

MS(ES+) m/z 277 (M+H).

### 5-(3-(methoxycarbonyl)phenyl)pent-4-yne-1-sulfonic acid

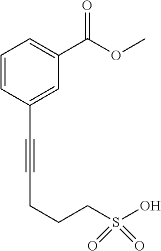 [SMILES: COC(=O)c1cccc(C#CCCCS(=O)(=O)O)c1]

Procedure followed from PCT 2003035627.

To a solution of methyl 3-(5-(acetylthio)pent-1-yn-1-yl)benzoate (2.2 g, 7.96 mmol) in AcOH (10 mL) was added a solution of hydrogen peroxide (9.76 ml, 127 mmol) in AcOH (20 mL). The reaction mixture was stirred at rt until complete by LC-MS analysis then cooled in an ice bath and quenched by the addition of 5% Pd/C (200 mg). The mixture was stirred for 20 min then filtered through a pad of celite and the volatiles were removed in vacuo. The residue was then azeoptroped with toluene (3×20 mL) to give 5-(3-(methoxycarbonyl)phenyl)pent-4-yne-1-sulfonic acid (1.9 g, 80%) as a brown semi-solid.

MS(ES+) m/z 281 (M+H).

### methyl 3-(5-(N,N-dimethylsulfamoyl)pent-1-yn-1-yl)benzoate

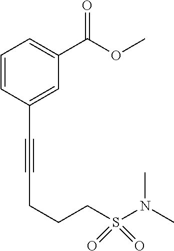 [SMILES: COC(=O)c1cccc(C#CCCCS(=O)(=O)N(C)C)c1]

A solution of 5-(3-(methoxycarbonyl)phenyl)pent-4-yne-1-sulfonic acid (1.9 g, 6.39 mmol) in dry DCM (30 mL) and dry DMF (2 drops) was treated with oxalyl dichloride (7.0 ml, 83.18 mmol) in 3 portions. The reaction was stirred at rt until judged complete by LCMS analysis. The volatiles were removed in vacuo and the residue was placed under nitrogen and redissolved in dry THF (10 mL) then treated with dimethylamine (2.0 M in THF) (32.0 ml, 63.9 mmol). The reaction mixture was stirred at rt until complete by LCMS then partitioned between EtOAc (100 mL) and 10% aq. citric acid (100 mL). The aqueous layer was extracted with EtOAc (2×100 mL) before the combined organic extracts were washed sequentially with 10% aq. citric acid ((50 mL), sat. aq. sodium bicarbonate (50 mL), water (50 mL) and brine (50 mL) then dried (MgSO₄), filtered and concentrated in vacuo. Purification was achieved by chromatography (10-50% EtOAc in iso-hexanes) to give methyl 3-(5-(N,N-dimethylsulfamoyl)pent-1-yn-1-yl)benzoate (1.6 g, 76% yield) as a pale yellow free-flowing oil.

δ(¹H) DMSO-d₆: 1.90-1.95 (2H, m), 2.61 (2H, t, J=7.1 Hz), 2.79 (6H, s), 3.14-3.22 (2H, m), 3.86 (3H, s), 7.52 (1H, dt, J=0.9, 7.6 Hz), 7.65-7.73 (1H, m), 7.89-7.95 (2H, m) ppm.

MS(ES+) m/z 310 (M+H).

3-(5-(N,N-dimethylsulfamoyl)pent-1-yn-1-yl)benzoic acid

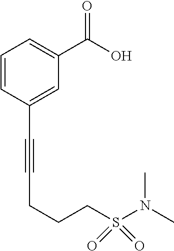 [SMILES: CN(C)S(=O)(=O)CCCC#Cc1cccc(C(=O)O)c1]

To a solution of methyl 3-(5-(N,N-dimethylsulfamoyl)pent-1-yn-1-yl)benzoate (1.60 g, 4.86 mmol) in THF (10 mL) was added a solution of lithium hydroxide (0.23 g, 9.72 mmol) in water (6 mL). The reaction mixture was stirred at rt until judged complete by LC-MS then the volatiles were removed in vacuo. The residue as diluted with water (10 mL) and acidifed to pH 1 with 1 N HCl (aq.). The resulting white precipitate was collected by filtration and washed with water (2×10 mL), dried by suction for 15 min then in a vacuum oven (40° C.) for 18 h to give 3-(5-(N,N-dimethylsulfamoyl)pent-1-yn-1-yl)benzoic acid (1.27 g, 87% yield) as a white solid.

δ(¹H) DMSO-d₆: 1.87-2.02 (2H, m), 2.61 (211, t, J=7.0 Hz), 2.79 (6H, s), 3.13-3.25 (2H, m), 7.49 (1H, dt, J=0.8, 7.6 Hz), 7.65 (1H, td, J=1.5, 7.7 Hz), 7.86-7.94 (2H, m), 13.16 (1H, s) ppm.

MS(ES+) m/z 296 (M+H).

### (R)-methyl 2-(3-(5-(N,N-dimethylsulfamoyl)pent-1-yn-1-yl)benzamido)propanoate VSN 81

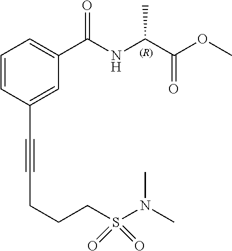

Using the general procedure described for amide coupling, the reaction of 3-(5-(N,N-dimethylsulfamoyl)pent-1-yn-1-yl)benzoic acid (0.81 g, 2.74 mmol), (R)-methyl 2-aminopropanoate.HCl (0.421 g, 3.02 mmol), DIPEA (1.20 ml, 6.86 mmol) and HATU (1.20 g, 3.15 mmol) in dry DCM (20 mL) after purification by chromatography (10-100% EtOAc in iso-hexanes) gave the title compound (R)-methyl 2-(3-(5-(N,N-dimethylsulfamoyl)pent-1-yn-1-yl)benzamido)propanoate (1.0 g, 93% yield) as a yellow oil

δ(¹H) DMSO-d₆: 1.40 (3H, d, J=7.3 Hz), 1.96 (2H, dd, J=6.2, 13.9 Hz), 2.62 (2H, t, J=7.1 Hz), 2.79 (6H, s), 3.12-3.24 (2H, m), 3.64 (3H, s), 4.47 (1H, qn, J=7.2 Hz), 7.47 (1H, t, J=7.7 Hz), 7.58 (1H, d, J=7.7 Hz), 7.83 (1H, d, J=7.9 Hz), 7.93 (1H, s), 8.87 (1H, d, J=7.0 Hz) ppm.

δ(¹³C) DMSO-d₆: 16.66, 17.53, 22.20, 37.07, 45.52, 48.28, 51.89, 80.70, 89.86, 122.92, 127.25, 128.76, 130.11, 133.94, 134.07, 165.40, 173.04 ppm.

MS(ES+) m/z 381 (M+H).

### (R,Z)-methyl 2-(3-(5-(N,N-dimethylsulfamoyl)pent-1-en-1-yl)benzamido)propanoate VSN 85

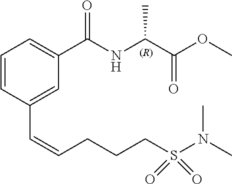

Following the general procedure for the Lindlar reduction, the hydrogenation of (R)-methyl 2-(3-(5-(N,N-dimethylsulfamoyl)pent-1-yn-1-yl)benzamido)propanoate (0.50 g, 1.31 mmol) after separation by column chromatography (0-2% MeOH in DCM) then repurification by column chromatography (10% EtOAc in DCM) and prep. HPLC (30% MeCN in water, acidic) gave the title compound (R,Z)-methyl 2-(3-(5-(N,N-dimethylsulfamoyl)pent-1-en-1-yl)benzamido)propanoate (0.13 g, 24% yield@95% purity) as a colourless oil.

δ(¹H) DMSO-d₆: 1.40 (3H, d, J=7.3 Hz), 1.77-1.87 (2H, m), 2.43 (2H, dq, J=1.9, 7.4 Hz), 2.74 (6H, s), 3.03-3.09 (2H, m), 3.65 (3H, s), 4.48 (1H, qn, J=7.3 Hz), 5.75 (1H, td, J=7.2, 11.7 Hz), 6.52 (1H, td, J=1.9, 11.7 Hz), 7.44-7.49 (2H, m), 7.75-7.78 (2H, m), 8.80 (1H, d, J=6.9 Hz) ppm.

δ(¹³C) DMSO-d₆: 16.73, 22.86, 26.78, 37.09, 45.79, 48.29, 51.91, 125.82, 127.68, 128.35, 128.96, 131.35, 132.13, 133.80, 136.94, 166.17, 173.18 ppm.

MS(ES+) m/z 383 (M+H).

### (R,Z)-2-(3-(5-(N,N-dimethylsulfamoyl)pent-1-en-1-yl)benzamido)propanoic acid VSN 86

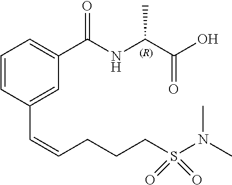

Following the general procedure for saponification, the reaction of (R,Z)-methyl 2-(3-(5-(N,N-dimethylsulfamoyl)pent-1-en-1-yl)benzamido)propanoate (0.075 g, 0.20 mmol) with lithium hydroxide (9.4 mg, 0.39 mmol) gave (R,Z)-2-(3-(5-(N,N-dimethylsulfamoyl)pent-1-en-1-yl)benzamido)propanoic acid (68 mg, 89% yield) as a white solid.

δ(¹H) DMSO-d₆: 1.40 (3H, d, J=7.4 Hz), 1.75-1.85 (2H, m), 2.39-2.48 (2H, m), 2.75 (6H, s), 3.02-3.11 (2H, m), 4.36-4.47 (1H, m), 5.76 (1H, td, J=7.2, 11.7 Hz), 6.53 (1H, br d, J=11.7 Hz), 7.44-7.51 (2H, m), 7.76-7.79 (2H, m), 8.70 (1H, d, J=7.2 Hz), 12.57 (1H, br s) ppm.

δ(¹³C) DMSO-d₆: 16.89, 22.87, 26.81, 37.11, 45.79, 48.18, 125.81, 127.69, 128.32, 129.02, 131.24, 132.09, 134.06, 136.91, 166.06, 174.25 ppm.

MS(ES+) m/z 369 (M+H).

Synthesis of VSN 82, 87 and 88

### methyl 3-(5-(2-oxopyridin-1(2H)-yl)pent-1-yn-1-yl)benzoate

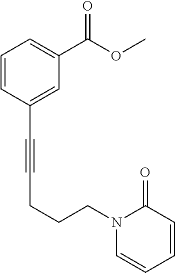 [SMILES: COC(=O)c1cccc(C#CCCCn2ccccc2=O)c1]

A suspension of methyl 3-(5-((methylsulfonyl)oxy)pent-1-yn-1-yl)benzoate (1.15 g, 3.88 mmol), pyridin-2(1H)-one (0.41 g, 4.27 mmol) and potassium carbonate (1.07 g, 7.76 mmol) in dry MeCN (12 mL) under nitrogen was heated to 60° C. for 18 h. Then the mixture was allowed to cool to rt before it was partitioned between EtOAc (50 mL) and water (50 mL). The aqueous layer was extracted with EtOAc (2×30 mL) and the combined organic extracts were washed with water (30 mL) and brine (30 mL) then dried (MgSO₄), filtered and concentrated in vacuo. Purification was achieved by chromatography (0-100% EtOAc in iso-hexanes) to give the title product methyl 3-(5-(2-oxopyridin-1(2H)-yl)pent-1-yn-1-yl)benzoate (0.63 g, 53.9% yield) as a clear colourless oil.

δ(¹H) DMSO-d₆: 1.93 (2H, qn, J=7.1 Hz), 2.47 (2H, t, J=7.1 Hz), 3.86 (3H, s), 3.97-4.04 (2H, m), 6.21 (1H, dt, J=1.4, 6.7 Hz), 6.35-6.40 (1H, m), 7.39 (1H, ddd, J=2.1, 6.6, 8.9 Hz), 7.47-7.54 (1H, m), 7.66 (1H, td, J=1.5, 7.8 Hz), 7.69 (1H, ddd, J=0.7, 2.1, 6.8 Hz), 7.88-7.91 (2H, m) ppm.

δ(¹³C) DMSO-d₆: 16.08, 27.39, 30.65, 47.98, 52.30, 79.85, 90.70, 105.18, 119.62, 123.62, 128.50, 129.15, 129.97, 131.74, 135.77, 139.18, 139.84, 161.45, 165.54 ppm.

MS(ES+) m/z 296 (M+H).

### 3-(5-(2-oxopyridin-1(2H)-yl)pent-1-yn-1-yl)benzoic acid

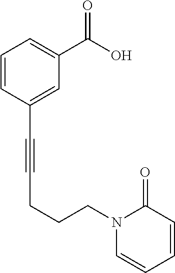 [SMILES: O=C(O)c1cccc(C#CCCCn2ccccc2=O)c1]

To a solution of methyl 3-(5-(2-oxopyridin-1(2H)-yl)pent-1-yn-1-yl)benzoate (0.63 g, 2.13 mmol) in THF (10 mL) was added a solution of lithium hydroxide (0.10 g, 4.27 mmol) in water (3.0 mL). The resulting mixture was stirred at rt for 16 h before the volatiles were removed in vacua The residue was then partitioned between 1 N HCl (30 mL) and EtOAc (50 mL) and the aqueous layer was extracted with EtOAc (2×40 mL). The combined organic extracts were washed with water (30 mL) and brine (30 mL) then dried (MgSO₄), filtered and concentrated in vauco to give the title compound 3-(5-(2-oxopyridin-1(2H)-yl)pent-1-yn-1-yl)benzoic acid (0.6 g, 98% yield) as an off-white solid.

δ(¹H) DMSO-d₆: 1.93 (2H, qn, J=7.0 Hz), 2.43-2.49 (2H, m), 4.01 (2H, t, J=7.1 Hz), 6.21 (1H, dt, J=1.3, 6.7 Hz), 6.34-6.42 (11-1, m), 7.39 (1H, ddd, J=2.1, 6.6, 8.9 Hz), 7.45-7.52 (1H, m), 7.63 (1H, td, J=1.4, 7.7 Hz), 7.69 (1H, dd, J=1.6, 6.7 Hz), 7.85-7.93 (2H, m), 13.15 (1H, s) ppm.

MS(ES+) m/z 282 (M+H).

### (R)-methyl 2-(3-(5-(2-oxopyridin-1(2H)-yl)pent-1-yn-1-yl)benzamido)propanoate VSN 82

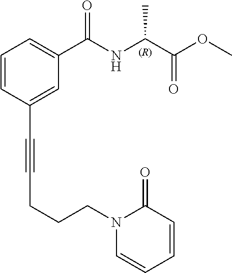

Following the general procedure described for amide coupling, the reaction of 3-(5-(2-oxopyridin-1(2H)-yl)pent-1-yn-1-yl)benzoic acid (0.60 g, 2.13 mmol), (R)-methyl 2-aminopropanoate.HCl (0.30 g, 2.13 mmol), DIPEA (0.37 ml, 2.13 mmol) and HATU (0.81 g, 2.13 mmol) in dry DCM (10 mL) after purification by chromatography (1-5% MeOH in DCM) gave (R)-methyl 2-(3-(5-(2-oxopyridin-1(2H)-yl)pent-1-yn-1-yl)benzamido)propanoate (0.43 g, 53.4% yield) as a viscous colourless oil.

(¹H) DMSO-d₆: 1.40 (3H, d, J=7.3 Hz), 1.93 (2H, qn, J=7.1 Hz), 2.44-2.50 (2H, m), 3.64 (3H, s), 3.97-4.05 (2H, m), 4.47 (1H, qn, J=7.2 Hz), 6.22 (1H, dt, J=1.4, 6.7 Hz), 6.36-6.40 (1H, m), 7.40 (1H, ddd, J=2.1, 6.6, 8.9 Hz), 7.46 (1H, t, J=7.8 Hz), 7.57 (1H, td, J=1.3, 7.7 Hz), 7.66-7.73 (1H, m), 7.82 (1H, td, J=1.5, 7.8 Hz), 7.92 (1H, t, J=1.5 Hz), 8.87 (1H, d, J=6.9 Hz) ppm.

δ(¹³C) DMSO-d₆: 16.05, 16.66, 27.51, 47.99, 48.29, 51.89, 80.35, 90.21, 105.23, 119.63, 123.10, 127.13, 128.68, 130.11, 133.90, 134.09, 139.14, 139.88, 161.44, 165.43, 173.05 ppm.

MS(ES+) m/z 367 (M+H).

### (R,Z)-methyl 2-(3-(5-(2-oxopyridin-1(2H)-yl)pent-1-en-1-yl)benzamido)propanoate VSN 87

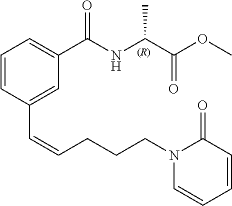

Following the general procedure for the Lindlar reduction, the hydrogenation of (R)-methyl 2-(3-(5-(2-oxopyridin-1(2H)-yl)pent-1-yn-1₁1)benzamido)propanoate (0.30 g, 0.82 mmol) gave the named product along with the fully saturated product (30%) (determined by ¹H NMR). Separation by column chromatography (EtOAc) and further purification by prep. HPLC (25% MeCN/water, acidic) gave the title compound (R,Z)-methyl 2-(3-(5-(2-oxopyridin-1(2H)-yl)pent-1-en-1-yl)benzamido)propanoate (87 mg, 28% yield) as a colourless gum. The other component was not isolated.

δ(¹H) DMSO-d₆: 1.41 (3H, d, J=7.3 Hz), 1.79 (2H, qn, J=7.5 Hz), 2.23-2.38 (2H, m), 3.65 (3H, s), 3.83-3.92 (2H, m), 4.49 (1H, qn, J=7.3 Hz), 5.77 (1H, td, J=7.3, 11.7 Hz), 6.14 (1H, dt, J=1.4, 6.7 Hz), 6.33-6.38 (1H, m), 6.51 (1H, br d, J=11.7 Hz), 7.38 (1H, ddd, J=2.1, 6.6, 8.9 Hz), 7.41-7.50 (2H, m), 7.62 (1H, dd, J=1.6, 6.8 Hz), 7.76 (2H, dd, J=1.9, 3.8 Hz), 8.84 (1H, d, J=6.9 Hz) ppm.

δ(¹³C) DMSO-d₆: 16.76, 25.23, 28.71, 48.16, 48.31, 51.94, 105.14, 119.57, 125.83, 127.67, 128.32, 128.69, 131.30, 132.46, 133.82, 136.97, 139.11, 139.84, 161.38, 166.20, 173.22 ppm.

MS(ES+) m/z 369 (M+H).

### R,Z)-2-(3-(5-(2-oxopyridin-1(2H)-yl)pent-1-en-1-yl)benzamido)propanoic acid VSN 88

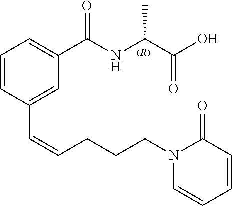

Following the general procedure for saponification, the reaction of (R,Z)-methyl 2-(3-(5-(2-oxopyridin-1(2H)-yl)pent-1-en-1-yl)benzamido)propanoate (40 mg, 0.11 mmol) with lithium hydroxide (5.2 mg, 0.22 mmol) gave (R,Z)-2-(3-(5-(2-oxopyridin-1(2H)-yl)pent-1-en-1-yl)benzamido)propanoic acid (30 mg, 76% yield) as a white solid.

δ(¹H) DMSO-d₆: 1.40 (3H, d, J=7.4 Hz), 1.78 (2H, qn, J=7.5 Hz), 2.25-2.36 (2H, m), 3.83-3.92 (2H, m), 4.42 (1H, qn, J=7.5 Hz), 5.76 (1H, td, J=7.3, 11.7 Hz), 6.13 (1H, dt, J=1.4, 6.7 Hz), 6.32-6.37 (I H, m), 6.50 (I H, br d, J=11.6 Hz), 7.37 (1H, ddd, J=2.1, 6.6, 9.0 Hz), 7.40-7.48 (2H, m), 7.60 (1H, dd, J=1.5, 6.8 Hz), 7.74-7.76 (2H, m), 8.67 (1H, d, J=7.3 Hz), 12.47 (1H, s) ppm.

δ(¹³C) DMSO-d₆: 16.89, 25.19, 28.66, 48.12, 48.17, 105.08, 119.53, 125.74, 127.60, 128.21, 128.69, 131.11, 132.35, 134.09, 136.89, 139.04, 139.76, 161.34, 166.03, 174.21 ppm.

MS(ES+) m/z 355 (M+H).

Synthesis of VSN 83, 89 and 90

### methyl 3-(5-(methylamino)pent-1-yn-1-yl)benzoate

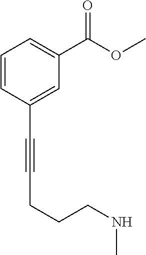 [SMILES: CNCCCC#Cc1cccc(C(=O)OC)c1]

To a solution of methyl 3-(5-((methylsulfonyl)oxy)pent-1-yn-1-yl)benzoate (2.40 g, 8.10 mmol) in dry THF (25.0 mL) was added methanamine (40 wt % in water) (7.01 ml, 81 mmol). The reaction was then stirred at 45° C. until judged complete by LCMS analysis. The reaction mixture was then partitioned between EtOAc (150 mL) and sat. aq. sodium bicarbonate (100 mL) and the aqueous layer was extracted with EtOAc (2×100 mL). The combined organic extracts were washed with water (3×100 mL) and brine (50 mL) then dried (MgSO₄), filtered and concentrated in vacuo to give the title product methyl 3-(5-(methylamino)pent-1-yn-1-yl)benzoate (2.0 g, 70.5% yield, 66% purity) as a 2:1 mixture with N-methyl-3-(5-(methylamino)pent-1-yn-1-yl)benzamide. No further purification attempted.

MS(ES+) m/z 232 (M+H).

### methyl 3-(5-(N-methylacetamido)pent-1-yn-1-yl)benzoate

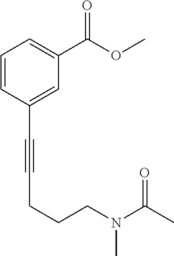 [SMILES: COC(=O)c1cccc(C#CCCCN(C)C(C)=O)c1]

A solution of methyl 3-(5-(methylamino)pent-1-yn-1-yl)benzoate (1.05 g, 4.54 mmol) (66% purity) and DIPEA (1.59 ml, 9.08 mmol) in dry DCM (10 mL) was treated with acetyl chloride (0.48 ml, 6.81 mmol). The reaction mixture was stirred at rt until judged complete by LCMS analysis. The volatiles were then removed in vacuo and the residue was partitioned between EtOAc (50 mL) and 1 N HCl (25 mL) and the aqueous layer was extracted with EtOAc (2×30 mL). The combined organic layers were washed with sat. aq. NaHCO₃ (25 mL), water (25 mL) and brine (25 mL) then dried (MgSO₄), filtered and concentrated in vacuo to give methyl 3-(5-(N-methylacetamido)pent-1-yn-1-yl)benzoate (1.20 g, 63.8% yield) as a 2:1 mixture with N-methyl-3-(5-(N-methylacetamido)pent-1-yn-1-yl)benzamide.

MS(ES+) m/z 274 (M+H).

### 3-(5-(N-methylacetamido)pent-1-yn-1-yl)benzoic acid

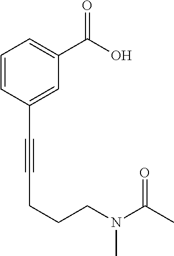 [SMILES: CC(=O)N(C)CCCC#Cc1cccc(C(=O)O)c1]

To a solution of methyl 3-(5-(N-methylacetamido)pent-1-yn-1-yl)benzoate (1.20 g, 4.39 mmol) in THF (15 mL) was added a solution of lithium hydroxide (0.210 g, 8.78 mmol) in water (3.0 mL). The reaction mixture was stirred at rt until judged complete by LCMS. The volatiles were removed in vacuo and the residue was partitioned between EtOAc (20 mL) and water (20 mL). The aqueous layer was extracted with with EtOAc (3×20 mL) then acidified to pH 1 with 1 N HCl and extracted with EtOAc (3×20 mL). The combined organic extracts were washed with water (20 mL) and brine (20 mL) then dried (MgSO₄), filtered and concentrated in vacuo to give 3-(5-(N-methylacetamido)pent-1-yn-1-yl)benzoic acid (0.68 g, 56.7% yield) as a viscous brown oil.

δ(¹H) DMSO-d₆: 1.67-1.87 (2H, m), 1.91 (1.5H, s), 1.98 (1.5H, s), 2.41 (1H, t, J=7.1 Hz), 2.45-2.49 (1H, m), 2.80 (1.5H, s), 2.97 (1.5H, s), 3.34-3.46 (2H, m), 7.44-7.53 (1H, m), 7.59-7.66 (1H, m), 7.85-7.94 (2H, m), 12.79 (1H, s) ppm (compound rotomeric hence some resonances are split).

MS(ES-F) m/z 260 (M+H).

### (R)-methyl 2-(3-(5-(N-methylacetamido)pent-1-yn-1-yl)benzamido)propanoate VSN 83

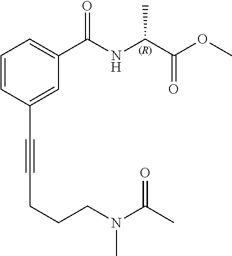

Following the general procedure described for amide coupling, the reaction of 3-(5-(N-methylacetamido)pent-1-yn-1-yl)benzoic acid (0.68 g, 2.49 mmol), (R)-methyl 2-aminopropanoate.HCl (0.35 g, 2.49 mmol), HATU (1.14 g, 2.99 mmol) and DIPEA (1.33 ml, 7.47 mmol) in dry DCM (10 mL) after purification by chromatography (1-3% MeOH in DCM) gave (R)-methyl 2-(3-(5-(N-methylacetamido)pent-1-yn-1-yl)benzamido)propanoate (0.40 g, 45.7% yield) as a pale yellow oil.

δ(¹H) DMSO-d₆ (@100° C.): 1.43 (3H, d, J=7.3 Hz), 1.77-1.87 (2H, m), 2.01 (3H, s), 2.43-2.50 (2H, m), 2.94 (3H, s), 3.39-3.48 (2H, m), 3.68 (3H, s), 4.54 (1H, qn, J=7.2 Hz), 7.43 (1H, t, J=7.7 Hz), 7.53 (1H, td, J=1.4, 7.7 Hz), 7.81 (1H, td, J=1.5, 7.8 Hz), 7.89 (1H, t, J=1.5 Hz), 8.50 (1H, br s) ppm.

δ(¹³C) DMSO-d₆: 15.90, 16.30, 16.66, 21.06, 21.72, 26.07, 26.74, 32.47, 35.81, 45.97, 48.28, 48.82, 51.89, 79.95, 80.31, 90.57, 90.98, 123.07, 123.23, 127.07, 127.13, 128.73, 128.76, 130.01, 130.06, 133.93, 133.95, 134.00, 165.42, 169.31, 169.66, 173.05 ppm. (Note: some peaks are doubled as compound is rotomeric).

MS(ES+) m/z 345 (M+H).

### (R,Z)-methyl 2-(3-(5-(N-methylacetamido)pent-1-en-1-yl)benzamido)propanoate VSN 89

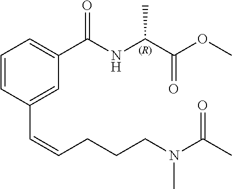

Following the general procedure for the Lindlar reduction, the hydrogenation of (R)-methyl 2-(3-(5-(N-methylacetamido)pent-1-yn-1-yl)benzamido)propanoate (0.32 g, 0.93 mmol) after separation by column chromatography (0-2% MeOH in DCM) and preparative HPLC (20-30% MeCN, acidic) gave the title compound (R,Z)-methyl 2-(3-(5-(N-methylacetamido)pent-1-en-1-yl)benzamido)propanoate (0.16 g, 48% yield) as a colourless oil.

δ(¹H) DMSO-d₆ (@100° C.): 1.44 (3H, d, J=7.3 Hz), 1.63-1.75 (2H, m), 1.95 (3H, s), 2.24-2.35 (2H, m), 2.95 (3H, s), 3.25-3.33 (2H, m), 3.68 (3H, s), 4.55 (1H, qn, J=7.2 Hz), 5.77 (1H, td, J=7.3, 11.7 Hz), 6.49 (1H, d, J=11.7 Hz), 7.41-7.49 (2H, m), 7.70-7.80 (2H, m), 8.42 (1H, d, J=5.9 Hz) ppm.

δ(¹³C) DMSO-d₆: 16.73, 21.04, 21.70, 25.34, 25.54, 26.97, 27.85, 32.53, 35.62, 46.17, 48.30, 49.40, 51.92, 125.73, 125.79, 127.58, 127.61, 128.31, 128.34, 128.60, 131.36, 131.42, 132.77, 133.04, 133.77, 133.79, 137.02, 137.09, 166.18, 166.22, 169.23, 169.50, 173.20 ppm (Note: some peaks are doubled as compound is rotomeric).

MS(ES+) m/z 347 (M+H).

### (R,Z)-2-(3-(5-(N-methylacetamido)pent-1-en-1-yl)benzamido)propanoic acid VSN 90

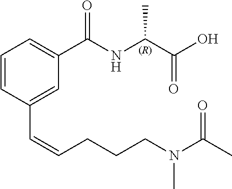

Following the general procedure for saponification, the reaction of (R,Z)-methyl 2-(3-(5-(N-methylacetamido)pent-1-en-1-yl)benzamido)propanoate (0.10 g, 0.289 mmol) with lithium hydroxide (14 mg, 0.58 mmol) gave (R,Z)-2-(3-(5-(N-methylacetamido)pent-1-en-1-yl)benzamido)propanoic acid (85 mg, 86% yield) as a colourless gum.

δ(¹H) DMSO-d₆ (@100° C.): 1.43 (3H, d, J=7.3 Hz), 1.67 (2H, m), 1.95 (3H, s), 2.24-2.35 (2H, m), 2.86 (3H, br s), 3.26-3.34 (2H, m), 4.49 (1H, qn, J=7.3 Hz), 5.77 (1H, td, J=7.3, 11.7 Hz), 6.49 (1H, br d, J=11.6 Hz), 7.40-7.48 (2H, m), 7.71-7.79 (2H, m), 8.26 (1H, m) ppm. Note: No OH observed.

δ(¹³C) DMSO-d₆: 16.88, 21.05, 21.71, 25.35, 25.54, 26.98, 27.85, 32.55, 35.64, 46.19, 48.17, 49.41, 125.71, 125.77, 127.57, 127.60, 128.26, 128.29, 128.34, 128.64, 131.23, 131.30, 132.71, 132.99, 134.04, 134.05, 136.97, 137.05, 166.07, 166.11, 169.23, 169.50, 174.24 ppm (Note: some peaks are doubled as compound is rotomeric). MS(ES+) m/z 333 (M+H).

Synthesis of VSN 84, 91 and 92

### methyl 3-(5-(N-methylmethylsulfonamido)pent-1-yn-1-yl)benzoate

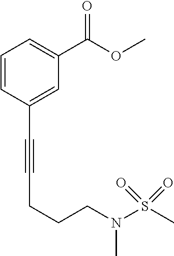 [SMILES: COC(=O)c1cccc(C#CCCCN(C)S(C)(=O)=O)c1]

A solution of methyl 3-(5-(methylamino)pent-1-yn-1-yl)benzoate (1.0 g, 4.32 mmol) (66% purity), DIPEA (1.13 ml, 6.49 mmol) in dry DCM (10 mL) was treated with methanesulfonyl chloride (0.42 ml, 5.40 mmol). The reaction mixture was stirred at rt until judged complete by LCMS analysis. The reaction mixture was partitioned between DCM (50 mL) and 1 N HCl (25 mL). The aq. layer was extracted with DCM (2×30 mL) befrore the combined organic extracts were washed with water (25 mL) and brine (25 mL) then dried (MgSO₄), filtered and concentrated in vacuo to give methyl 3-(5-(N-methylmethylsulfonamido)pent-1-yn-1-yl)benzoate (1.20 g, 59.2% yield) as a 2:1 mixture with N-methyl-3-(5-(N-methylmethylsulfonamido)pent-1-yn-1-yl)benzamide.

Carried forward as a mixture.

MS(ES+) m/z 310 (M+H).

### 3-(5-(N-methylmethylsulfonamido)pent-1-yn-1-yl)benzoic acid

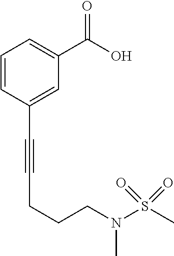 [SMILES: CN(CCCC#Cc1cccc(C(=O)O)c1)S(C)(=O)=O]

To a solution of methyl 3-(5-(N-methylmethylsulfonamido)pent-1-yn-1-yl)benzoate (1.20 g, 3.88 mmol) in THF (15 mL) was added a solution of lithium hydroxide (0.19 g, 7.76 mmol) in water (3.0 mL) The reaction mixture was stirred at rt until judged complete by LCMS. The volatiles were removed in vacuo and the residue was partitioned between EtOAc (20 mL) and water (20 mL). The aq. layer was acidified to pH 1 with 1 N HCl and extracted with EtOAc (3×30 mL). The combined organic extracts were washed with water (30 mL) and brine (30 mL) then dried (MgSO₄), filtered and concentrated in vacuo to give 3-(5-(N-methylmethylsulfonamido)pent-1-yn-1-yl)benzoic acid (0.50 g, 42.8% yield) as a pale brown solid.

δ(¹H) DMSO-d₆: 1.81 (2H, qn, J=7.1 Hz), 2.44-2.49 (2H, m), 2.77 (3H, s), 2.88 (3H, s), 3.14-3.22 (2H, m), 7.45-7.52 (1H, m), 7.63 (1H, td, J=1.4, 7.7 Hz), 7.85-7.93 (2H, m), 13.13 (1H, s) ppm

MS(ES+) m/z 296 (M+H).

### (R)-methyl 2-(3-(5-(N-methylmethylsulfonamido)pent-1-yn-1-yl)benzamido)propanoate VSN 84

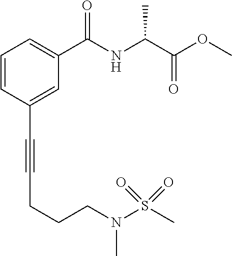 [SMILES: COC(=O)[C@@H](C)NC(=O)c1cccc(C#CCCCN(C)S(C)(=O)=O)c1]

Following the general procedure described for amide coupling, the reaction of 3-(5-(N-methylmethylsulfonamido)pent-1-yn-1-yl)benzoic acid (0.50 g, 1.69 mmol), (R)-methyl 2-aminopropanoate.HCl (0.26 g, 1.86 mmol), HATU (0.74 g, 1.95 mmol) and DIPEA (0.739 ml, 4.23 mmol) in dry DCM (10 mL) after purification by chromatography (30-70% EtOAc in iso-hexanes) gave (R)-methyl 2-(3-(5-(N-methylmethylsulfonamido)pent-1-yn-1-yl)benzamido)propanoate (0.39 g, 57.5% yield) as a pale yellow oil.

δ(¹H) DMSO-d₆: 1.40 (3H, d, J=7.3 Hz), 1.82 (2H, qn, J=7.1 Hz), 2.46-2.49 (2H, m), 2.77 (3H, s), 2.88 (3H, s), 3.18 (2H, t, J=7.0 Hz), 3.64 (3H, s), 4.47 (1H, qn, J=7.1 Hz), 7.46 (1H, t, J=7.8 Hz), 7.56 (1H, td, J=1.3, 7.7 Hz), 7.82 (1H, td, J=1.4, 7.8 Hz), 7.91 (1H, t, J=1.5 Hz), 8.86 (1H, d, J=6.9 Hz) ppm.

δ(¹³C) DMSO-d₆: 15.92, 16.66, 26.59, 34.54, 34.57, 48.28, 48.60, 51.90, 80.18, 90.62, 123.17, 127.09, 128.73, 130.04, 133.93, 134.04, 165.43, 173.05 ppm.

MS(ES+) m/z 381 (M+H).

### (R,Z)-methyl 2-(3-(5-(N-methylmethylsulfonamido)pent-1-en-1-yl)benzamido)propanoate VSN 91

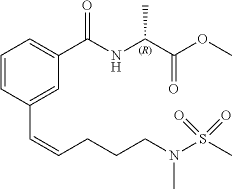

Following the general procedure for the Lindlar reduction, the hydrogenation of (R)-methyl 2-(3-(5-(N-methylmethylsulfonamido)pent-1-yn-1-yl)benzamido)propanoate (0.32 g, 0.84 mmol) gave the named product along with the trans double bond isomer (<5%) and fully saturated product (10%) (determined by ¹H NMR). Separation by column chromatography (10% EtOAc in DCM) gave the title compound (R,Z)-methyl 2-(3-(5-(N-methylmethylsulfonamido)pent-1-en-1-yl)benzamido)propanoate (0.19 g, 59% yield) as a colourless oil. The other 2 components were not isolated.

δ(¹H) DMSO-d₆: 1.40 (3H, d, J=7.3 Hz), 1.68 (2H, qn, J=7.1 Hz), 2.26-2.35 (2H, m), 2.72 (3H, s), 2.83 (3H, s), 3.03-3.07 (2H, m), 3.64 (3H, s), 4.48 (1H, qn, J=7.2 Hz), 5.77 (1H, td, J=7.3, 11.8 Hz), 6.50 (1H, td, J=1.8, 11.8 Hz), 7.43 7.51 (2H, m), 7.73-7.80 (2H, m), 8.80 (1H, d, J=6.9 Hz) ppm. Note: contaminated with 5-10% trans isomer.

δ(¹³C) DMSO-d₆: 16.71, 25.23, 27.48, 34.41, 34.48,48.26, 49.04, 51.87, 125.72, 127.60, 128.27, 128.52, 131.29, 132.66, 133.79, 137.02, 166.16, 173.14.ppm.

MS(ES+) m/z 383 (M+H).

### (R,Z)-2-(3-(5-(N-methylmethylsulfonamido)pent-1-en-1-yl)benzamido)propanoic acid VSN 92

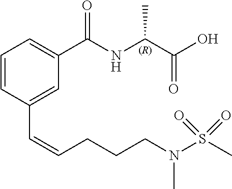

Following the general procedure for saponification, the reaction of (R,Z)-methyl 2-(3-(5-(N-methylmethylsulfonamido)pent-1-en-1-yl)benzamido)propanoate (0.11 g, 0.288 mmol) with lithium hydroxide (14 mg, 0.58 mmol) gave (R,Z)-2-(3-(5-(N-methylmethylsulfonamido)pent-1-en-1-yl)benzamido)propanoic acid (0.1 g, 90% yield) as a white solid. Note: 5% isomerisation to trans double bond under reaction conditions observed.

δ(¹H) DMSO-d₆: 1.43 (3H, d, J=7.3 Hz), 1.72 (2H, qn, J=7.4 Hz), 2.33 (2H, dq, J=1.8, 7.5 Hz), 2.75 (3H, s), 2.81 (3H, s), 3.08-3.14 (2H, m), 4.49 (1H, qn, J=7.3 Hz), 5.78 (1H, td, J=7.3, 11.7 Hz), 6.50 (1H, dd, J=11.7, 1.6 Hz), 7.42-7.47 (2H, m), 7.72-7.78 (2H, m), 8.26 (1H, d, J=6.9 Hz), 12.04 (1H, br s) ppm.

δ(¹³C) DMSO-d₆: 16.88, 25.27, 27.52, 34.46, 34.48, 48.17, 49.07, 125.74, 127.63, 128.27, 128.59, 131.21, 132.65, 134.05, 137.00, 166.08, 174.24 ppm.

MS(ES+) m/z 369 (M+H).

Methods

Patch Clamp Studies

Cell Culture

The human umbilical vein derived endothelial cell line, EA.hy926 (Edgell et al., Proc Natl Acad Sci USA. 1983 June; 80(12): 3734-7) at passage >45 was grown in DMEM containing 10% FCS and 1% HAT (5 mM hypoxanthine, 20 μM aminopterin, 0.8 mM thymidine) and cells were maintained in an incubator at 37° C. in 5% CO₂ atmosphere. Cells were plated on either 10 mm (for patch-clamp recordings) or 30 mm glass cover slips (for Ca²⁺ measurements).

Electrophysiological Recordings

Membrane potential of EA.hy926 cells was recorded using the perforated patch clamp technique as described previously (Bondarenko et al, 2010, Br. J. Pharmacol. 161, 308-320). For membrane potential recordings from EA.hy926 cells the standard bath solution contained (in mM): 140 NaCl, 5 KCl, 1.2 MgCl₂, 10 HEPES, 10 glucose, 2.4 CaCl₂, patch pipettes were filled with a solution containing (in mmol/L): 140 KCl; 0.2 EGTA; 10 HEPES (pH adjusted to 7.2 using KOH). The resistance of the pipettes was 3-5 MΩ for whole cell and 6-8 MΩ for single channel recordings.

Single-channel recordings were obtained from excised inside-out membrane patches in symmetrical solutions. The pipettes were filled with (in mM) 140 KCl, 10 HEPES, 1 MgCl₂, 5 EGTA, 4,931 CaCl₂ with pH 7.2 by adding KOH (i.e. 10 μM free Ca²⁺, G. Droogmans, Leuven, Belgium; ftp://ftp.cc.kuleuven.ac.be/pub/droodmans/cabuf.zip). Cells were perfused with a standard bath solution containing (in mM) 140 NaCl, 5 KCl, 1.2 MgCl₂, 10 HEPES, 10 glucose, 2.4 CaCl₂. Following gigaseal formation, bath solution was switched to the following (in mM) 140 KCl, 10 HEPES, 1 MgCl₂, 5 EGTA and a desired free Ca²⁺ concentration which was adjusted by adding different amounts of CaCl₂ calculated by the program CaBuf. pH was adjusted to 7.2 by adding KOH. Membrane currents and potential were recorded using a List EPC7 amplifier (List, Germany) and pClamp (version 8.2, Axon Instruments) software.

Vasodilation in Rat Mesenteric Arteries

Rats were stunned by a blow to the back of their neck and killed by cervical dislocation in compliance with schedule 1 of the UK Animals (Scientific Procedures) Act 1986. The third-order branches of the superior mesenteric artery, which provides blood supply to the intestine, were then removed and cleaned of adherent tissue.

Segments (2 mm in length) were mounted in a Mulvany-Halpern type wire myograph (Model 610M; Danish Myo Technology, Aarhus, Denmark) and maintained at 37° C. in gassed (95% O₂/5% CO₂) Krebs-Henseleit solution of the following composition (mM): NaCl 118, KCl 4.7, MgSO₄ 1.2, KH₂PO4 1.2, NaHCO, 25, CaCl₂ 2, D-glucose 10 as previously described (Ho and Randall, 2007). Arteries were equilibrated and set to a basal tension of 2-2.5 mN. The integrity of the endothelium was assessed by precontracting the vessel with 10 μM methoxamine (an α₁-adrenoceptor agonist), followed by relaxation with 10 μM carbachol (a muscarinic acetylcholine receptor agonist); vessels showing relaxations of greater than 90% were designated as endothelium-intact. When endothelium was not required, it was removed by rubbing the intima with a human hair; carbachol-induced relaxation of less than 10% indicated successful removal. After the test for endothelial integrity, arteries were left for 30 min and then precontracted with 10 μM methoxamine (or 60 mM KCl), this was followed by construction of a cumulative concentration-relaxation curve to VSN16R (10 nM-1 μM). Most experiments were performed in matched vessels; effects of putative modulators or endothelial removal were compared with the control responses obtained in separate vessels of the same rat. Potential modulators were added to the myograph bath 30 min before measurement, and kept present during, construction of the concentration-relaxation curve.

VSN16R Reduces Intraocular Pressure (IOP)

The effect of VSN16R on IOP was measured using the techniques described in the literature (Guo L. et aI, Investigative Ophthalmology & Visual Science, January 2005, Vol 46, No. 1 p 175-182; Guo L. et al, Investigative Ophthalmology & Visual Science, February 2006, Vol 47, No. 2 p 626-633; Cordeiro F. et al, PNAS, Aug. 14, 2007; Vol 104; No. 33, p 13444-13449; Cordeiro F. et al, PNAS, Sep. 7, 2004; Vol 101; No. 36, p 13352-13356).

The results show that VSN16R significantly reduces IOP at 0.5 h (mean 9.82) (p<0.05) but not 1 h (10.79), compared to BL (11.18), suggesting VSN16R has a very short half-life and repeated administration may be necessary to maintain lowering IOP.

Identification of a Subunit Isoforms and β Subunits in EA.hy926 Cells Using RNA Sequencing

RNA sequencing (RNAseq) is a suitable experimental approach for identifying the different α subunit isoforms present in EA.hy926 cells, leading to direct and unbiased ‘reading’ of the different mRNA transcripts expressed in the cells. Simultaneously, the expression of the four β subunits is quantified.

EA.hy926 cells were obtained from LGC Standards (ATCC-CRL-2922) and cultured in Dulbeco's modified Eagle's media supplemented with 10% foetal bovine serum. To collect total RNA, cell culture media was removed from the cells and following a wash with phosphate buffered saline cells were disrupted with TRIzol® Reagent by repeated pipetting. Following 5 min incubation at room temperature, chloroform was added to the samples, which were shaken, left to rest and then centrifuged at 12000 g for 15 minutes. The resulting upper aqueous phase was washed with 70% ethanol, mixed well and loaded on an RNeasy column. Thereafter the Qiagen RNeasy® Mini Kit protocol was followed to extract and purify mRNA. mRNA integrity was assessed by microfluidic capillary electrophoresis using the Agilent 2100 Bioanalyzer. RNA sequencing was performed at the UCL Genomics facility (UCL Institute of Child Health) using the Illumina NextSeq 500 platform.

The FASTQ files generated were aligned to the UCSC Homo sapiens hg19 reference genome using the TopHat2 software (Illumina). FPKM (Fragments Per Kilobase of exon per Million reads) values for all genes and transcripts were obtained.

Results

The β subunit isoforms expressed in the cells are predominantly the β4 subunit and very low levels of the β3 subunit (Table 1).

The α subunit isoforms expressed in the cells are isoforms with no insert in the C2 region (ZERO transcripts), as well as three different two-exon short and possibly non-functioning transcripts, which would only form the extracellular N-terminal region (Table 2).

From the above data, it can be concluded that that EA.hy926 cells predominantly express β4-ZERO and to a much much lesser extent β3-ZERO BK channels. This significantly narrows down the possibilities about where VSN16R may act. With regard to the β3 subunit, this is a developmentally expressed inhibitory subunit that is expressed in adult testis.

Conclusions

Taking together the mouse sequencing data (low β2 and high β4 subunit expression) and the EA.hy926 data (low β3 and high β4 subunit expression), VSN16R is likely to interact with a channel comprising of the β4 subunit.

Taking the data on the recombinant ZERO isoform alone into consideration, this strongly suggests that the β4-ZERO combination is the only candidate BK channel on which VSN16R acts. β4-ZERO is regarded as the neuronal BK type.

VSNT6R Does not Act on BKCa Channels Formed by the Exon-Less Alpha Subunit

Studies by the Applicant have demonstrated that VSN16R is a selective BKCa opener that does not act on BKCa channels formed by the exon-less alpha subunit, in the absence of any beta or gamma subunits, expressed in HEK293 cells.

The results of these experiments are illustrated in FIGS. 11A-D discussed in more detail below.

More specifically, FIG. 11A shows a representative time-course of action of VSN16R (20 μM) on BKCa currents measured in the whole-cell configuration and elicited by 200 ms-long voltage steps from −40 mV to +70 mV in the presence of 200 nM calcium. VSN16R was applied for ˜10 min and did not display any enhancing effect on the current, which was suppressed by application of the BKCa inhibitor paxilline (10 μM).

FIG. 11B shows current-voltage relationships for BKCa currents measured under control conditions, in the presence of the BKCa opener VSN16R (20 μM), and in the presence of paxilline (10 μM). VSN16R did not affect the BKCa current at any voltage and did not shift the voltage-depence of activation of BKCa channels.

FIG. 11C shows relative enhancement of BKCa currents caused by VSN16R (red symbols; 20 μM) or by the non-selective BKCa opener NS19504 (10 μM). While VSN16R did not enhance BKCa currents in 7 cells tested, NS19504 approximately doubled the BKCa current elicited in response to voltage steps from −40 mV to +70 mV in the presence of 200 nM calcium. Paxilline (10 μM) or TEA (5 mM) consistently suppressed the BKCa current after application of the openers.

FIG. 11D shows that VSN16R (20 μM) did not affect the activation voltage of BKCa current when applied in the presence of various concentrations of intracellular calcium (1 μM; 200 nM; nominally 0 M).

These experiments confirm that VSN16R does not act on the alpha subunit of the BKCa channels, thereby complementing the conclusions from the RNA sequencing experiments on EA.hy926 cells.

Effect of VSN16R in Fragile X Syndrome

Animals: C57BL/6.J. Fmr1-K02 mice, which have a deletion of the promoter and exonl of the Fmr1 gene (Mientjes et al., 2006) and C57BL/6J wild type (WT) were originated from the Jackson laboratory (Ann Harbor, USA). Mice were housed in groups (4-6 per cage) and all animals were provided with ad libitum food and water unless otherwise stated. Mice were maintained on a 12 h light/12 h dark cycle (lights off 19:00 to 7:00) in a temperature-controlled environment (21±1° C.). Testing was conducted in the light phase. Mice were housed in commercial cages and experiments were performed in line with the United Kingdom Animals (Scientific procedures) Act 1986. All experiments were conducted with experimentors blind to genotype and drug treatment. Each experimental group contained 10 animals.

Drug Treatment: VSN16R was dissolved in saline for injection intravenously via a tail vein using 2 mg/kg a 30 g needle in a volume of 0.1 ml. Animals were inspected for differences in coat appearance, to detect whether any piloerection was present. The eye condition (runny eyes or porphyria, ptosis), gait appearance, tremor, tail tone, reactivity to handling was assessed to detect adverse behavioural effect.

Open field: The open field apparatus was used to test multiple processes including anxiety/hyperactivity and habituation to a novel environment, in which decreased exploration as a function of repeated exposure to the same environment is taken as an index of memory. This was studied in two sessions of exposure to the open field, occurring at 10 minutes and 24 hours after exposure to the environment. The apparatus was a grey PVC enclosed arena 50×30 cm divided into 10 cm squares. Mice were brought to the experimental room 5-20 min before testing. A mouse was placed into a corner square facing the corner and observed for 3 min. The number of squares entered (whole body) (locomotor activity) and rears (both front paws off the ground, but not as part of grooming) were counted. The movement of the mouse around the field was recorded with a video-tracking device for 3 min (vNT4.0, Viewpoint). VSN16R was once/once daily for 14 days and 30 minutes prior to baseline analysis. The test was then performed 10min and 24 hour after the test and the drug was not administered after the initial injection prior to the baseline assessment.

Contextual fear conditioning: Animals are trained to expect an electroshock treatment within a defined environment, such that on subsequent presentation of the environment, freezing behaviours are induced. Testing involved placing the animal in a novel environment (dark chamber), providing an aversive stimulus (a 1-sec electric shock, 0.2 mA, to the paws), and then removing it. The conditioning chamber used was from Kinder Scientific, USA. VSN16R was injected following baseline analysis. VSN16R was once/once daily for 14 days prior to assessment and animals were tested 30 mins following delivery of VSN16R.

Marble burying: Mice will dig as part of their typical behaviours and this was detected using transparent plastic cages were filled with a 10 cm deep layer of sawdust on top of which 10 glass marbles were placed in two rows. Each animal was left undisturbed in such a cage for 30 min, after which the number of marbles that were buried to at least ⅔ of their depth was recorded. The number of marbles was assessed 30 mins following delivery of VSN16R. VSN16R was once/once daily for 14 days prior to assessment and animals were tested 30 mins following delivery of VSN16R.

Statistics: Experimental groups contained 10 animals per group and results are reported as group means±standard deviations. Differences between groups were assessed using t tests analysis of variance tests using Sigmaplot Software. All experimenters were fully blinded to treatment conditions during the collection, assembly, and interpretation of the data.

Results

Following the injection of 2 mg/kg VSN16R i.v. over a period of 2 weeks it was found to be well tolerated and no instances of toxicity were observed. When C57BL/6 wildtype mice were introduced into an open field chamber there was exploratory behaviour. As anticipated Fmr1-deficient mice exhibited a significantly elevate amount of exploratory behaviour (FIG. 12A). This was inhibited following treatment of 2 mg/kg i.v. VSN16R such that movement behaviours were comparable to that observed in wildtype mice (FIG. 12A). Furthermore as anticipated wildtype C57BL/6 mice demonstrated evidence of memory of the environment and moved around less compared the initial presentation of the open field (FIG. 12A versus FIG. 12B & FIG. 12C). In all instances Fmr1-deficient mice exhibited significant (P<0.001) hyperactivity compared VSN16R-treated knockout mice, whereas as VSN16R-treated wildtype mice exhibit comparable movement behaviour to vehicle-treated wildtype mice. This normalization of neurological behaviour of Fmr1-deficient mice suggested that both short-term and long-term memory deficits in Fmr1 mice were inhibited. Additional neurological behaviours are different in Fmr1 mice (Deacon et al. 2015) and included exaggerated (P<0.001) fear conditioning (FIG. 13) and normal digging behaviours (FIG. 14). In comparison to the activity observed in Fmr1 knockout mic, VSN16R significantly (P<0.001) limited the exaggerated behaviour compared to vehicle treated Fmr-1 knockout mice. However at the 2 mg/kg dose tested the levels of activity were not normalised to those found in wildtype mice, where VSN16 exhibited no-inhibitory effect compared to vehicle treated wildtype mice.

Discussion

This study demonstrates that VSN16R can significantly attenuate all behaviours tested that are exaggerated in Fmr1-deficient mice and suggests that VSN16R may have some utility in the treatment of symptoms of Fragile X/Autism. Efficacy was consistent with that seen in treatment with high doses of other agents (Deacon et al. 2015) and the drug was found to be well tolerated. VSN16R was shown to normalise the memory and exploratory behaviours to the level of wildtype mice, but could not normalise all behaviours to the level of wildtype animals, which is also consistent with other studies (Deacon et al. 2015). However, it is possible that better effect could have been achieved through dosing of higher levels, as 2 mg/kg was at the low end of known is efficacy (≥1 mg/kg p.o.) and much higher doses (daily ≥1000 mg/kg p.o.) are tolerable, and through the use of the oral route that has a 10 times longer half-life than the intravenous route (t½=6 minutes). It is also possible that greater CNS penetration of the compound could augment efficacy, however that efficacy was detected suggests that sufficient compound can enter the central nervous system to inhibit the neurological behaviours caused by the Fmr1 loss.

The data support the view that BKCa modulators can inhibit the signs of disease associated with fragile X (Hébert et al. 2014). In the Fmr1 knockout mice deficits in KNCMA expression was noted suggesting that there is a loss-of-function influence that drives the neurological symptoms. It is evident from studies in epilepsy that both loss-of-function and gain of function of BKCa activity can both result in neural hyperexcitability depending on the neural circuitary affected (N'Gouemo 2014). VSN16R can result in hyperpolarization of neural membranes to limit neural excitability as can occur with modulators of the KCNMA1 pore of the BKCa channel (Laumonnier et al. 2006). These finding suggest that VSN16R and related compounds have the ability to limit symptoms of fragile X.

Pig Aorta

Studies by the applicant have demonstrated that VSN16 does not stimulate pig aortic endothelial cells. By way of illustration, FIG. 2A shows the time course of the current development at −100 mV (lower) and +85 mV (upper) in primary pig aorta endothelia, which do not express BK_{Ca} KCNMB subunits, in response to VSN16R or the removal of potassium. Pig aortic endothelial cells only express KNCMAI and not KNCMB1-4 (Papassotiriou et al). Thus, the absence of stimulation indicates a lack of effect on KNCMA1.

Endothelial Cell Dependence

Studies were carried out to determine the effect of VSN16 on third order rat mesenteric arteries (Parmar et al). FIG. 15 shows the effect of VSN16 (percent relaxation versus log [VSN16]) on rat mesenteric arteries: (A) endothelial intact (n=11), endothelium denuded (n=11) cultures or endothelium intact cultures in the presence of 60 mM KCl (n=6); (B) vehicle-treated controls (n=17 animals) or pretreated with 50 nM apamin (n=7), 50 nM iberotoxin (n=5), 50 nM charybdotoxin (n=6) or a combination of apamin and charybdotoxin (n=6).

The results indicated that VSN16R can induce vasorelaxation in an endothelium-dependent (P<0.001) manner (FIG. 15A) and is inhibited by antagonists of BK_{Ca} channels, notably by Iberotoxin (P<0.05) and charybdotoxin (P<0.01. FIG. 15B). In addition, the relaxation is dependent of potassium flux, as VSN16R produced no relaxation in the presence of extracellular 60 mM KCl, supporting an action via BK_{Ca} channels (FIG. 15A).

This endothelial dependence indicates that VSN16R does not act via the alpha (KCNMA1) pore or the beta 1(KCNMB1) pore as it would relax the endothelial cell independent activity. If VSN16R targeted the alpha pore (KCNMA1) directly, one would expect to see an effect in endothelium-denuded mesenteric arteries where it would bind to the smooth muscle known to express the alpha (KCNMA1) and the betal (KCNMB1) chain. The results therefore indicate that the action is not via smooth muscle KCNMA1, KCNMB1.

Vas Deferens

Mouse vas deferens were treated with either DMSO vehicle or 100 nM VSN16R 30 min before the first organ bath injection of various concentrations of βγ-methylene ATP into the organ bath (FIG. 16). The results represent the mean±SEM of βγ-methylene ATP-induced increases in tension (expressed in grams) of electrically unstimulated vasa deferentia. (n=6/group). Vehicle EC₅₀=1347 nM, Vehicle VSN16R=1832 nM (95% Cl 836-40 11 nM). The results indicate that VSN16R does not affect inhibited beta gamma methylene adenosine triphosphatase-induced contraction in electrically unstimulated vasa deferentia, indicating that the action of VSN16R is not directly on smooth muscle.

By way of summary, experiments by the applicant have demonstrated that VSN16 does not act via the smooth muscle BK_{Ca} isoform as shown here by the endothelial cell dependence in induced arterial relaxation, and the lack of relaxing effect of VSN16R on βγ-methylene ATP on smooth muscle.

The studies described herein demonstrate that VSN16R and its analogues thereof are novel BK channel activators with potential for the treatment of several diseases. These can be characterised as those in which BK channel activity is dysfunctional as a result of disease pathology or a genetic condition. In addition the effect of BK channel activation is to reduce excitability in cells particularly in neuronal cells and thus VSN16R will be useful to treat diseases in which cells, particularly neuronal cells have become hyperexcitable. Such conditions include those mentioned above.

Various modifications and variations of the described methods of the invention will be apparent to those skilled in the art without departing from the scope and spirit of the invention. Although the invention has been described in connection with specific preferred embodiments, various modifications of the described modes for carrying out the invention which are obvious to those skilled in chemistry or related fields are intended to be within the scope of the following claims.

### REFERENCES

Bassell G J, Warren S T (2008). Fragile X syndrome: loss of local mRNA regulation alters synaptic development and function. Neuron 60: 201-214.

Bhakar A L, Dölen G, Bear M F (2012). The pathophysiology of fragile X (and what It teaches us about synapses). Annu Rev Neurosci 35: 417-443.

Briault S, Perche O (2012). Compositions for the treatment of fragile X syndrome WO2013001412.

Darnell J C, Van Driesche S J, Zhang C, Hung K Y, Mele A, Fraser C E et al. (2011). FMRP stalls ribosomal translocation on mRNAs linked to synaptic function and autism. Cell 146: 247-261.

Deacon R M (2006) Housing, husbandry and handling of rodents for behavioral experiments. Nat protocols 1(2):936-46.

Deacon R M, Glass L, Snape M, Hurley M J, Altimiras F J, Biekofsky R R, et al. (2015) NNZ-2566, a novel analog of (1-3)IGF-1, as a potential therapeutic agent for fragile X syndrome. Neuromolecular Med 17:71-82.

Dolan B M, Duron S G, Campbell D A, Vollrath B, Shankaranarayana Rao B S, Ko H Y, Lin G G, Govindarajan A, Choi S Y, Tonegawa S (2013) Rescue of fragile X syndrome phenotypes in Fmr1 KO mice by the small-molecule PAK inhibitor FRAX486. Proc Natl Aced Sci USA 110:5671-5676.

Gessner G, Cui Y M, Otani Y, Ohwada T, Soom M, Hoshi T, Heinemann S H. (2012). Molecular mechanism of pharmacological activation of BK channels. Proc Natl Acad Sci USA.;109:3552-3557.

Grossman A W, Aldridge G M, Weiler I J, Greenough W T (2006). Local protein synthesis and spine morphogenesis: fragile X syndrome and beyond. J Neurosci 26: 7151-7155.

Hagerman (1997) Fragile X syndrome. Molecular and clinical insights and treatment issues. West J Med 166(2):129-37.

Hébert B, Pietropaolo S, Même S, Laudier B, Laugeray A, Doisne N, Quartier A, Lefeuvre S, Got L, Cahard D, Laumonnier F, Crusio W E, Pichon J, Menuet A, Perche O, Briault S. (2014) Rescue of fragile X syndrome phenotypes in Fmr1 KO mice by a

BKCa channel opener molecule. Orphanet J Rare Dis. 2014; 9:124.

Laumonnier F, Roger S, Guérin P, Molinari F, M'rad R, Cahard D, Belhadj A, Halayem M, Persico A M, Elia M, Romano V, Holbert S, Andres C, Chaabouni H, Colleaux L, Constant J, Le Guennec J Y, Briault S. Association of a functional deficit of the BKCa channel, a synaptic regulator of neuronal excitability, with autism and mental retardation. Am J Psychiatry. 2006;163:1622-1629.

Levenga, J., Hayashi, S., de Vrij, F. M., Koekkoek, S. K., van der Linde, H. C., Nieuwenhuizen, I., Song, C., Buijsen, R. A., Pop, A. S., Gomezmancilla, B., Nelson, D. L., Willemsen, R., Gasparini, F., and Oostra, B. A. (2011). AFQ056, a new mGluR5 antagonist for treatment of fragile X syndrome. Neurobiol. Dis. 42, 311-317.

Michalon A, Sidorov M, Ballard T M, Ozmen L, Spooren W, Wettstein J G, Jaeschke G, Bear M F, Lindemann L (2012) Chronic Pharmacological mGlu5 Inhibition Corrects Fragile X in Adult Mice. Neuron 74:49-56.

Mientjes E J, Nieuwenhuizen I, Kirkpatrick L, Zu T, Hoogeveen-Westerveld M, Severijnen L, Rife M, Willemsen R, Nelson D L, Oostra B A (2006). The generation of a conditional Fmrl knock out mouse model to study Fmrp function in vivo. Neurobiology of disease 21: 549-555.

Oostra B A, Willemsen R. (2003) A fragile balance: FMR1 expression levels. Hum Mol Genet. 12 Spec No 2:R249-57.

Papassotiriou J, Köhler R, Prenen J, Krause H, Akbar M, Eggermont J, Paul M, Distler A, Nilius B, Hoyer J. Endothelial K(+) channel lacks the Ca(2+) sensitivity-regulating beta subunit. FASEB J. 2000; 14:885-894.

Parmar N, Ho W S (2010). N-arachidonoyl glycine, an endogenous lipid that acts as a vasorelaxant via nitric oxide and large conductance calcium-activated potassium channels. Br J Pharmacol 2010; 160: 594-603

What is claimed is:

1. A method of treating a disorder associated with BK channel modulation in a subject in need thereof, comprising administering to the subject a compound of formula I, or a pharmaceutically acceptable salt, solvate or prodrug thereof, 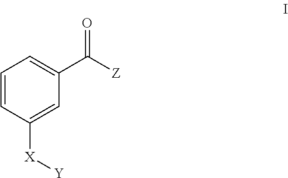
wherein:
Z is OR¹⁶ or NR¹⁷R¹⁸;
R¹⁶ is H or alkyl;
R¹⁷ is H or alkyl;
R¹⁸ is alkyl or cycloalkyl, each of which is optionally substituted by one or more substituents selected from OH, halogen and COOR¹¹;
X is a group selected from
—C≡C—(CH₂)ₚ—;
—C(R⁵)═C(R⁶)—(CH₂)_{q}—; and
—C(R⁵)(R⁶)C(R⁷)(R⁸)—(CH₂)ᵣ—;
where each of R⁵, R⁶, R⁷ and R⁸ is independently H or alkyl, and each of p, q and r is independently 1, 2, 3, 4 or 5;
Y is a group selected from:
CN;
COOR²;
CONR³R⁴;
SO₂NR³R¹⁰;
NR¹²COR¹³;
NR¹⁴SO₂R¹⁵; and
a heterocyclic group selected from oxadiazolyl, thiazolyl, iso-thiazolyl, oxazolyl, iso-oxazolyl, pyrazolyl and imidazolyl;
where each of R², R³ and R⁴ is independently H or alkyl; or R³ and R⁴ are linked, together with the nitrogen to which they are attached, to form a 5 or 6-membered heterocycloalkyl or heterocycloalkenyl group, said heterocycloalkyl or heterocycloalkenyl group optionally containing one or more further groups selected from O, N, CO and S, and where each of R⁹, R¹⁰, R¹¹, R¹², R¹³, R¹⁴ and R¹⁵ is independently H or alkyl.

2. The method according to claim 1 wherein Z is NR¹⁷R¹⁸.

3. The method according to claim 1 wherein R¹⁷ is H and R¹⁸ is selected from alkyl and cycloalkyl, each of which is optionally substituted by one or more substituents selected from OH and F.

4. The method according to claim 1 wherein the method comprises administering to the subject a compound comprising formula IA, or a pharmaceutically acceptable salt, solvate or prodrug thereof, 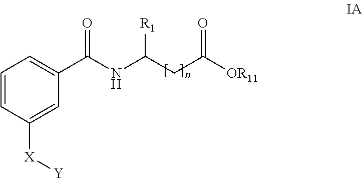
wherein:
X, Y and R¹¹ are as defined in claim 1;
n is 0 or 1; R¹ is selected from H, alkyl and aralkyl, wherein said alkyl and aralkyl groups may be optionally substituted by one or more OH groups;

5. The method according to claim 4 wherein R¹ is selected from H, Me, Et, ⁿPr, ⁱPr, CH₂-phenyl, CH₂-[4-(OH)-phenyl], CH₂OH, CH(OH)CH₃, CH(CH₃)CH₂CH₃ and CH₂CH(CH₃)₂.

6. The method according to claim 1 wherein Y is selected from CN, CON(Me)₂, CONHMe, CONHEt, SO₂N(Me)₂, N(Me)COMe, N(Me)SO₂Me, CO-piperidinyl, CO-pyrrolidinyl, oxadiazolyl and thiazolyl, more preferably, CON(Me)₂.

7. The method according to claim 1 wherein X— is cis —C(R⁵)═C(R⁶)—(CH₂)_{q}— and q is 2, 3 or 4.

8. The method according to claim 7 wherein X is —CH═CH—(CH₂)_{q}— and q is 2 or 3.

9. The method according to claim 1 wherein X is —C(R⁵)(R⁶)C(R⁷)(R⁸)—(CH₂)ᵣ— and r is 2, 3 or 4.

10. The method according to claim 9 wherein X is —CH₂—CH₂—(CH₂)ᵣ— and r is 2 or 3.

11. The method according to claim 1 wherein X is —C≡C—(CH₂)ₚ—, where p is 1, 2, 3, 4, or 5.

12. The method according to claim 4 which is of formula Ia, or a pharmaceutically acceptable salt, solvate or prodrug thereof,

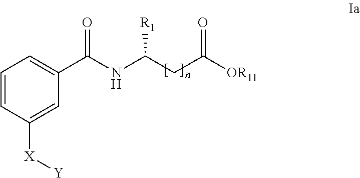

13. The method according to claim 4 which is of formula Ib, or a pharmaceutically acceptable salt, solvate or prodrug thereof,

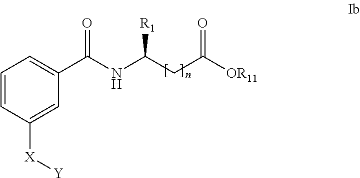

14. The method according to claim 4 wherein n is 0.

15. The method according to claim 4 wherein R¹ is Me, CH₂Ph or CH₂OH.

16. The method according to claim 4 wherein n is 0, R¹ is Me and X is —CH≡CH—(CH₂)₃— or —CH₂—CH₂—(CH₂)₃—.

17. The method according to claim 4 wherein n is 1 and R¹ is H.

18. The method according to claim 1 comprising administering to the subject a compound selected from the following: 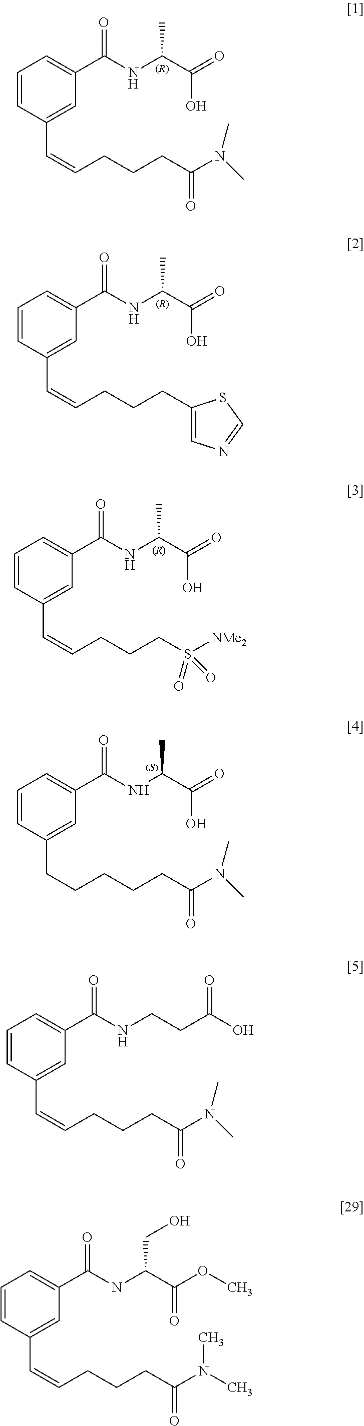 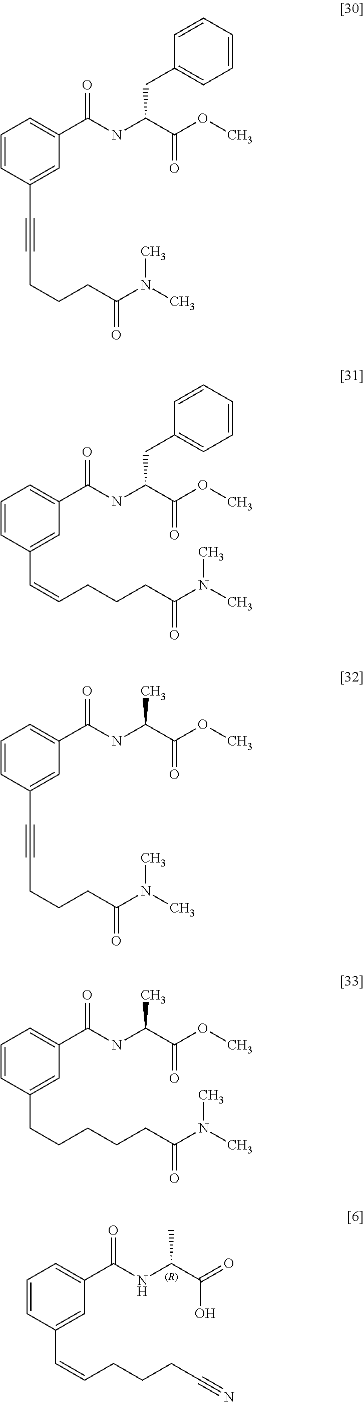 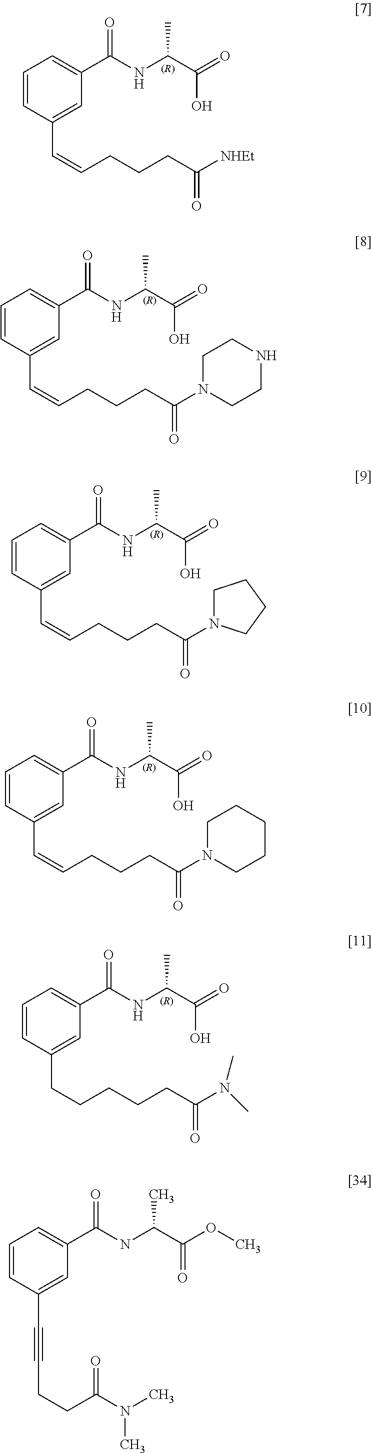 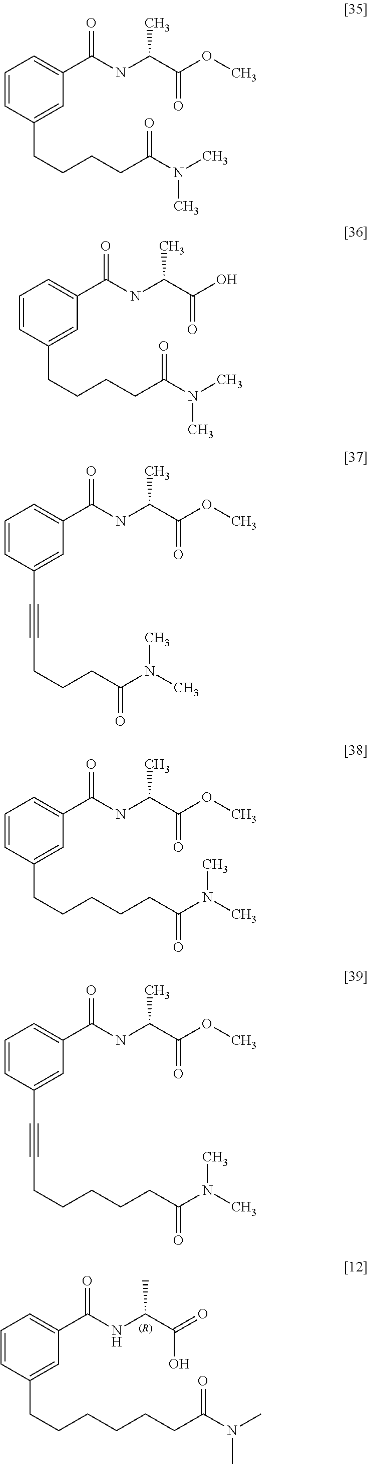 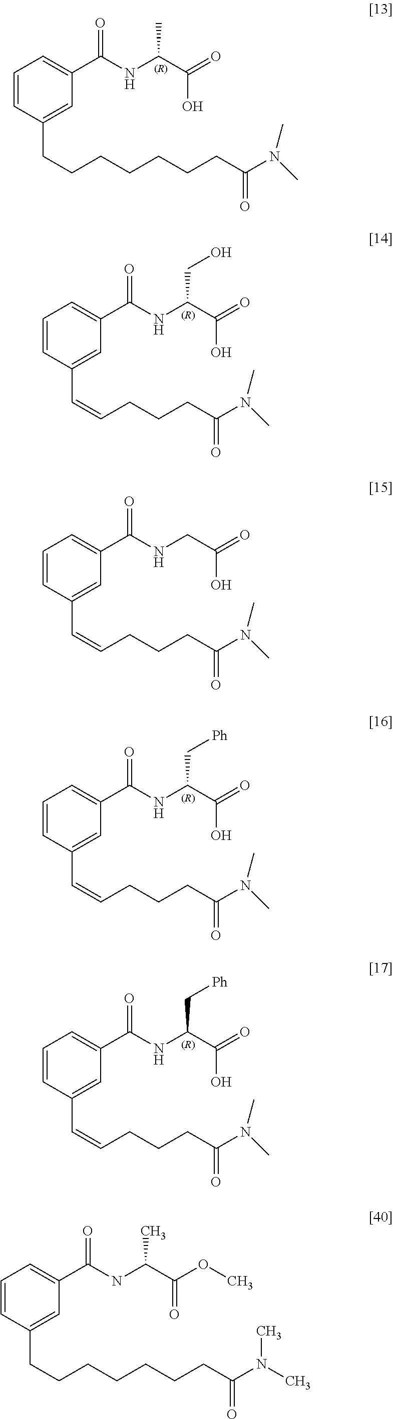 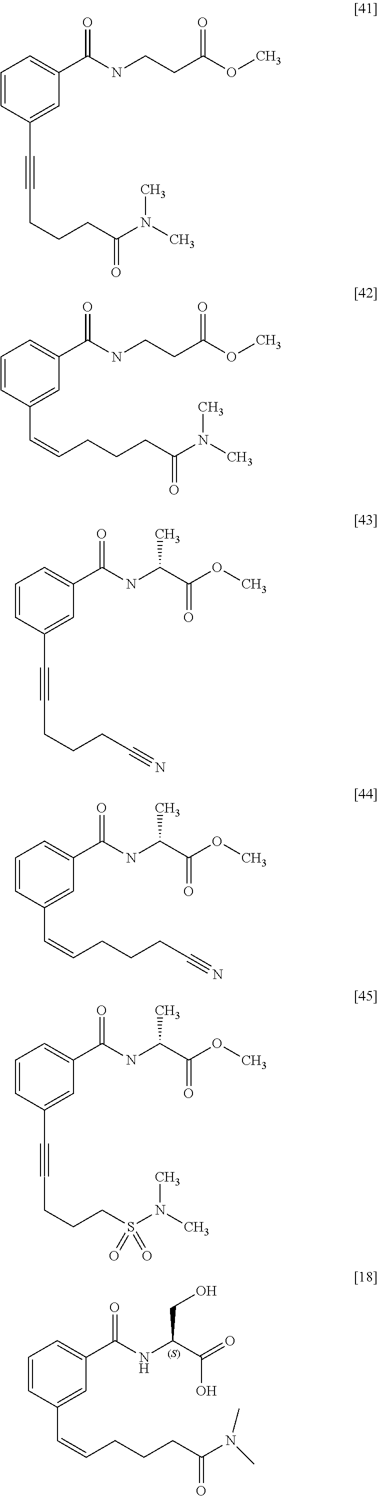 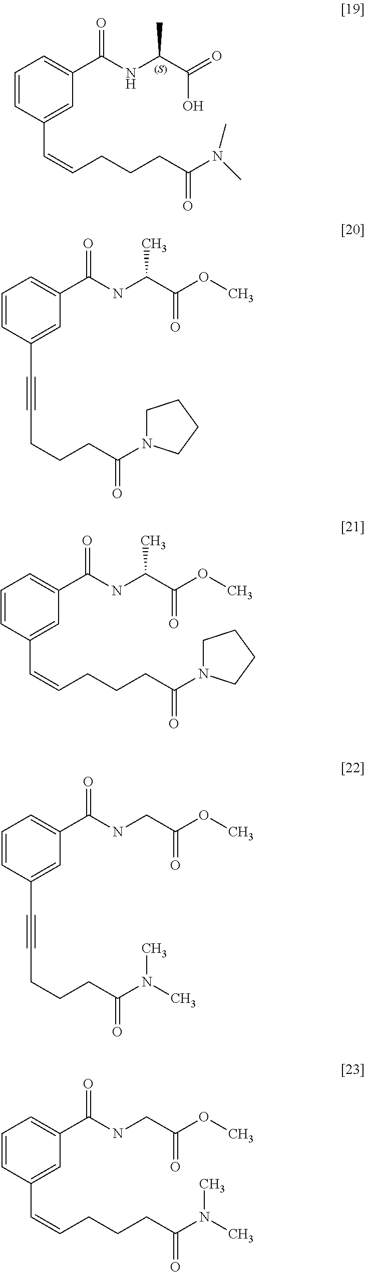 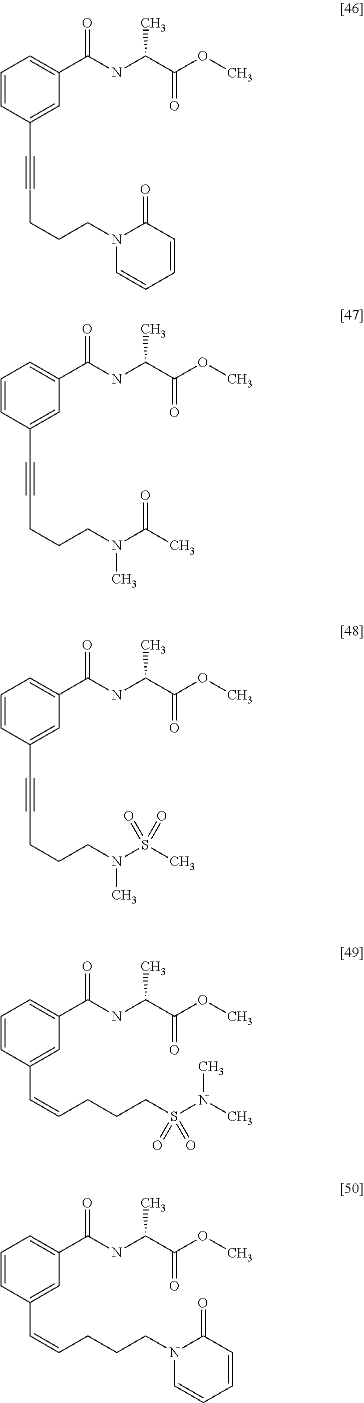 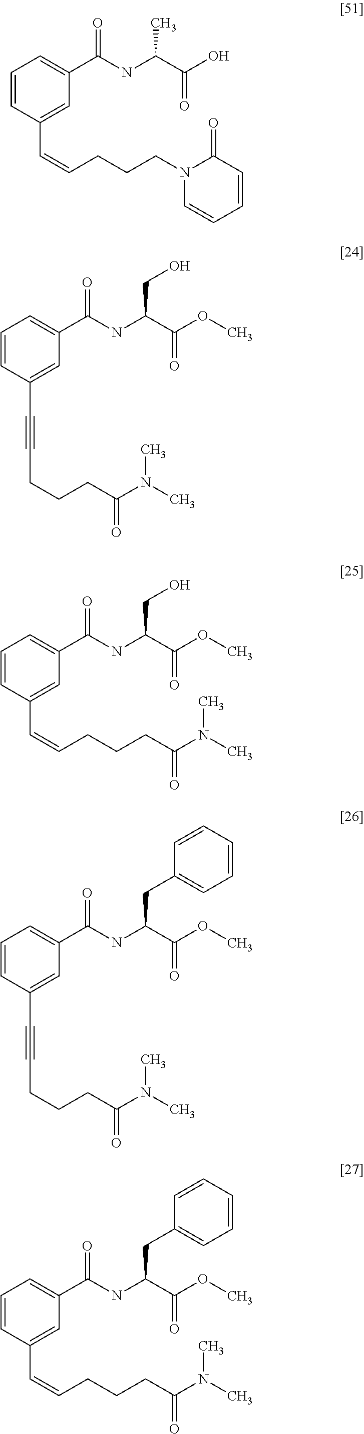 [SMILES: C[C@@H](NC(=O)c1cccc(/C=C\CCCn2ccccc2=O)c1)C(=O)O ; COC(=O)[C@H](CO)NC(=O)c1cccc(C#CCCCC(=O)N(C)C)c1 ; COC(=O)[C@H](CO)NC(=O)c1cccc(/C=C\CCCC(=O)N(C)C)c1 ; COC(=O)[C@H](Cc1ccccc1)NC(=O)c1cccc(C#CCCCC(=O)N(C)C)c1 ; COC(=O)[C@H](Cc1ccccc1)NC(=O)c1cccc(/C=C\CCCC(=O)N(C)C)c1] 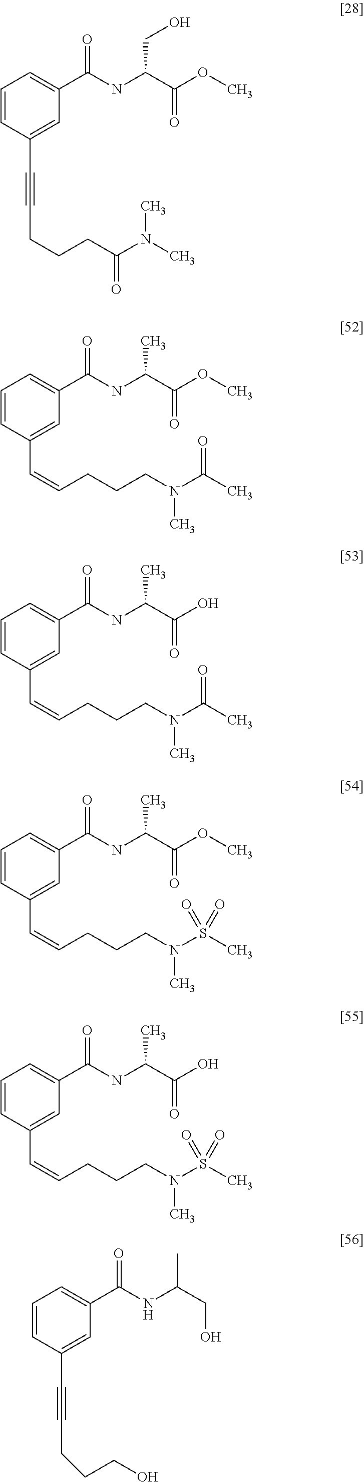 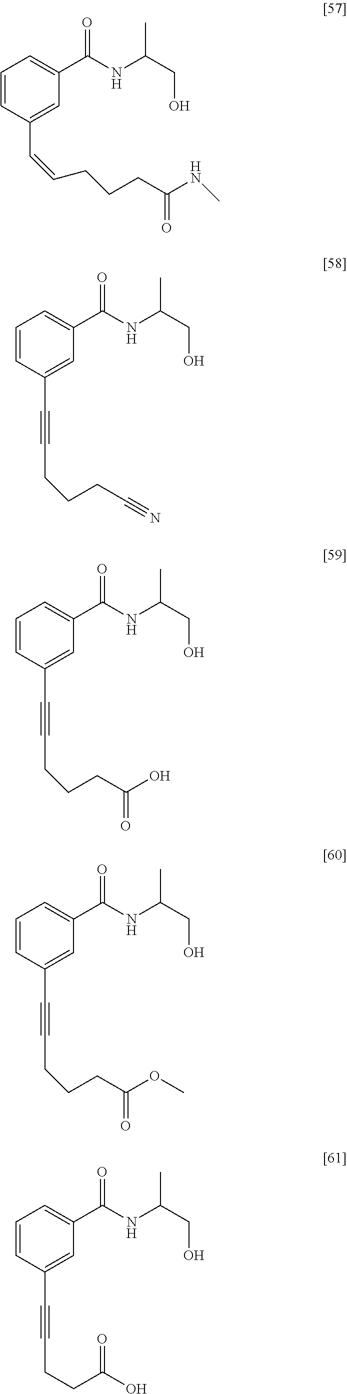 [SMILES: CNC(=O)CCC/C=C\c1cccc(C(=O)NC(C)CO)c1 ; CC(CO)NC(=O)c1cccc(C#CCCCC#N)c1 ; CC(CO)NC(=O)c1cccc(C#CCCCC(=O)O)c1 ; COC(=O)CCCC#Cc1cccc(C(=O)NC(C)CO)c1 ; CC(CO)NC(=O)c1cccc(C#CCCC(=O)O)c1] 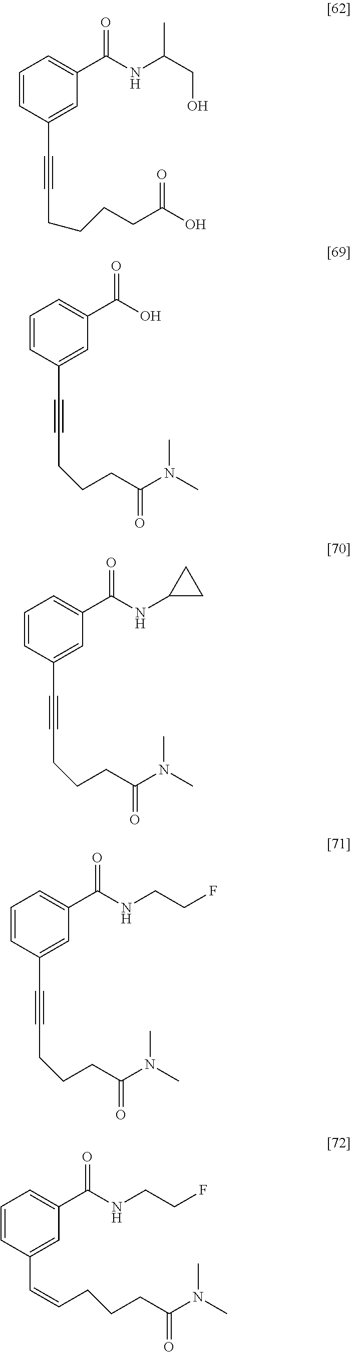 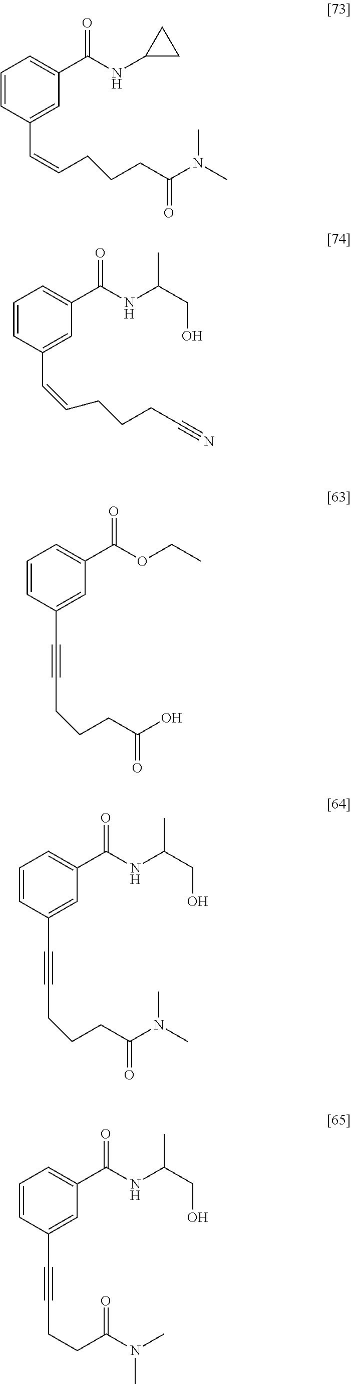 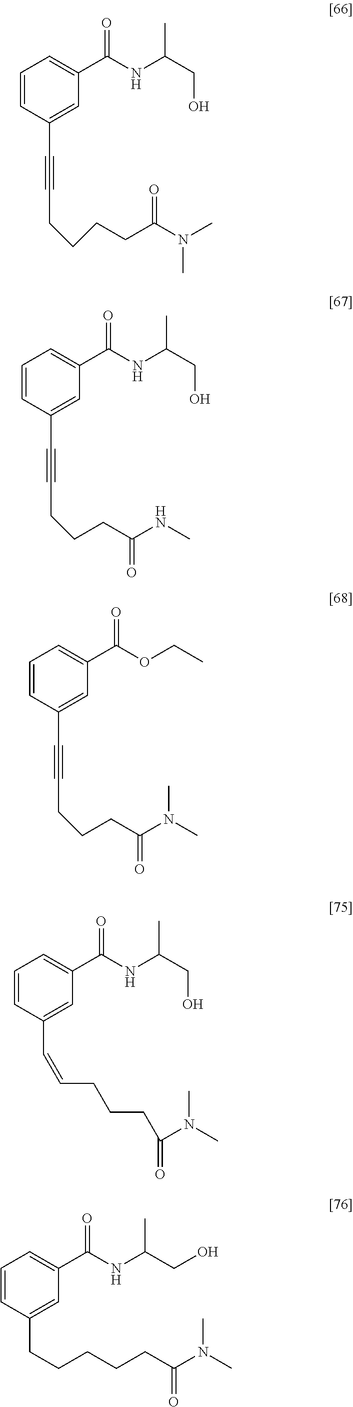 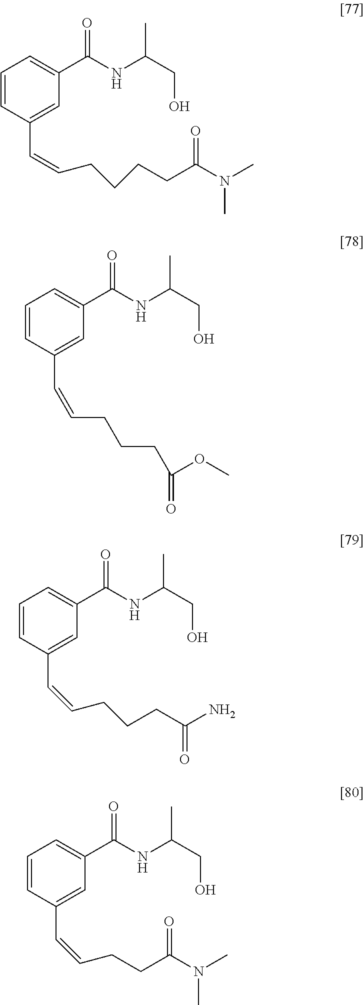
and pharmaceutically acceptable salts, solvates, prodrugs and enantiomers thereof.

19. The method according to claim 1 which is of the formulae [1], [75] or [57], or a pharmaceutically acceptable salt, solvate or prodrug thereof:

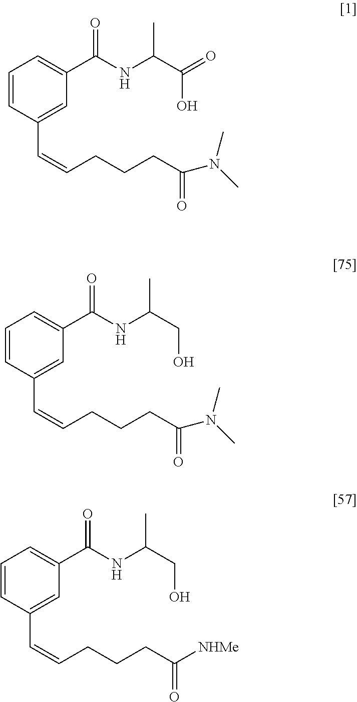 [SMILES: CC(NC(=O)c1cccc(/C=C\CCCC(=O)N(C)C)c1)C(=O)O ; CC(CO)NC(=O)c1cccc(/C=C\CCCC(=O)N(C)C)c1 ; CNC(=O)CCC/C=C\c1cccc(C(=O)NC(C)CO)c1]

20. The method according to claim 1 which is of the formulae [1a], [1b], [75a], [75b], [57a] or [57b], or a pharmaceutically acceptable salt, solvate or prodrug thereof:

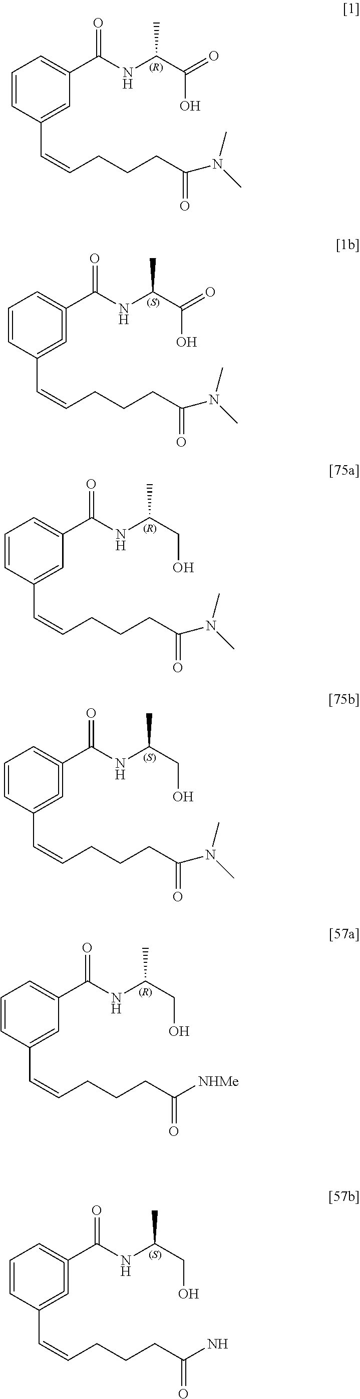

21. The method according to claim 1 wherein the compound is admixed with a pharmaceutically acceptable diluent, excipient or carrier.

22. The method according to claim 1 wherein the compound is a BK channel opener.

23. The method according to claim 1 wherein the disorder is glaucoma.

24. The method according to claim 1 wherein the disorder is tinnitus.

25. The method according to claim 1 wherein the disorder is Fragile X.

26. The method according to claim 1 wherein the disorder is diabetes.

27. The method according to claim 1 wherein the disorder is diabetic retinopathy.

28. The method according to claim 1 wherein the disorder is stroke.

29. The method according to claim 1 wherein the disorder is Age Related Macular Degeneration (AMD).

30. The method according to claim 1 wherein the disorder is retinitis pigmentosa.

31. The method according to claim 1 wherein the disorder is a psychosis.

32. The method according to claim 1 for use in treating vascular dysfunction.

33. The method according to claim 1 wherein the disorder is chronic obstructive pulmonary disorder (COPD).

34. The method according to claim 1 for providing neuroprotection.

35-36. (canceled)
